(19) United States
(12) Patent Application Publication
(10) Pub. No.: US 2006/0287381 A1
(43) Pub. Date: Dec. 21, 2006

(54) Enhanced indolinone based protein kinase inhibitors

(75) Inventors: Congxin Liang, Jupiter, FL (US); Yangbo Feng, Palm Beach Gardens, FL (US); Tomas Vojkovsky, Palm Beach Gardens, FL (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/441,537

(22) Filed: May 26, 2006

Related U.S. Application Data
(63) Continuation-in-part of application No. PCT/US04/39752, filed on Nov. 26, 2004.
(60) Provisional application No. 60/754,360, filed on Dec. 28, 2005.
(60) Provisional application No. 60/685,144, filed on May 26, 2005.

(51) Int. Cl. A61K 31/404; C07D 403/02

(52) U.S. Cl. 514/414; 548/465

(57) ABSTRACT

Hydroxy carboxy pyrrolyl-indolinone derivatives have enhanced and unexpected drug properties as inhibitors of protein kinases and are useful in treating disorders related to abnormal protein kinase activities such as cancer. More particularly, alpha-hydroxy-omega-(2-oxo-indolylidenemethyl-pyrrole-3′-carbonyl) amino alkanoic acid and amide derivatives have enhanced and unexpected drug properties as inhibitors of protein kinases with respect to their corresponding beta-hydroxy-omega-(2-oxo-indolylidenemethyl-pyrrole-3′-carbonyl) amino alkanoic acid and amide derivatives and are useful in treating disorders related to abnormal protein kinase activities such as cancer.

### FIELD OF INVENTION

The invention relates to protein kinase inhibitors and to their use in treating disorders related to abnormal protein kinase activities such as cancer and inflammation. More particularly, the invention relates to alpha-hydroxy-ω-(2-oxo-indolylidenemethyl-pyrrole-3′-carbonyl) amino alkanoic acid and amide derivatives and their pharmaceutically acceptable salts employable as protein kinase inhibitors.

### BACKGROUND

Protein kinases are enzymes that catalyze the phosphorylation of hydroxyl groups of tyrosine, serine, and threonine residues of proteins. Many aspects of cell life (for example, cell growth, differentiation, proliferation, cell cycle and survival) depend on protein kinase activities. Furthermore, abnormal protein kinase activity has been related to a host of disorders such as cancer and inflammation. Therefore, considerable effort has been directed to identifying ways to modulate protein kinase activities. In particular, many attempts have been made to identify small molecules that act as protein kinase inhibitors.

Several pyrrolyl-indolinone derivatives have demonstrated excellent activity as inhibitors of protein kinases (Larid et al. FASEB J. 16, 681, 2002; Smolich et al. Blood, 97, 1413, 2001; Mendel et al. Clinical Cancer Res. 9, 327, 2003; Sun et al. J. Med. Chem. 46, 1116, 2003). The clinical utility of these compounds has been promising, but has been partially compromised due to the relatively poor aqueous solubility and/or other drug properties. What is needed is a class of modified pyrrolyl-indolinone derivatives having both inhibitory activity and enhanced drug properties.

### SUMMARY

The invention is directed to hydroxy carboxy pyrrolyl-indolinone derivatives and to their use as inhibitors of protein kinases. It is disclosed herein that hydroxy carboxy pyrrolyl-indolinone derivatives have enhanced and unexpected drug properties that advantageously distinguish this class of compounds over known pyrrolyl-indolinone derivatives having protein kinase inhibition activity. It is also disclosed herein that hydroxy carboxy pyrrolyl-indolinone derivatives are useful in treating disorders related to abnormal protein kinase activities such as cancer.

One aspect of the invention is directed to a compound represented by Formula (I): 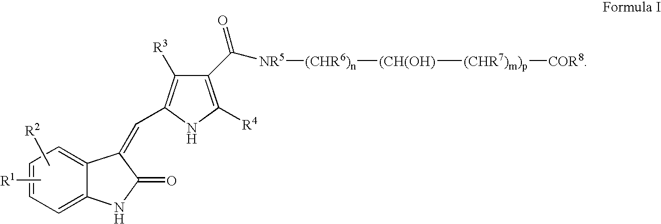
In Formula I, R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl; R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino; R³ is selected from the group consisting of hydrogen, (C1-C6) alkyl, (C6-C10) aryl, (C5-C10) heteroaryl, and amide; R⁴, R⁵ and R⁶ are independently selected from the group consisting of hydrogen and (C1-C6) alkyl; each R⁷ is independently selected from the group consisting of hydrogen, (C1-C6) alkyl and hydroxyl; R⁸ is selected from the group consisting of hydroxy, (C1-C6) O-alkyl, (C3-C8) O-cycloalkyl, and NR⁹R¹⁰; where R⁹ and R¹⁰ are independently selected from the group consisting of hydrogen, (C1-C6) alkyl, (C1-C6) hydroalkyl, (C1-C6) dihydroxyalkyl, (C1-C6) alkoxy, (C1-C6) alkyl carboxylic acid, (C1-C6) alkyl phosphoric acid, (C1-C6) alkyl sulfuric acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁹ and R¹⁰ together with N forms a (C5-C8) heterocyclic ring either unsubstituted or substituted with one or more hydroxyls, ketones, ethers, and carboxylic acids; and n and m are independently 0, 1, 2, or 3; p is 1, 2, or 3. Alternatively, this aspect of the invention may also be directed to a pharmaceutically acceptable salt, its tautomer, a pharmaceutically acceptable salt of its tautomer, or a prodrug of compounds represented by Formula (I). Key features of this aspect of the invention include the hydroxyl moiety or moieties between R⁶ and R⁷ and the carboxy moiety between R⁷ and R⁸. It is disclosed herein that these key features enhance the drug properties of the attached pyrrolyl-indolinone pharmacophore. Preferred species of this aspect of the invention include compounds represented by the following structures: 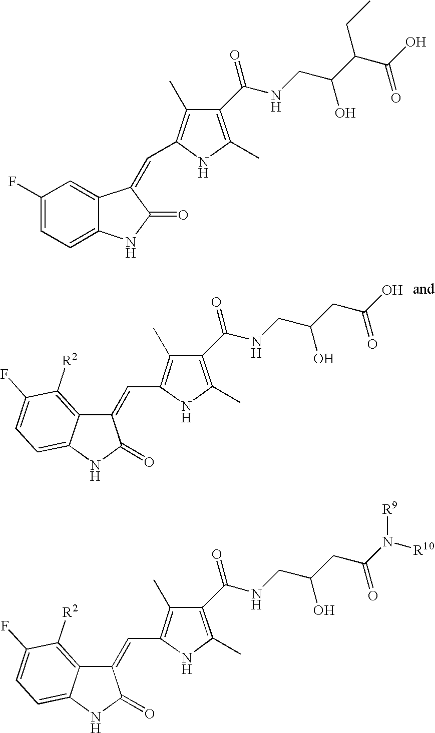
In the above structures, R² is selected from the group consisting of hydrogen and fluoro.

As illustrated above, this first aspect of the invention may be divided into two categories. The first category includes acids and esters; the second category includes amides.

One preferred embodiment of this first category may be represented by Formula (II): 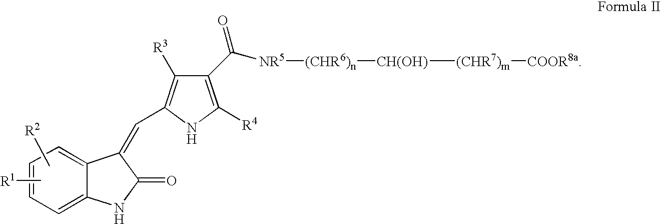
In Formula II, R^{8a} is selected from the group consisting of hydrogen, (C1-C6) alkyl, and (C3-C8) cycloalkyl. Within preferred species of this embodiment, R¹ and R² are independently selected from the group consisting of hydrogen and fluoro; R³ and R⁴ are methyl; R⁵, R⁶, R⁷ and R^{8a} are hydrogen; and n and m are independently 0, 1, or 2. Preferred species include compounds represented by the following structures:

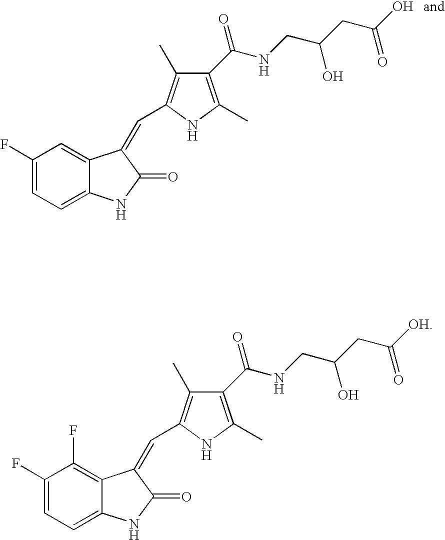

Another preferred embodiment of this first category may be represented by Formula (III): 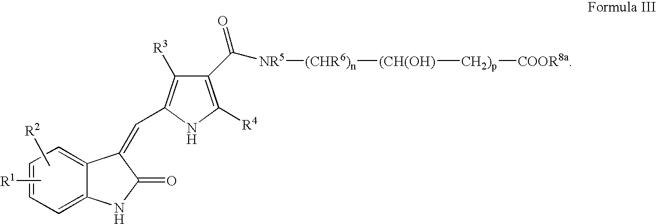
In Formula III, R^{8a} is selected from the group consisting of hydrogen, (C1-C6) alkyl, and (C3-C8) cycloalkyl. Within preferred species of this embodiment, R¹ and R² are independently selected from the group consisting of hydrogen and fluoro; R³ and R⁴ are methyl; R⁵, R⁶, and R^{8a} are hydrogen; and n and p are independently 1, or 2. Preferred species of this embodiment include compounds represented by the following structures: 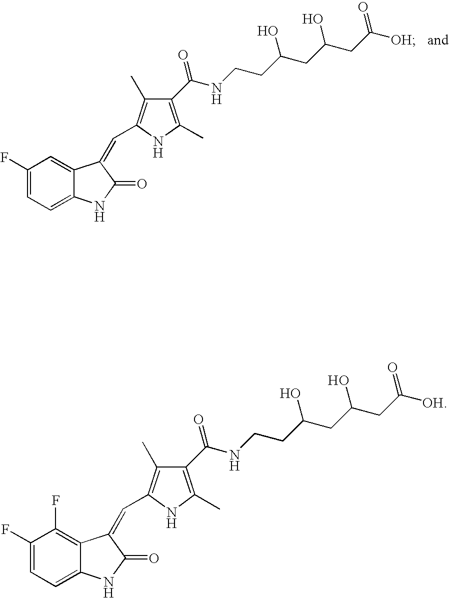
Preferred enantiomeric species of this embodiment of the invention include compounds represented by the following structures:

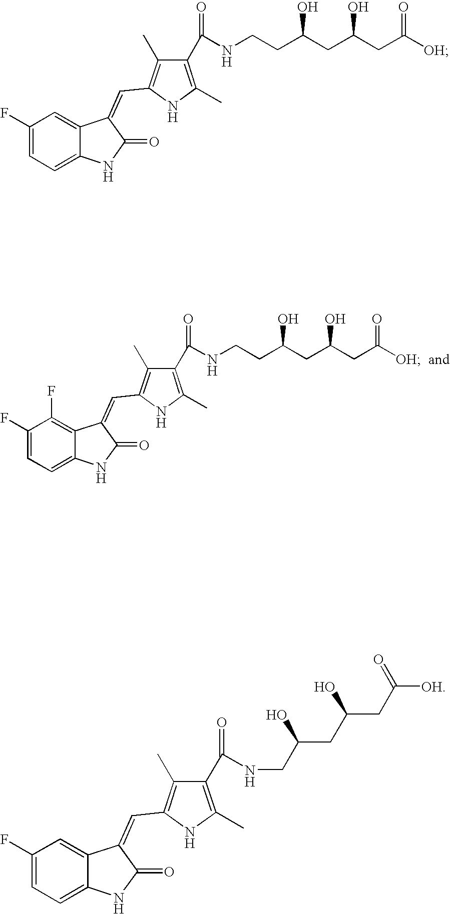

Another preferred embodiment of this first category may be represented by Formula (IIIa): 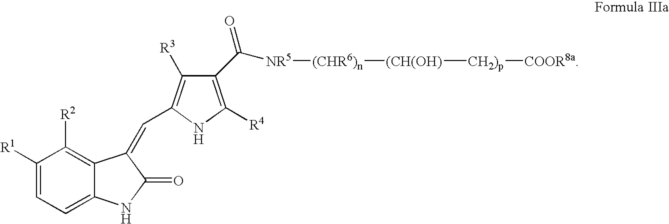
In Formula IIIa, R¹ and R² are independently selected from the group consisting of hydrogen and fluoro; R³ and R⁴ are methyl; R⁵, R⁶, and R^{8a} are hydrogen; and n and p are 2. Within this embodiment, each species may exist either as the acid or as the cyclic lactone and they may co-exist in solution or in vivo. Furthermore, in the above examples the stereochemistry at the carbon atom carrying a hydroxyl group is either RS, R, or S. The preferred stereochemistry is R.

An alternative of the above preferred embodiment of this first category may be represented by Formula (IIIb): 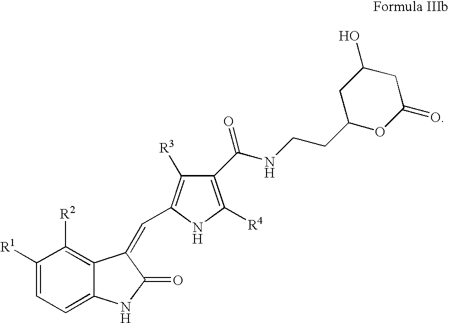
In Formula IIIb, R¹ and R² are independently selected from the group consisting of hydrogen and fluoro; and R³ and R⁴ are methyl. Preferred species of this embodiment include compounds represented by the following structures:

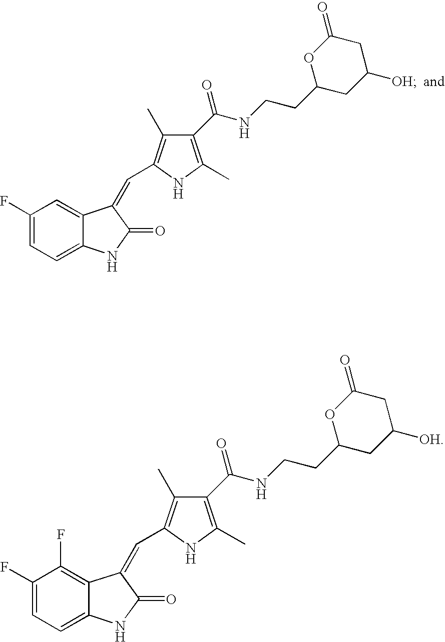

The second category of the first aspect of the invention is embodied by a compound, salt, tautomer, or prodrug according to claim 1 represented by Formula (IV): 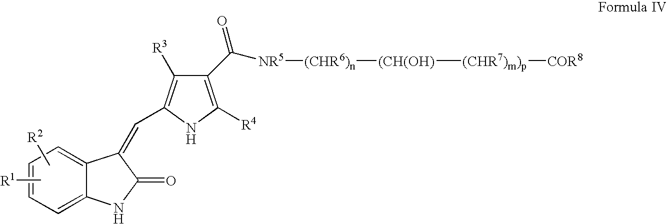
wherein R⁸ is NR⁹R¹⁰. In preferred embodiments of this aspect of the invention, R¹ and R² are independently selected from the group consisting of hydrogen, halo, cyano; R³, R⁴, R⁵ and R⁶ are independently hydrogen or (C1-C6))alkyl; R⁷ is hydrogen, or hydroxyl; n, and p are independently 1, or 2; m is 0 or 1; and R⁹ and R¹⁰ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphoric acid, (C1-C6) alkyl sulfuric acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁹ and R¹⁰ together with N forms a (C5-C8) heterocyclic ring either unsubstituted or substituted with one or more hydroxyls, ketones, ethers, and carboxylic acids. Preferred examples of R⁸ include the following radicals: 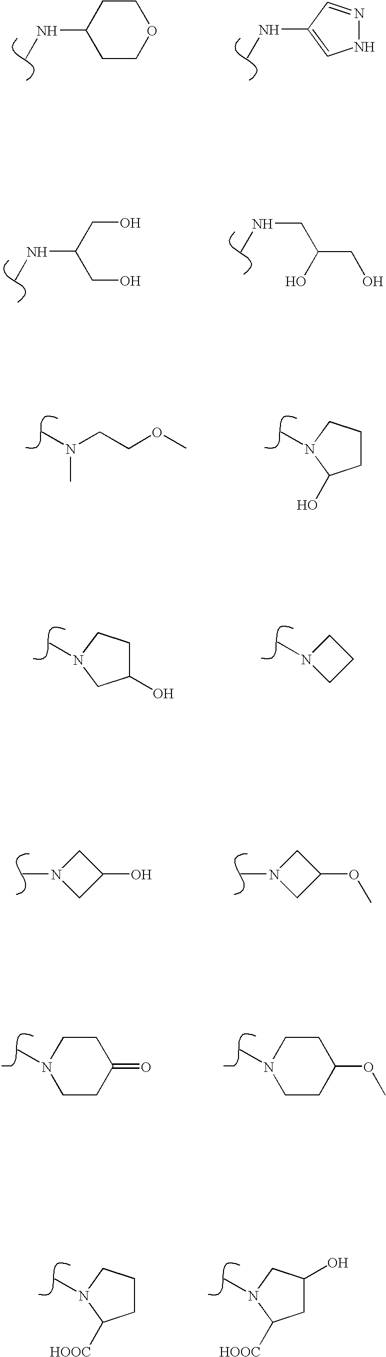 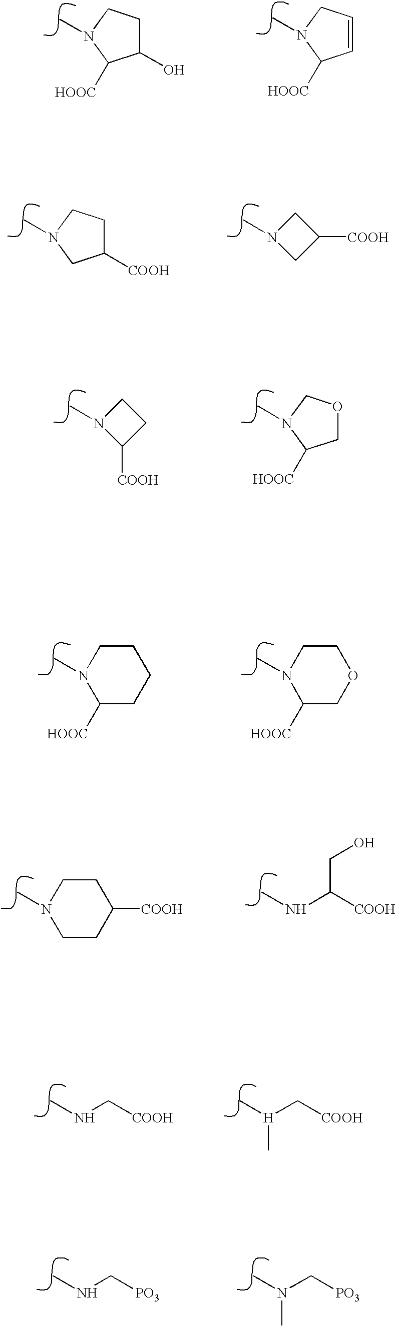 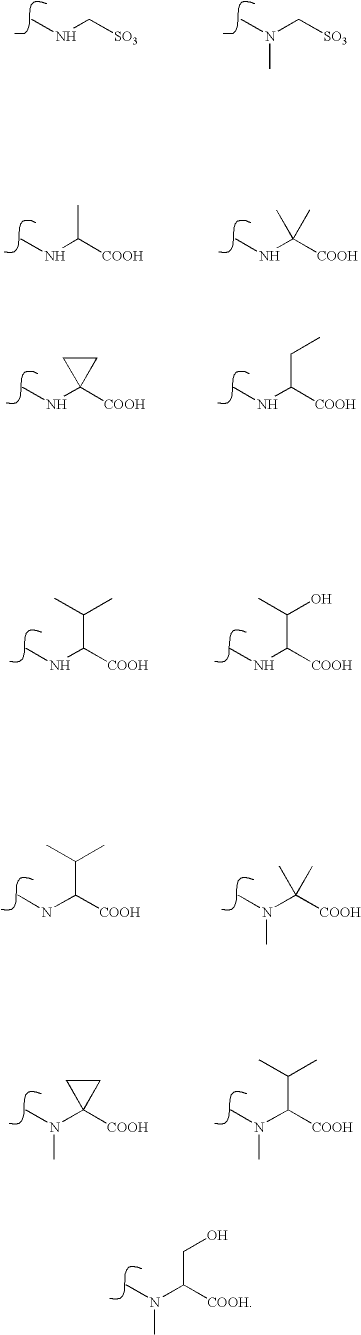
Preferred species of this embodiment may be selected from the group represented by the following structures: 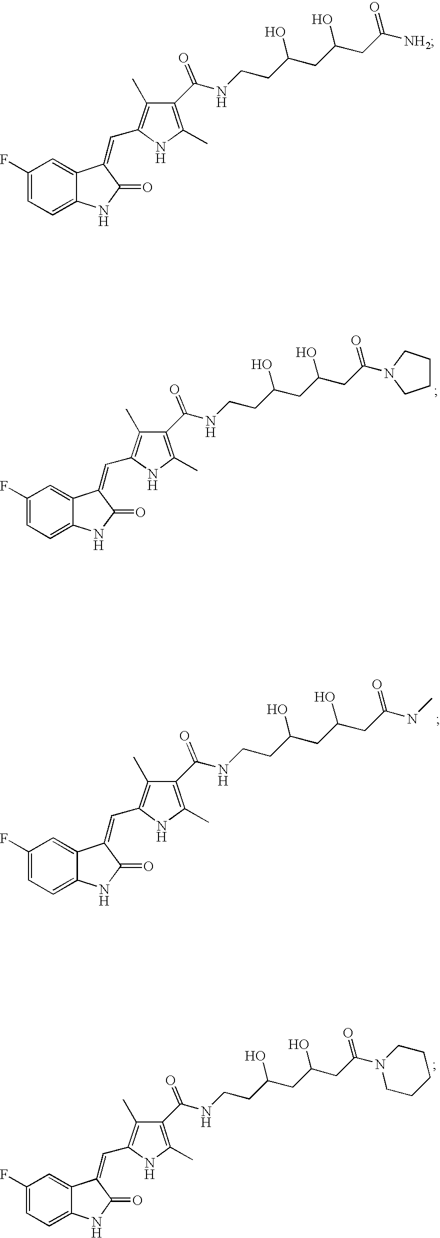 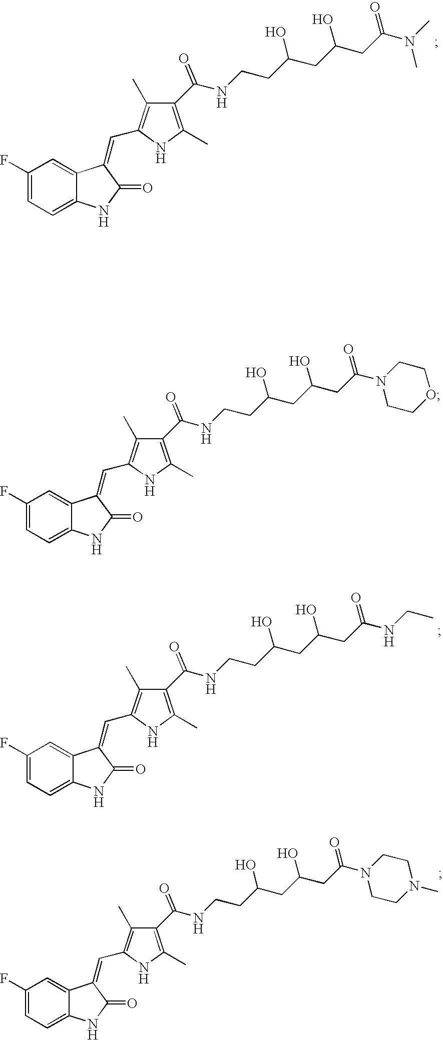 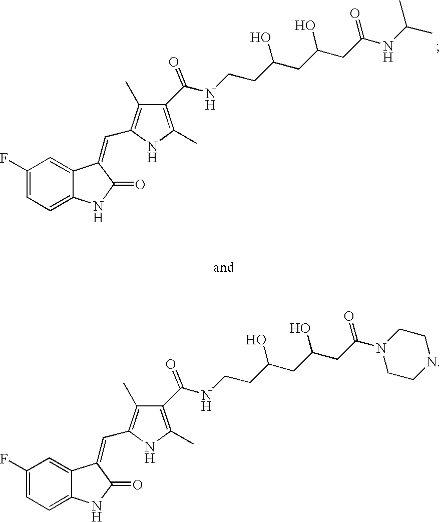
Further preferred species of this embodiment of the invention may be selected from the group represented by the following structures: 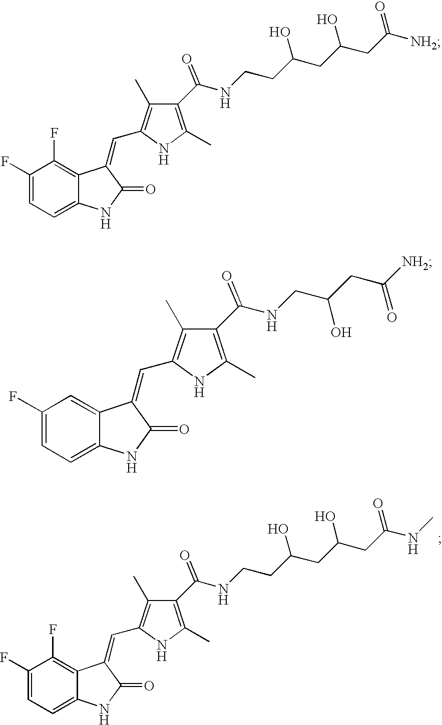 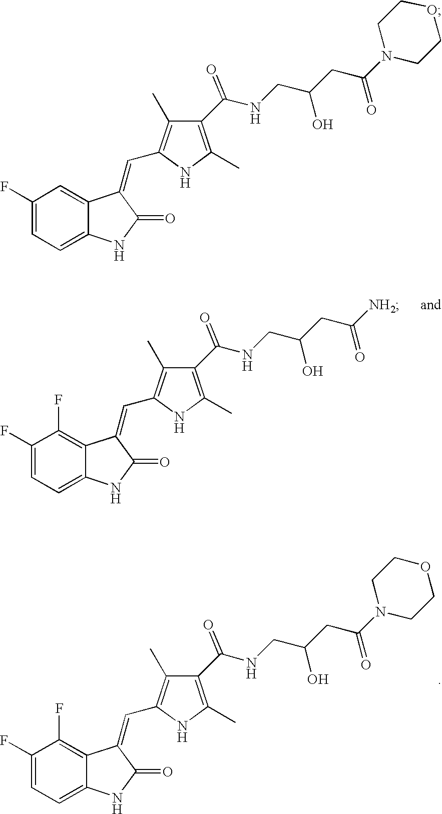
Further preferred species of this embodiment of the invention may be selected from the group represented by the following structures: 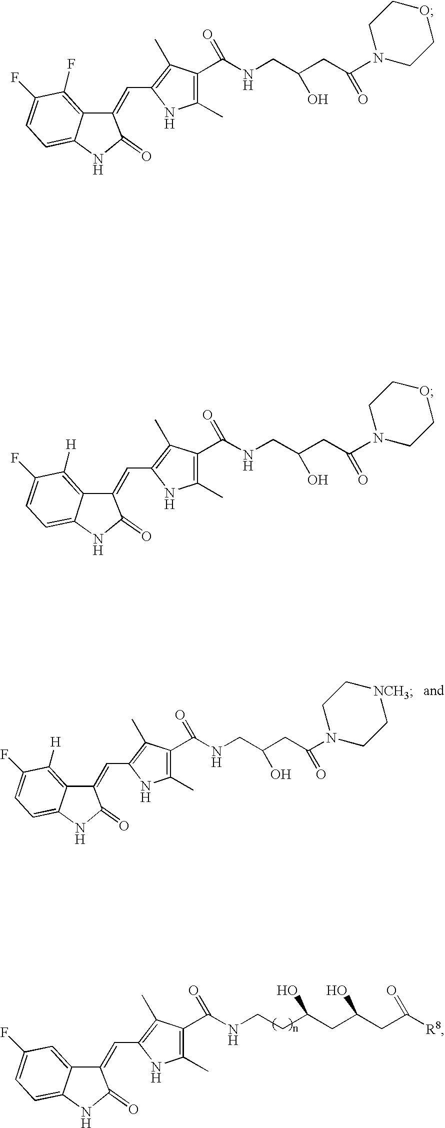
wherein n is 0, 1, or 2. Further preferred species of this embodiment of the invention may be selected from the group represented by the following structures: 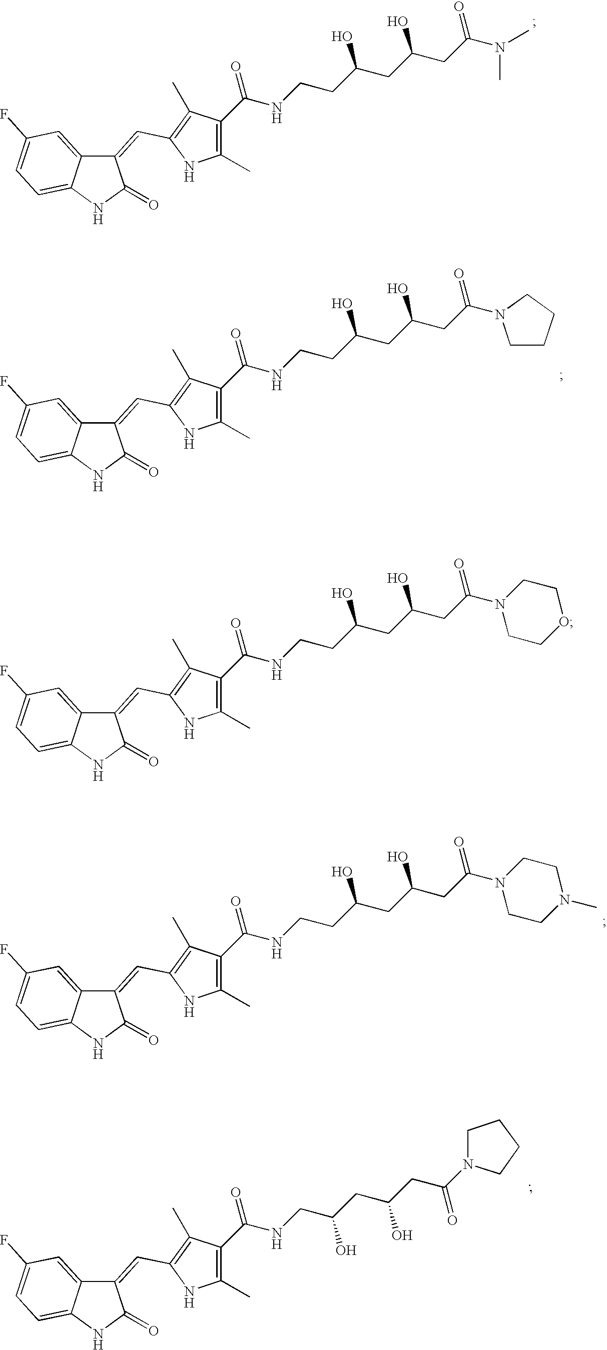 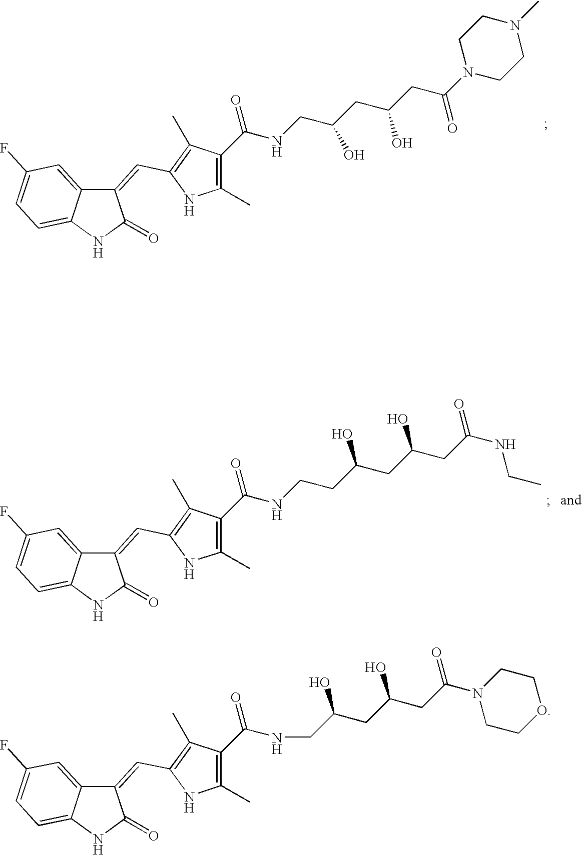
Further preferred species of this embodiment of the invention may be selected from the group represented by the following structures: 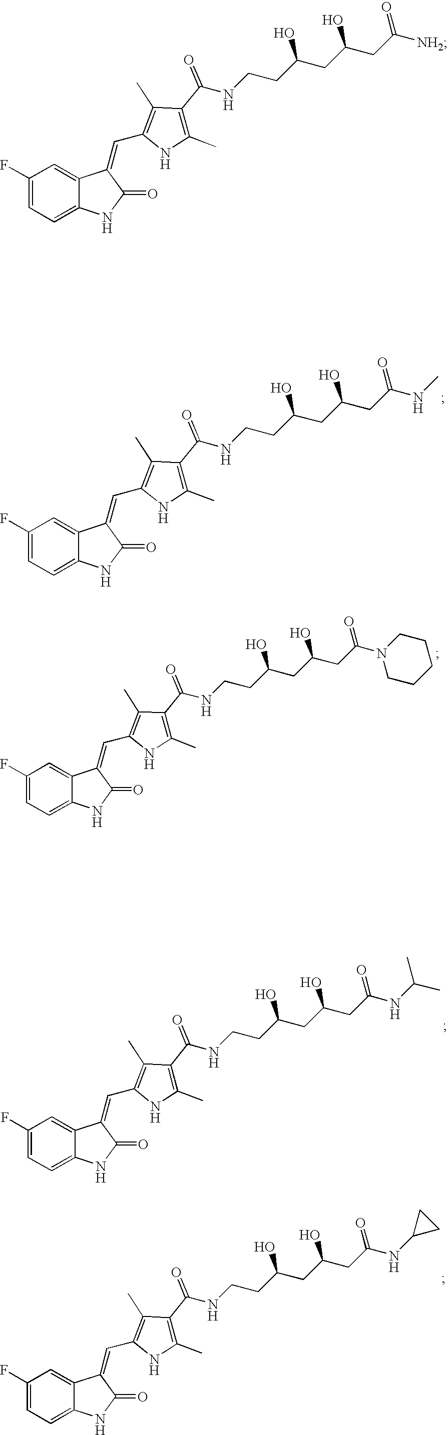 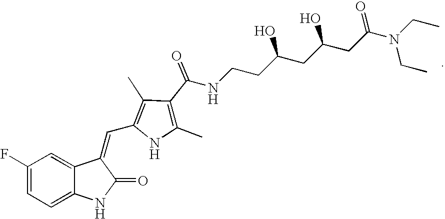
Further preferred species of this embodiment of the invention may be selected from the group represented by the following structures: 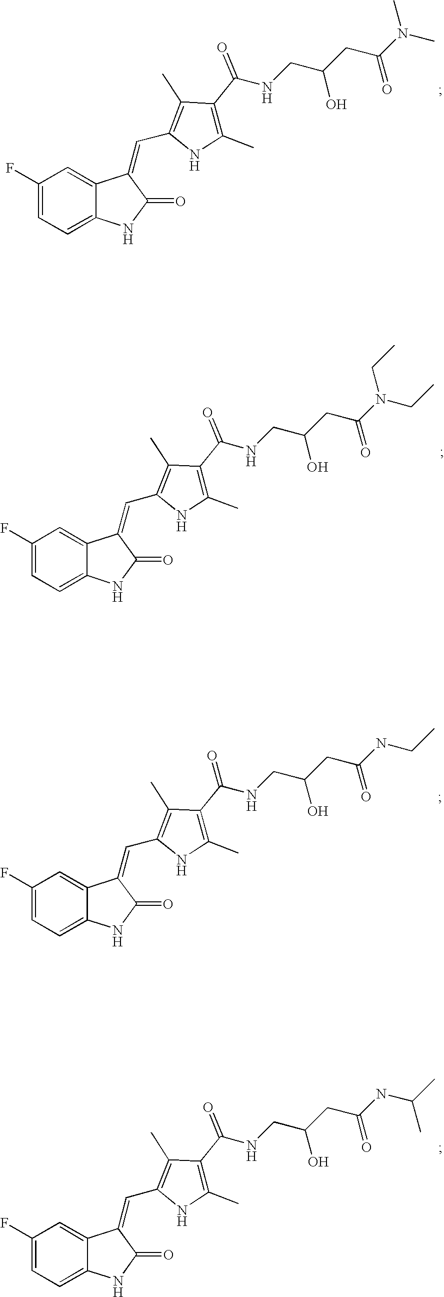 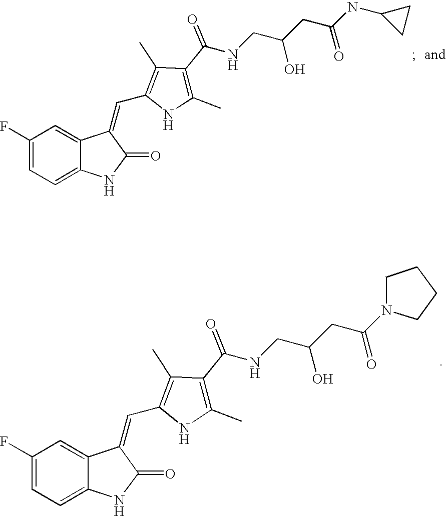
Further preferred species of this embodiment of the invention may be selected from the group represented by the following structures: 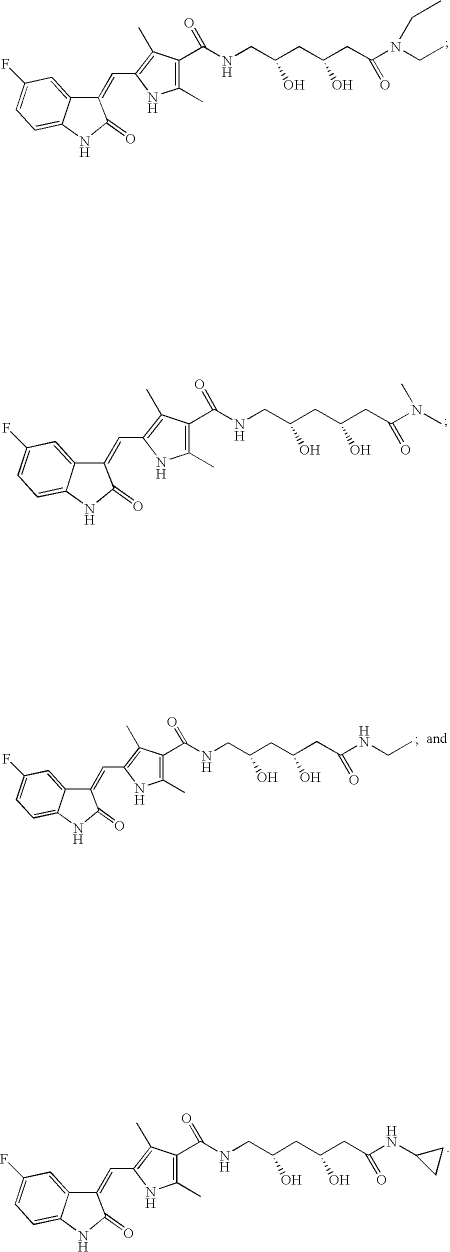
Further preferred species of this embodiment of the invention may be selected from the group represented by the following structures: 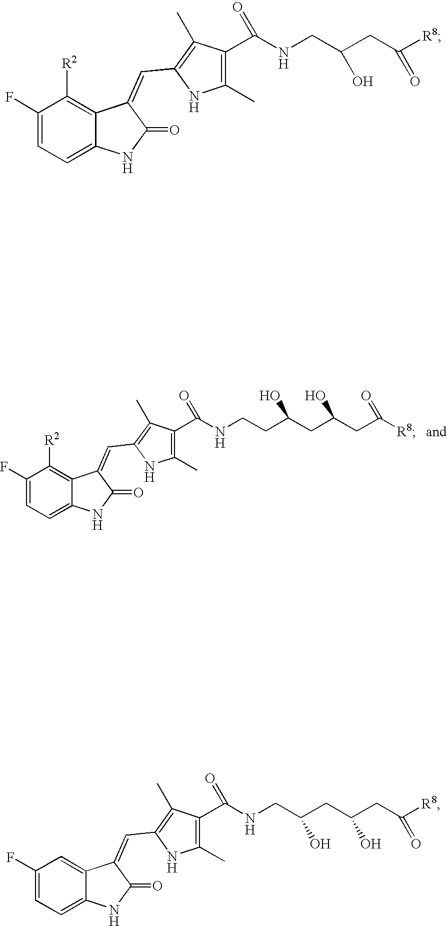
wherein R² is selected from the group consisting of hydrogen and fluoro; and R⁸ is selected from the group consisting of radicals represented by the following structures:

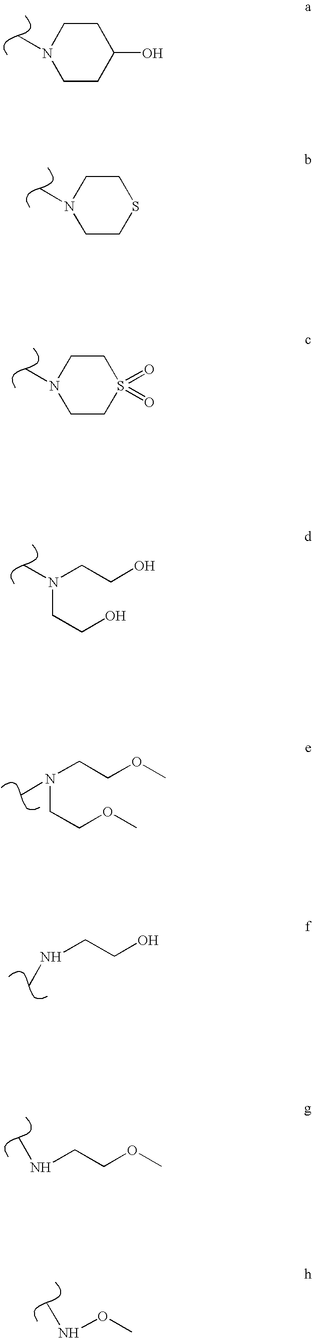

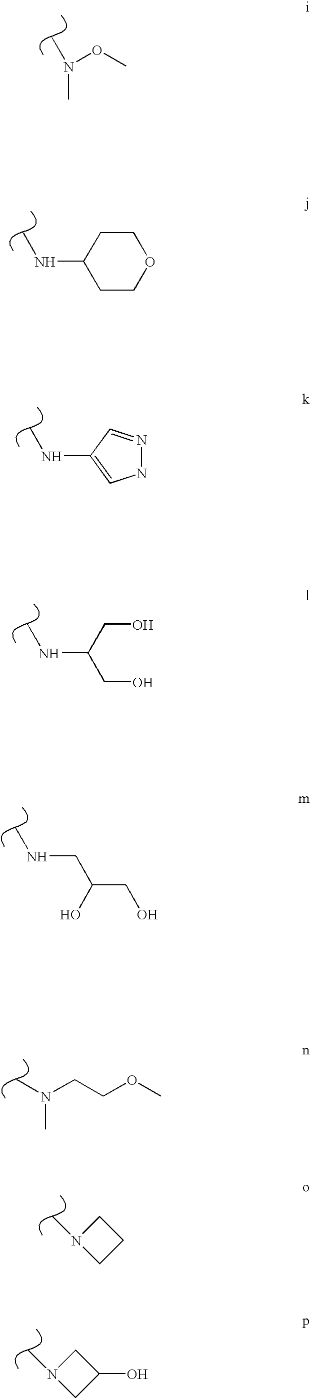

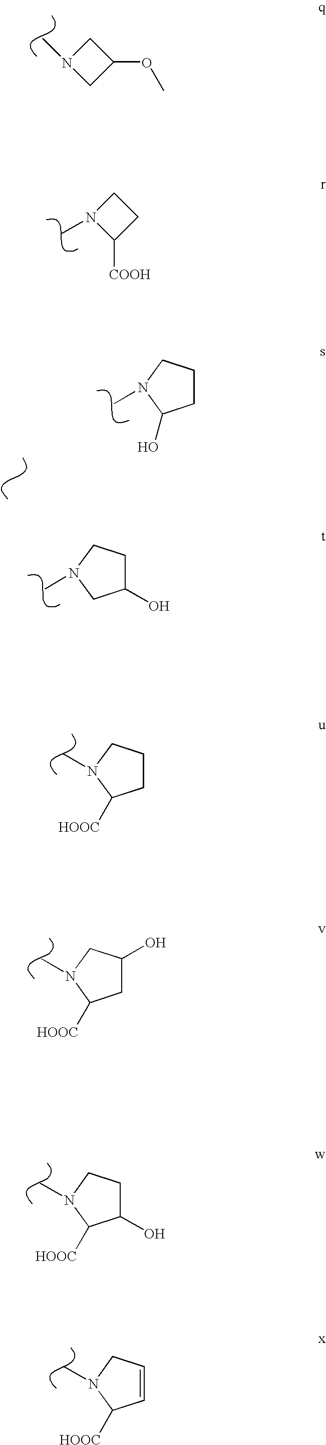

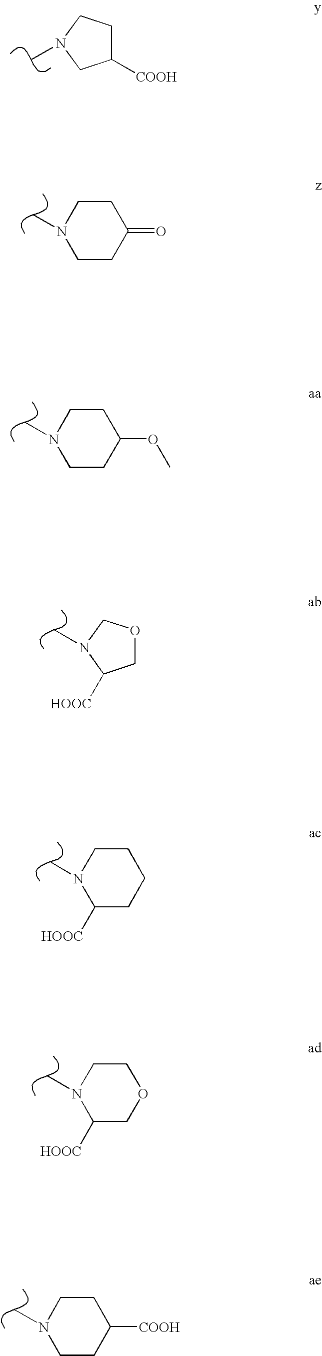

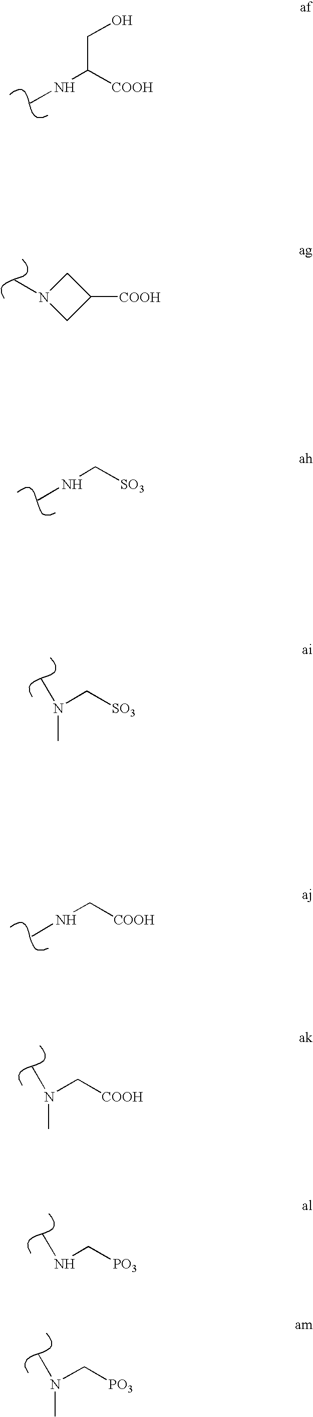

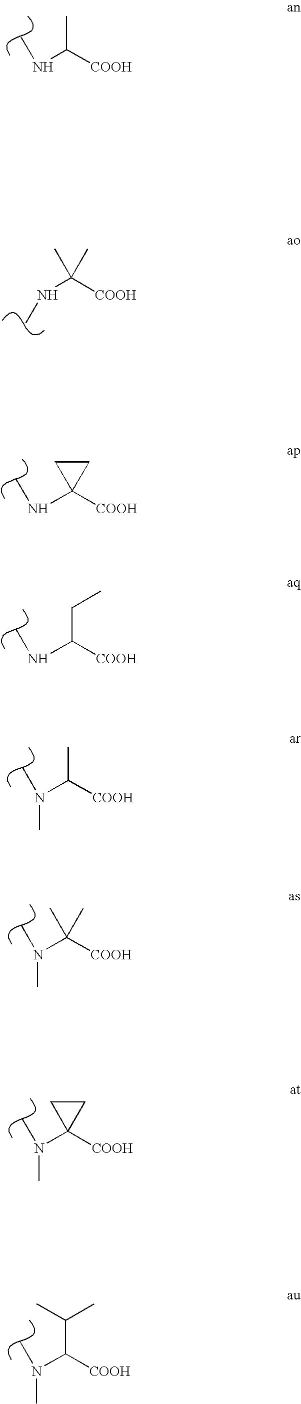

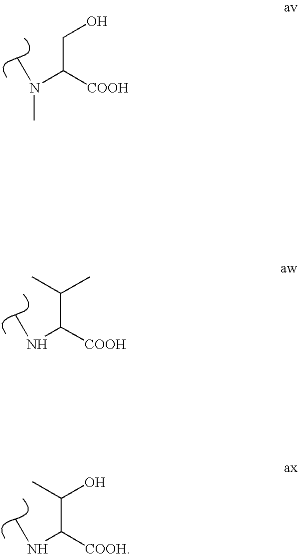

Another category within this first aspect of the invention is directed to alpha-hydroxy-omega-(2-oxo-indolylidenemethyl-pyrrole-3′-carbonyl) amino alkanoic acid and amide derivatives and to their use as inhibitors of protein kinases. It is disclosed herein that alpha-hydroxy-ω-(2-oxo-indolylidenemethyl-pyrrole-3′-carbonyl) amino alkanoic acid and amide derivatives have enhanced and unexpected drug properties that advantageously distinguish this class of compounds over known pyrrolyl-indolinone derivatives having protein kinase inhibition activity and over their corresponding beta hydroxy-ω-(2-oxo-indolylidenemethyl-pyrrole-3′-carbonyl) amino alkanoic acid and amide derivatives. It is also disclosed herein that alpha-hydroxy-ω(2-oxo-indolylidenemethyl-pyrrole-3′-carbonyl) amino alkanoic acid and amide derivatives are useful in treating disorders related to abnormal protein kinase activities such as cancer.

One such category within this first aspect of the invention is directed to a compound represented by Formula (V): 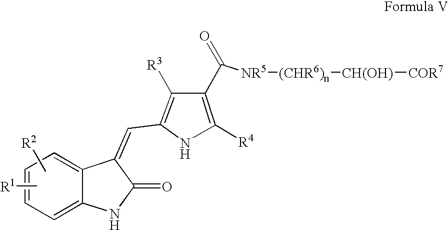
In Formula (V), R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl; R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino; R³ is selected from the group consisting of hydrogen, (C1-C6) alkyl, (C6-C10) aryl, (C5-C10) heteroaryl, and amide; R⁴, R⁵ and R⁶ are independently selected from the group consisting of hydrogen and (C1-C6) alkyl; R⁷ is selected from the group consisting of hydroxy, (C1-C6) O-alkyl, (C3-C8) O-cycloalkyl, and NR⁸R⁹; where R⁸ and R⁹ are independently selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids; and n is 1, 2, or 3. Alternatively, this aspect of the invention may be directed to a pharmaceutically acceptable salt, its tautomer, a pharmaceutically acceptable salt of its tautomer, or a prodrug of the compound of Formula (V). Preferred species of the invention include compounds represented by the following structures: 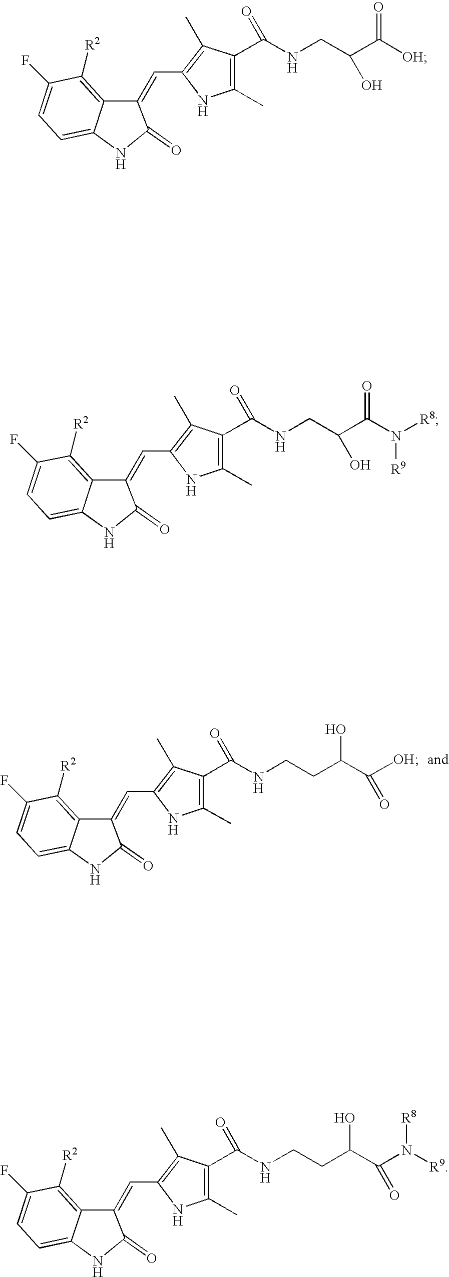
In the above structures, R² is selected from the group consisting of hydrogen and fluoro. More particularly, a preferred stereoisomer is represented by the following structure: 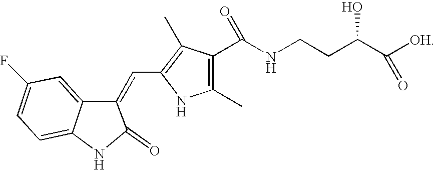
A first subgenus of this aspect of the invention is represented by Formula (VI): 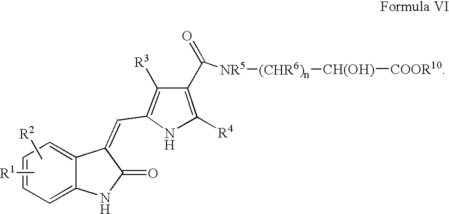
In Formula (VI), R¹⁰ is selected from the group consisting of hydrogen, (C1-C6) alkyl, and (C3-C8) cycloalkyl. In preferred species of this first subgenus, R¹ and R² are independently selected from the group consisting of hydrogen and fluoro;
R³ and R⁴ are methyl; R⁵, R⁶, and R¹⁰ are hydrogen; and n is 1 or 2. Preferred species are represented by the following compounds: 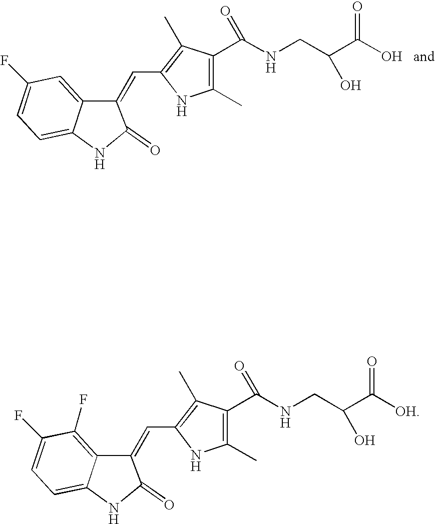
   A preferred chiral species is represented by the following compound: 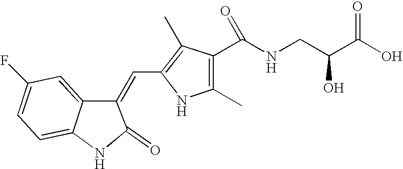
   A second subgenus of this aspect of the invention is directed to a compound according to Formula (VII) or a salt, tautomer, or prodrug thereof: 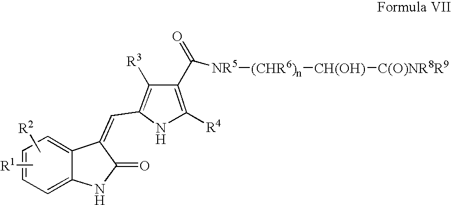
   In preferred species of this second subgenus, R¹ and R² are independently selected from the group consisting of hydrogen, halo, cyano; R³, R⁴, R⁵ and R⁶ are independently hydrogen or (C1-C6))alkyl; n is 1 or 2; and R⁸ and R⁹ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids. Preferred species of the second subgenus are represented by the following structures: 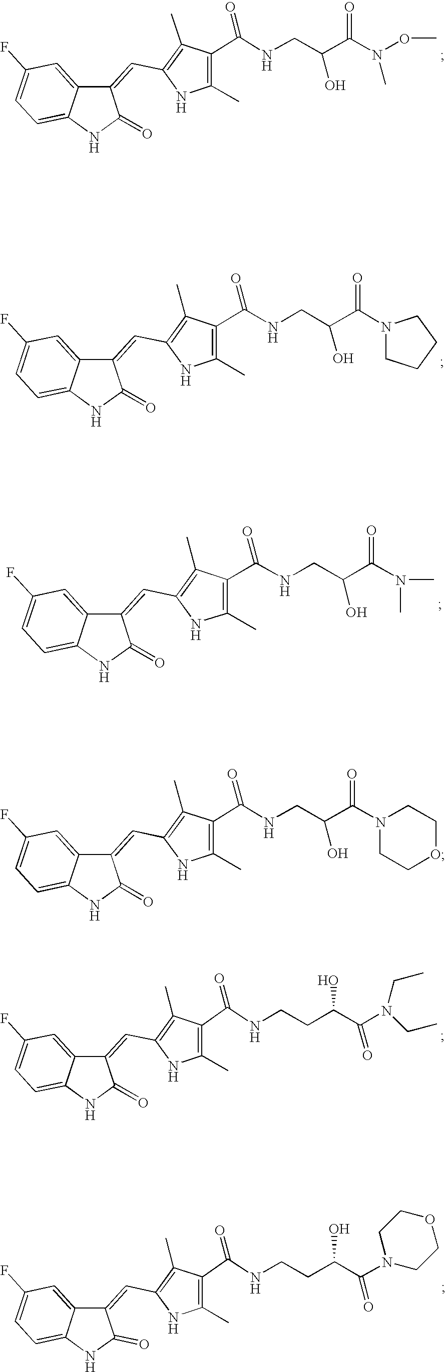 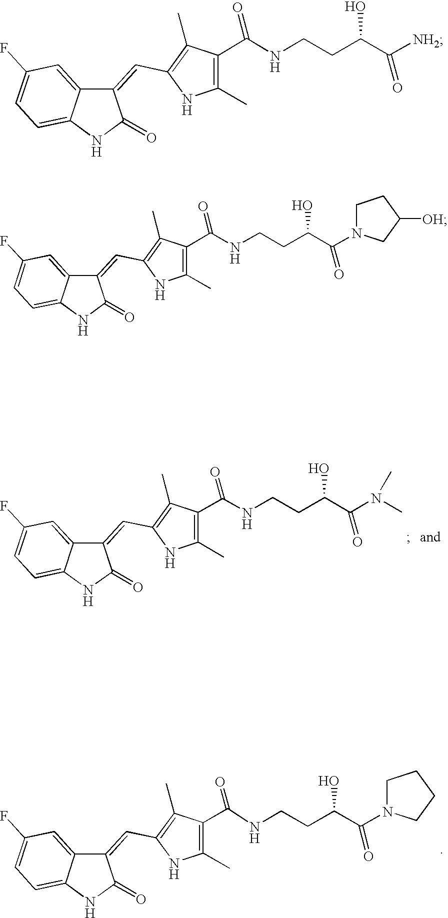
   In a first subset of the second subgenus, n is 1. Preferred species within this first subset are represented by the following structures: 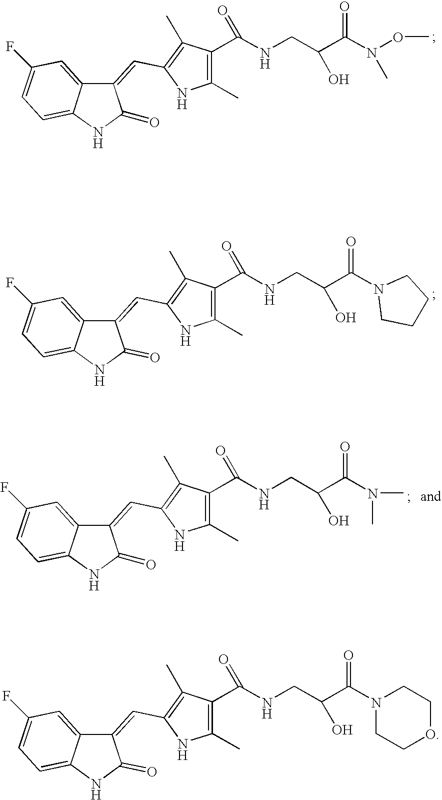
   Preferred chiral species within the first subset of the second subgenus are represented by the following structures: 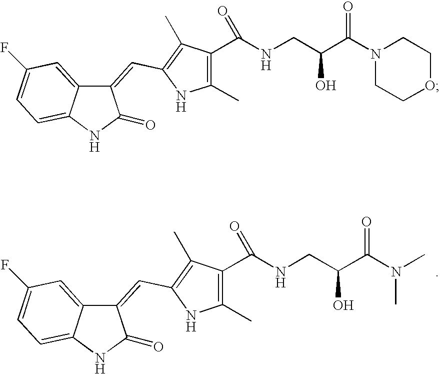
   Further preferred chiral species within the first subset of the second subgenus are represented by the following structures: 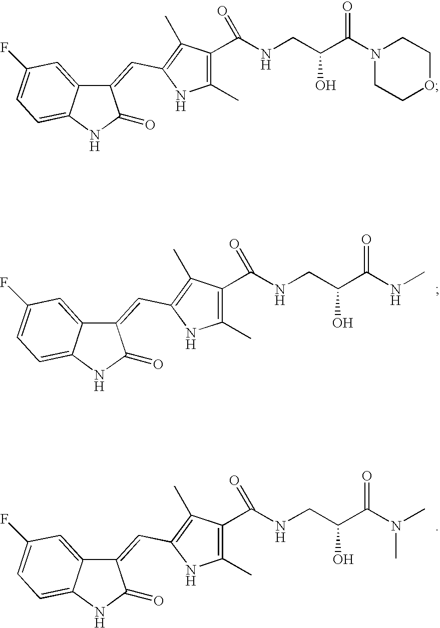
   In a second subset of the second subgenus, n is 2. Preferred species within this first subset are represented by the following structures: 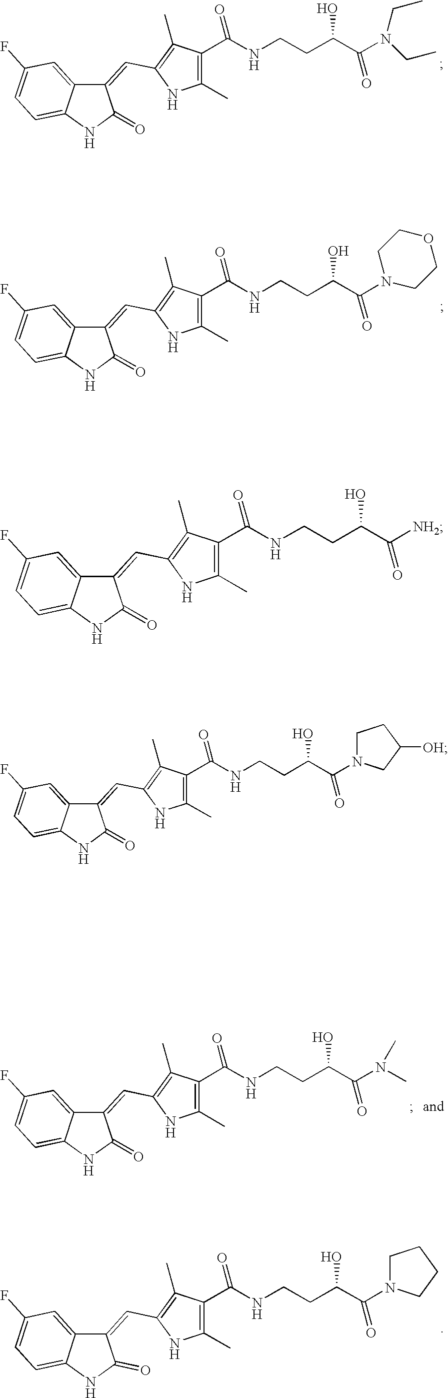
   Further preferred species of the first aspect of the invention are represented by the following structures: 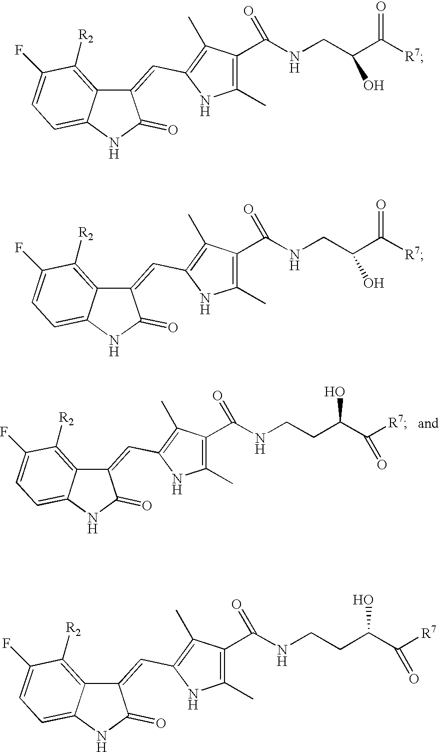
   In the above structures, R² is selected from the group consisting of hydrogen and fluoro; and R⁷ is selected from the group consisting of hydroxyl or radicals represented by the following structures:

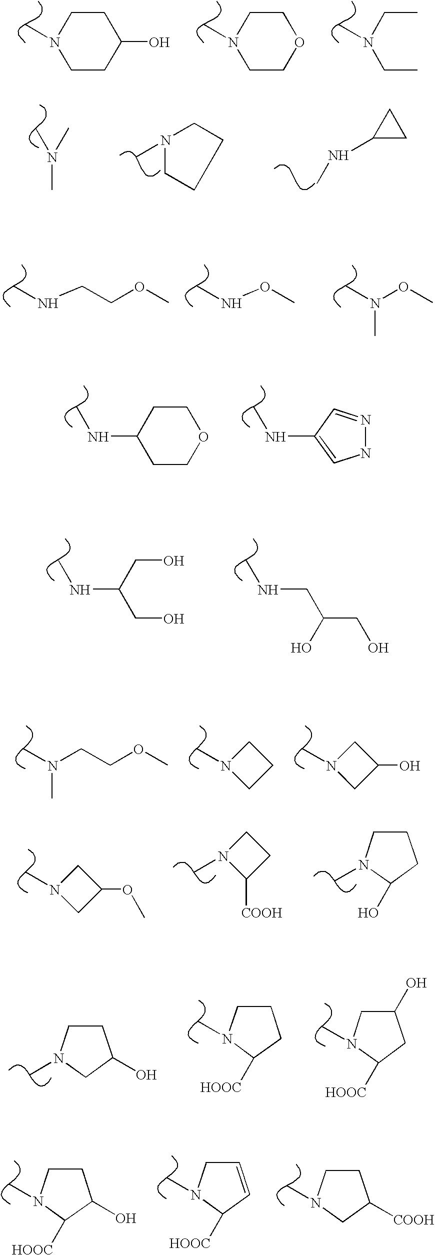

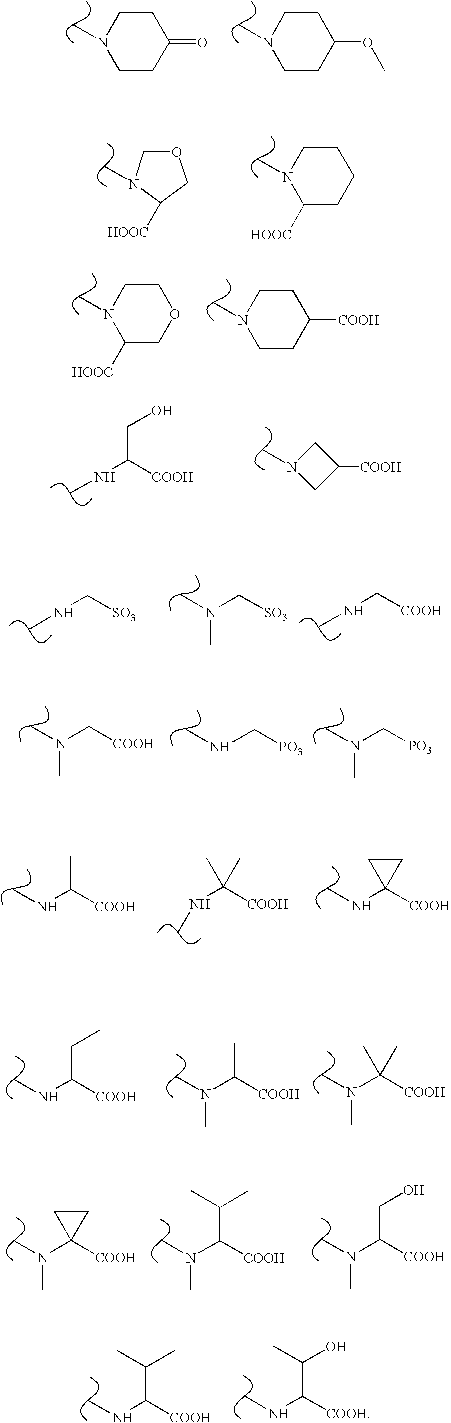

Provisos may apply to any of the above categories or embodiments wherein any one or more of the other above described embodiments or species may be excluded from such categories or embodiments.

A second aspect of the invention is directed to a method for the modulation of the catalytic activity of a protein kinase with a compound or salt represented by Formulas I-VII, above. In a preferred mode of the second aspect of the invention, said protein kinase is selected from the group of receptors consisting of VEGF, PDGF, c-kit, Flt-3, Axl, and TrkA.

Utility:

The present invention provides compounds capable of regulating and/or modulating protein kinase activities of, but not limited to, VEGFR and/or PDGFR. Thus, the present invention provides a therapeutic approach to the treatment of disorders related to the abnormal functioning of these kinases. Such disorders include, but not limited to, solid tumors such as glioblastoma, melanoma, and Kaposi's sarcoma, and ovarian, lung, prostate, pancreatic, colon and epidermoid carcinoma. In addition, VEGFR/PDGFR inhibitors may also be used in the treatment of restenosis and diabetic retinopathy.

Furthermore, this invention relates to the inhibition of vasculogenesis and angiogenesis by receptor-mediated pathways, including the pathways comprising VEGF receptors, and/or PDGF receptors. Thus the present invention provides therapeutic approaches to the treatment of cancer and other diseases which involve the uncontrolled formation of blood vessels.

### BRIEF DESCRIPTION OF DRAWINGS

FIG. 1 illustrates a scheme showing the synthesis of the acid 1-3 and the corresponding amides, 1-4. The starting carboxylic acid is synthesized according to the supplemental material of Sun, L.; et al., J. Med. Chem. 2003, 46, 1116-1119.

FIG. 2 illustrates a scheme showing the synthesis of the amide series, 2-3.

FIG. 3 shows example compounds and some of their activities against KDR.

FIG. 4 shows additional compounds that were tested for activity.

### SYNTHESIS OF COMPOUNDS

The compounds of this invention can be synthesized by following the published general procedures (e.g. Sun et al., 2003, J. Med. Chem., 46:1116-119). But the following intermediates are specific to compounds of this invention and may be used in place of their respective counterparts in the above-mentioned general procedure: 4,5-difluoro-oxindole; (4R,6R)-t-butyl-6-(2-aminoethyl)-2,2-dimethyl-1,3-dioxane-4-acetate; t-Butyl(3R,5S)-6-hydroxy-3,5-O-isopropylidene-3,5-dihydroxyhexanoate, and 4-amino-3-hydroxy-butanic acid. These intermediates may be purchased from commercial sources (e.g. Fisher Scientific, Fairlawn, N.J., or Kaneka Corp., Japan). Another variation from the above-mentioned general procedure is that in the synthesis of 1/1a and 2/2a using (4R,6R)-t-butyl-6-(2-aminoethyl)-2,2-dimethyl-1,3-dioxane-4-acetate, the protecting groups need to be removed from the final product. Yet another variation from the above-mentioned general procedure is that in the synthesis of 3 and 4 using 4-amino-3-hydroxy-butanic acid, the acid needs to be protected before amidation and the protection group needs to be removed from the final product. These variations from the above-mentioned general procedure can be understood and carried out by those skilled in the art. Thus, the compounds of the present invention can be synthesized by those skilled in the art.

The compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

### EXAMPLE 1

### (3R,5R)-7-{[5-(5-Fluoro-2-oxo-1,2-dihydroindol-3-ylidenemethyl)-2,4-dimethyl-1H-pyrrole-3-carbonyl]-amino}-3,5-dihydroxyheptanoic acid, sodium salt

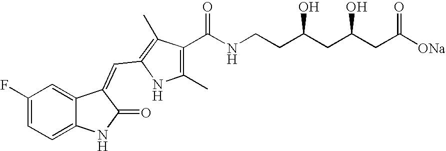 The synthesis of the title compound is summarized in Scheme 1. In the first step, 5-fluoro-1,3-dihydroindol-2-one (1A, purchased from Combi-Blocks, Inc.) was condensed with 5-formyl-2,4-dimethyl-1H-pyrrole-3-carboxylic acid in refluxing ethanol under the influence of catalytic amounts of pyrrolidine in analogy to the literature-known preparation of similar compounds (Li Sun, Chris Liang, Sheri Shirazian, Yong Zhou, Todd Miller, Jean Cui, Juri Y. Fukuda, Ji-Yu Chu, Asaad Nematalla, Xueyan Wang, Hui Chen, Anand Sistla, Tony C. Luu, Flora Tang, James Wei, and Cho Tang. Discovery of 5-[5-Fluoro-2-oxo-1,2-dihydroindol-(3Z)-ylidenemethyl]-2,4-dimethyl-1H-pyrrole-3-carboxylic Acid (2-Diethylaminoethyl)amide, a Novel Tyrosine Kinase Inhibitor Targeting Vascular Endothelial and Platelet-Derived Growth Factor Receptor Tyrosine Kinase. J. Med. Chem. 2003, 46, 1116-1119) to give pyrrole carboxylic acid 1B in 92% yield. 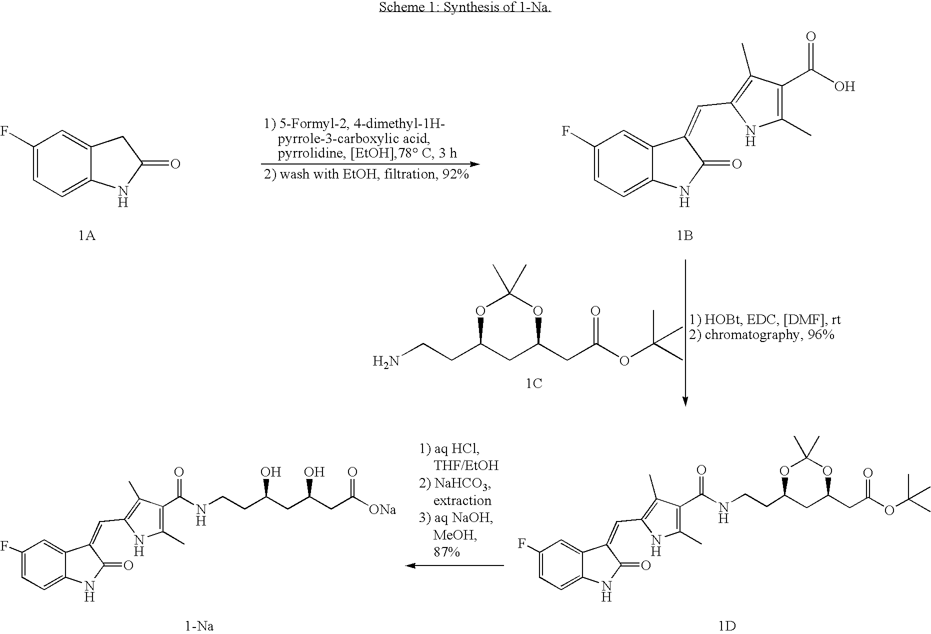
Amide coupling between carboxylic acid 1B and amine 1C (obtained from Acros) was affected by treatment with hydroxybenzotriazole, 1-(3-dimethylaminopropyl-3-ethylcarbodiimide hydrochloride, and triethylamine in DMF to afford 1D, after chromatographic purification, in 96% yield. Removal of the acetonide and tert-butyl ester protective groups was then conducted in a stepwise fashion (H. Jendralla, E. Granzer, B. Von Kerekjarto, R. Krause, U. Schacht, E. Baader, W. Bartmann, G. Beck, A. Bergmann, and et al. Synthesis and biological activity of new HMG-CoA reductase inhibitors. 3. Lactones of 6-phenoxy-3,5-dihydroxyhexanoic acids. J. Med. Chem. 1991, 34, 2962-2983). First, the acetonide protection in 1D was removed by treatment with aqueous HCl in a mixture of THF and ethanol to give an intermediary ester diol (not shown), which was isolated by extraction after neutralization of the reaction mixture with sodium bicarbonate. This intermediate was then treated with aqueous NaOH (1 equiv) in methanol to furnish the title compound: (3R,5R)-7-{[5-(5-Fluoro-2-oxo-1,2-dihydroindol-3-ylidenemethyl)-2,4-dimethyl-1H-pyrrole-3-carbonyl]-amino}-3,5-dihydroxyheptanoic acid, sodium salt (87% yield over both steps) after concentration of the reaction mixture as a yellow solid.

### Preparation of 1B: 5-(5-Fluoro-2-oxo-1,2-dihydroindol-3-ylidenemethyl)-2,4-dimethyl-1H-pyrrole-3-carboxylic acid

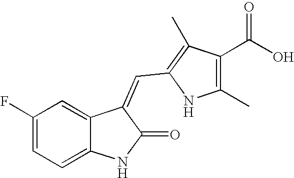 A mixture of 5-fluoro-1,3-dihydroindol-2-one (0.81 g, 5.1 mmol), 5-formyl-2,4-dimethyl-1H-pyrrole-3-carboxylic acid (0.98 g, 5.35 mmol), pyrrolidine (6 drops) and absolute ethanol (15 mL) was heated to reflux for 3 hours. The mixture was cooled to room temperature and the solids were collected by filtration. The solids were stirred with ethanol (14 mL) at 72° C. for 30 minutes. The mixture was cooled to room temperature. The solids were collected by filtration, washed with ethanol (3 mL), dried under vacuum at 54° C. overnight to give 5-(5-fluoro-2-oxo-1,2-dihydroindol-3-ylidenemethyl)-2,4-dimethyl-1H-pyrrole-3-carboxylic acid (1.4 g, 91.5% yield) as an orange solid. ¹H NMR (300 MHz, DMSO-d₆) δ 12.19 (br s, 1H), 10.95 (s, 1H), 7.90-7.70 (m, 2H), 7.00-6.80 (m, 2H), 2.54 (s, 3H), 2.51 (s, 3H). ¹³C NMR (75 MHz, DMSO-d₆) δ 169.4, 165.7, 159.6, 156.5, 140.7, 134.6, 133.3, 128.9, 126.8, 125.9, 124.7, 115.5, 114.2, 110.9, 110.0, 106.3, 105.9, 14.6, 11.6.

### Preparation of 1D: (4R,6R)-[6-(2-{[5-(Fluoro-2-oxo-1,2-dihydroindol-3-ylidenemethyl)-2,4-dimethyl-1H-pyrrole-3-carbonyl]-amino}-ethyl)-2,2-dimethyl-[1,3]dioxan-4-yl]-acetic acid tert-butyl ester

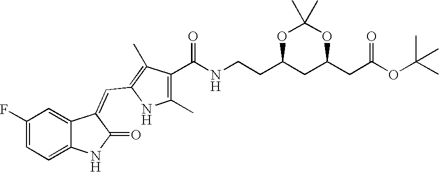 To a stirred solution of 5-(5-fluoro-2-oxo-1,2-dihydroindol-3-ylidenemethyl)-2,4-dimethyl-1H-pyrrole-3-carboxylic acid (1.3 g, 4.33 mmol) in dimethylformamide (11.6 mL) at room temperature were added 1-(3-dimethylaminopropyl-3-ethylcarbodiimide hydrochloride (1.25 g, 6.39 mmol); hydroxybenzotriazole (0.88 g, 6.39 mmol), triethylamine (1.3 mL, 9.34 mmol), and (4R,6R)-[6-(2-aminoethyl)-2,2-dimethyl-[1,3]dioxan-4-yl]-acetic acid tert-butyl ester (1.38 g, 4.87 mmol). The reaction mixture was stirred at room temperature for 30 h, then filtered through a silica gel pad and washed with ethyl acetate (100 mL). The filtrate was concentrated and the residue was diluted with water (20 mL), saturated sodium bicarbonate solution (10 mL) and 10 N sodium hydroxide solution (5 mL). The mixture was extracted with a mixture of methylene chloride/methanol (9/1, 2×50 mL). The combined organic layers were concentrated to dryness. The residue was triturated with heptane/diethyl ether (3/1, 60 mL). The solids were collected by filtration and dried under vacuum at 34° C. overnight to obtain (4R,6R)-[6-(2-{[5-(fluoro-2-oxo-1,2-dihydro-indol-3-ylidenemethyl)-2,4-dimethyl-1H-pyrrole-3-carbonyl]-amino}ethyl)-2,2-dimethyl-[1,3]dioxan-4-yl]-acetic acid tert-butyl ester (2.3 g, 95.6%) as a yellow solid. ¹H NMR (300 MHz, DMSO-d₆) δ 11.05 (br s, 1H), 7.94 (d, J=6.9 Hz, 1H), 7.85 (s, 1H), 7.14-6.90 (m, 2H), 4.35 (m, 1H), 4.12 (m, 1H), 3.51 (br s, 1H), 3.42 (m, 2H), 2.64 (m, 2H), 2.57 (s, 3H), 2.56 (s, 3H), 2.50-2.30 (m, 2H), 1.76 (m, 3H), 1.54 (s, 9H), 1.41 (s, 3H), 1.24 (m, 1H). ¹³C NMR (75 MHz, DMSO-d₆) δ 169.4, 164.4, 159.6, 156.5, 136.2, 134.3, 129.9, 127.1, 126.9, 125.6, 124.7, 120.8, 114.4, 112.3, 112.0, 109.9, 109.8, 105.9, 105.6, 97.9, 79.6, 66.5, 65.9, 42.2, 35.9, 35.8, 35.1, 29.9, 27.7, 19.6, 13.3, 10.5.

### Preparation of 1-Na: (3R,5R)-7-{[5-(5-Fluoro-2-oxo-1,2-dihydroindol-3-ylidenemethyl)-2,4-dimethyl-1H-pyrrole-3-carbonyl]-amino}-3,5-dihydroxyheptanoic acid, sodium salt

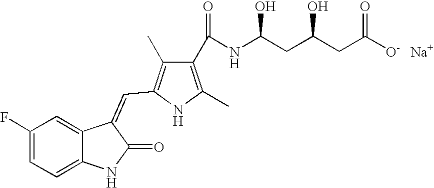 Under argon atmosphere and exclusion of light, a solution of (4R,6R)-[6-(2-{[5-(fluoro-2-oxo-1,2-dihydroindol-3-ylidenemethyl)-2,4-dimethyl-1H-pyrrole-3-carbonyl]-amino}-ethyl)-2,2-dimethyl-[1,3]dioxan-4-yl]-acetic acid tert-butyl ester (1.69 g, 3.04 mmol) in ethanol (15.2 mL), THF (7.6 mL) and 2 N hydrochloric acid (1.7 mL) was stirred at room temperature for 24 hours. The reaction mixture was neutralized with sodium bicarbonate solution (0.256 g NaHCO₃ in 5 mL water) to pH 7 and concentrated to remove ethanol and THF. The residue was diluted with water (50 mL) and extracted with a mixture of methyl tert-butyl ether/methanol (9/1, 200 mL), and then with methyl tert-butyl ether (3×50 mL). The combined organic layers were dried over magnesium sulfate and concentrated to dryness to give (3R,5R)-7-{[5-(5-fluoro-2-oxo-1,2-dihydroindol-3-ylidenemethyl)-2,4-dimethyl-1H-pyrrole-3-carbonyl]-amino}-3,5-dihydroxyheptanoic acid tert-butyl ester (1.57 g, 3 mmol). This ester (1.56 g, 3.0 mmol) was dissolved in methanol (33.4 mL) and a solution of sodium hydroxide (0.12 g, 3.0 mmol) in deionized water (8.3 mL) was added. The mixture was stirred at room temperature for 3 hours. The reaction mixture was concentrated to dryness. The residue was dissolved in methanol (66 mL) and the mixture was concentrated again. The mixture was triturated with isopropanol (40 mL). The solids were collected by filtration, washed with diethyl ether (100 mL) and dried under vacuum at 34° C. for 3 hours to furnish (3R,5R)-7-{[5-(5-fluoro-2-oxo-1,2-dihydroindol-3-ylidenemethyl)-2,4-dimethyl-1H-pyrrole-3-carbonyl]-amino}-3,5-dihydroxyheptanoic acid, sodium salt (1.28 g, 87.4% yield over two steps) as a yellow solid. Mp 256-258° C. (decomposition). ¹H NMR (300 MHz, methanol-d₄) δ 7.49 (s, 1H), 7.31 (d, J=8.4 Hz, 1H), 6.74 (d, J=6.6 Hz, 1H), 4.03 (m, 1H), 3.83 (m, 1H), 3.45 (m, 1H), 3.37 (m, 1H), 2.38 (s, 3H), 2.34 (s, 3H), 2.25 (m, 2H), 1.85-1.40 (m, 4H). ¹³C NMR (75 MHz, methanol-d₄) δ 180.1, 171.4; 168.4, 161.8, 158.7, 137.7, 135.7, 131.4, 128.6, 128.5, 127.3, 125.2, 121.1, 116.4, 113.6, 113.3, 111.1, 110.9, 106.3, 106.0, 69.1, 68.9, 45.5, 44.9, 37.8, 37.7, 13.4, 10.8.

### EXAMPLE WITH α-SUBSTITUENT

### 2-Ethyl-4-({5-[5-fluoro-2-oxo-1,2-dihydro-indol-(3Z)-ylidenemethyl]-2,4-dimethyl-1H-pyrrole-3-carbonyl}-amino)-3-hydroxy-butyric acid

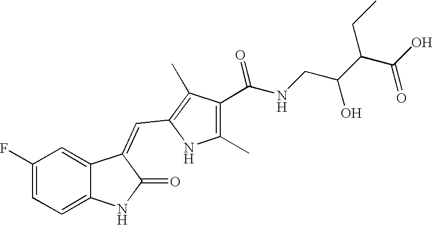 The advanced intermediate 4-Amino-2-ethyl-3-hydroxy-butyric acid ethyl ester can be made following published procedures (e.g. Seebach, Dieter; Chow, Hak-Fun; Jackson, Richard F. W.; Lawson, Kevin; Sutter, Marius A.; et al.; J. Am. Chem. Soc. 1985, 107, 18, 5292-5293. Itoh, Toshiyuki; Takagi, Yumiko; Fujisawa, Tamotsu; Tetrahedron Lett. 1989, 30; 29, 3811-3812). Subsequent amide coupling with 1B followed by deprotection afforded the title compound.

### EXAMPLE 2

### (3R,5R)-7-({5-[4,5-Difluoro-2-oxo-1,2-dihydro-indol-(3Z)-ylidenemethyl]-2,4-dimethyl-1H-pyrrole-3-carbonyl}-amino)-3,5-dihydroxy-heptanoic acid, sodium salt

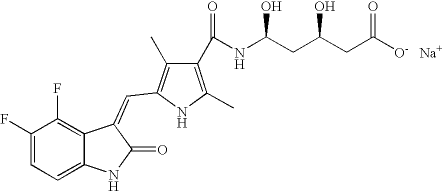 The title compound was prepared following the procedure described in the preparation of Example 1. In this synthesis, 4,5-difluoro-1,3-dihydroindol-2-one was used instead of 5-fluoro-1,3-dihydroindol-2-one as in Example 1. LC-MS: a single peak was observed at 254 nm, MH⁺ calcd for the free acid C₂₃H₂₅F₂N₃O₆: 478, obtained 478. ¹H NMR (400 MHz, methanol-d₄) δ 7.71 (d, J=2.4 Hz, 1H), 7.00 (m, 1H), 6.65 (dd, J=3.2 Hz, J=8.4 Hz, 1H), 4.13 (m, 1H), 3.93 (m, 1H), 3.56 (m, 1H), 3.45 (m, 1H), 2.48 (s, 3H), 2.39 (s, 3H), 2.34 (m, 2H), 1.84 (m, 1H), 1.69 (m, 3H).

### EXAMPLE 3

### 4-({5-[5-Fluoro-2-oxo-1,2-dihydro-indol-(3Z)-ylidenemethyl]-2,4-dimethyl-1H-pyrrole3-carbonyl}-amino)-3-hydroxy-butyric acid

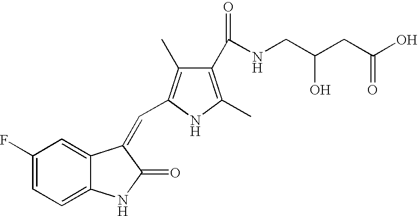

The title compound was prepared following the procedure described in the preparation of Example 1. In this synthesis, 4-amino-3-hydroxybutanoic acid was used instead of (4R,6R)-[6-(2-aminoethyl)-2,2-dimethyl-[1,3]dioxan-4-yl]-acetic acid tert-butyl ester as in Example 1. LC-MS: a single peak was observed at 254 nm, MH⁺ calcd for the free acid C₂₀H₂₀FN₃O₅: 402, obtained 402. ¹H NMR (400 MHz, DMSO-d₆) δ 13.68 (s, 1H), 11.40 (s, 1H), 10.90 (s, 1H), 7.76 (dd, J=3.2 Hz, J=8.4 Hz, 1H), 7.71 (s, 1H), 7.59 (t, J=4.8 Hz, 1H), 6.92 (m, 1H), 6.83 (dd, J=4.8 Hz, J=9.2 Hz, 1H), 4.00 (m, 1H), 3.33 (m, 2H, buried in water signals), 3.24 (m, 2H), 2.43 (s, 3H), 2.41 (s, 3H).

### EXAMPLE 4

### 4-({5-[4,5-Difluoro-2-oxo-1,2-dihydro-indol-(3Z)-ylidenemethyl]-2,4-dimethyl-1H-pyrrole-3-carbonyl}-amino)-3-hydroxy-butyric acid

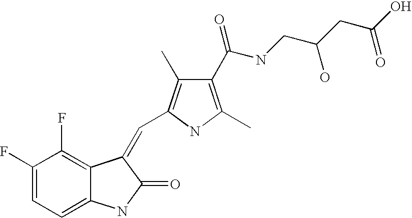 The title compound was prepared following the procedure described in the preparation of Example 1. In this synthesis, 4-amino-3-hydroxylbutanoic acid was used instead of (4R,6R)-[6-(2-aminoethyl)-2,2-dimethyl-[1,3]dioxan-4-yl]-acetic acid tert-butyl ester as in Example 1. LC-MS: a single peak was observed at 254 nm, MH⁺ calcd for the free acid C₂₀H₁₉F₂N₃O₅: 420, obtained 420. ¹H NMR (400 MHz, DMSO-d₆) δ 13.55 (s, 1H), 12.10 (s, 1H), 11.15 (s, 1H), 7.67 (t, J=6.0 Hz, 1H), 7.59 (d, J=2.0 Hz, 1H), 7.14 (m, 1H), 6.68 (dd, J=3.2 Hz, J=8.4 Hz, 1H), 5.05 (b, 1H), 4.03 (m, 1H), 3.31 (m, 2H), 3.25 (m, 2H), 2.44 (s, 3H), 2.32 (s, 3H).

### EXAMPLE 5

### (3R,5S)-6-({5-[5-Fluoro-2-oxo-1,2-dihydro-indol-(3Z)-ylidenemethyl]-2,4-dimethyl-1H-pyrrole-3-carbonyl}-amino)-3,5-dihydroxy-hexanoic acid, sodium salt

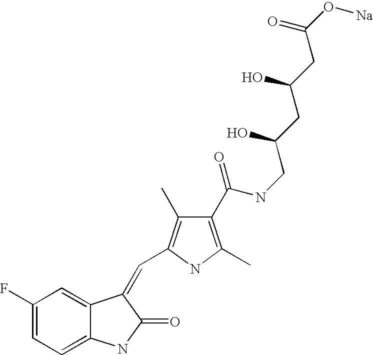

Preparation of ((4R,6S)-6-Aminomethyl-2,2-dimethyl-[1,3]dioxan-4-yl)-acetic acid tert-butyl ester: Triflic anhydride 1.4 mL (2.36 g, 8.345 mmol) was dropwise added at −78° C. to a solution of 2,6-lutidine 1.35 mL (11.63 mmol) and t-Butyl-(3R,5S)-6-hydroxy-3,5-O-isopropylidene-3,5-dihydroxy-hexanoate 1.981 g (7.609 mmol, obtained from Kaneka Corp.) in dichloromethane (anh., 50 mL) over 3 minutes. The mixture was stirred at −78° C. for 10 min, then placed on ice-slush bath and stirred at 0° C. for 45 min. The resulting pink mixture was transferred into ice-cooled solution of ammonia in methanol (7 M solution, 200 mL). The mixture was placed on ambient water bath and stirred at RT for 6 hours. The reaction mix was evaporated to dryness, the residue partitioned between ether (200 mL) and aqueous potassium carbonate (6 g in 200 mL of water), the aqueous phase re-extracted with ether (150 mL). Combined organic extracts were dried (magnesium sulfate) and evaporated. The crude product was purified on a column of silica (125 g) eluting with a mix of chloroform-methanol-conc. aq. ammonia 100:10:1 (v/v) (1.5 L) to give Y=1.777 g of a colorless liquid (90%), ((4R,6S)-6-Aminomethyl-2,2-dimethyl-[1,3]dioxan-4-yl)-acetic acid tert-butyl ester.

¹H (^{d}DMSO, 400 MHz): δ 4.167 (m, 1H), 3.741 (m, 1H), 2.484 (m, 2H), 2.384 (ddAB, J=15.2 Hz, 5.1 Hz, 1H), 2.201 (ddAB, J=15 Hz, 7.8 Hz, 1H), 1.533 (br d, J=12.5 Hz, 1H), 1.373 (s, 9H), 1.363 (s, 3H), 1.250 (br s, 2H), 1.223 (s, 3H).

### Preparation of (3R,5S)-6-({5-[5-Fluoro-2-oxo-1,2-dihydro-indol-(3Z)-ylidenemethyl]-2,4-dimethyl-1H-pyrrole-3-carbonyl}-amino)-3,5-dihydroxy-hexanoic acid, sodium salt

5-[(Z)-(5-fluoro-2-oxo-1,2-dihydro-3H-indol-3-ylidene)methyl]-2,4-dimethyl-1H-pyrrole-3-carboxylic acid 1-oxy-7-azabenztriazole ester 419 mg (1.00 mmol, prepared according to U.S. Pat. No. 6,653,308) was suspended in anh. dimethylacetamide (4 mL) and a solution of ((4R,6S)-6-Aminomethyl-2,2-dimethyl-[1,3]dioxan-4-yl)-acetic acid tert-butyl ester 310 mg (1.2 mmol) and diisopropylethyl amine 175 μL (1.0 mmol) in anh. DMAc (7 mL) was added to the slurry. The mixture was stirred for 20 min at RT. The obtained homogenous mixture was evaporated under high vacuum (0.5 Torr, 45° C.). The residue was taken up with methanol 10 mL, sonicated for 2 minutes, then allowed to crystallize at 5° C. for 3 hours. The precipitated intermediate (acetonide-tBu ester) was collected by filtration, washed with ice-cold methanol and dried under high vacuum. This intermediate (485 mg of an orange-yellow cryst. solid, 89.5% th.) was dissolved in neat TFA 20 mL and the obtained solution was kept at RT for 15 min, then evaporated. The residue was dried under high vacuum for 1 day. The residue was dissolved in a mixture of methanol 100 mL and THF 100 mL (with 15 min stirring). 40 mL of 1M NaOH was added and the mixture was kept at RT for 30 min. The mixture was acidified with 2M HCl to pH=3. The mixture was concentrated to a small volume on rotavap to remove organic solvents, the precipitate was collected by filtration, washed with water and dried by suction, then under high vacuum. This precipitate (consisting of the free acid with approx 5% of the corresponding lactone) was dissolved in a mixture of methanol (200 mL), water (30 mL) and 1M NaOH (0.96 mL) with stirring and gentle heating to reflux for 3 minutes. The mixture was stirred at RT for additional 15 minutes, then saturated with CO₂ (g), evaporated to dryness and dried under high vacuum to give Y=376.5 mg (90%) of an orange solid, (3R,5S)-6-({5-[5-Fluoro-2-oxo-1,2-dihydro-indol-(3Z)-ylidenemethyl]-2,4-dimethyl-1H-pyrrole-3-carbonyl}-amino)-3,5-dihydroxy-hexanoic acid, sodium salt. LC/MS(+ESI): 446 (M+1)

¹H (D₂O, 400 MHz): 6.655 (br d, J=9.4 Hz, 1H), 6.594 (m, 2H), 6.292 (dd, J=8.2 Hz, 4.7 Hz, 1H), 4.155 (m, 1H), 3.891 (m, 1H), 3.405 (dd, J=14.1 Hz, 3.9 Hz, 1H), 3.195 (dd, J=15.7 Hz, 7.5 Hz, 1H), 2.429 (ddAB, J=14.9 Hz, 5.0 Hz, 1H), 2.329 (ddAB, J=14.9 Hz, 8.2 Hz, 1H), 1.782 (m, 2H).

### EXAMPLES 6-8

The general procedure for the synthesis of amides of Examples 3 and 4 is shown in Scheme 2 below: 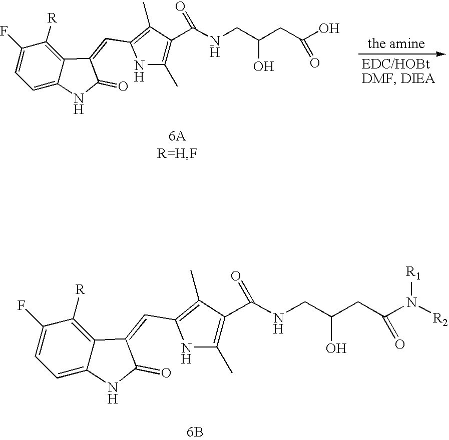
A corresponding amine (0.3 mmol) was added to a solution of compound 6A (80 mg, 0.2 mmol), EDC (0.25 mmol), HOBt (0.25 mmol), and DIEA (1 mmol) in DMF (3 mL). After the solution was stirred at 25° C. overnight, DMF was removed via evaporation under reduced pressure. The resulting residue was suspended in ethyl acetate (200 mL), washed by saturated NaHCO₃ (3×) and brine (3×), and dried over Na₂SO₄. The ethyl acetate was removed under vacuum to give the crude product. This crude material was subjected to preparative HPLC to give the final product 6B, which was subsequently characterized by LC-MS and NMR spectroscopy.

### EXAMPLE 6

### 5-[4,5-Difluoro-2-oxo-1,2-dihydro-indol-(3Z)-ylidenemethyl]-2,4-dimethyl-1H-pyrrole-3-carboxylic acid (2-hydroxy-4-morpholin-4-yl-4-oxo-butyl)-amide

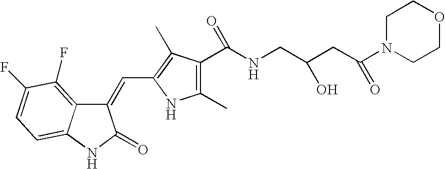 Preparative HPLC gave 70 mg of the title compound (75%). LC-MS: single peak at 254 nm, MH⁺ calcd. for C₂₄H₂₆F₂N₄O₅: 489, obtained: 489. ¹H-NMR (DMSO-d₆, 400 MHz), δ 13.55 (s, 1H), 11.20 (s, 1H), 7.64 (t, J=6.0 Hz, 1H), 7.58 (d, J=2.4 Hz, 1H), 7.13 (m, 1H), 6.70 (dd, J=3.2 Hz, J=8.4 Hz, 1H), 4.99 (s, 1H), 4.04 (m, 1H), 3.20-3.60 (m, 12H), 2.45 (s, 3H), 2.32 (s, 3H).

### EXAMPLE 7

### 5-[5-Fluoro-2-oxo-1,2-dihydro-indol-(3Z)-ylidenemethyl]-2,4-dimethyl-1H-pyrrole-3-carboxylic acid (2-hydroxy-4-morpholin-4-yl-4-oxo-butyl)-amide

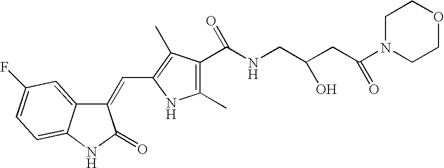 Preparative HPLC gave 50 mg of the title compound (53%). LC-MS: single peak at 254 nm, MH⁺ calcd. for C₂₄H₂₇FN₄O₅: 471, obtained: 471. ¹H-NMR (DMSO-d₆, 400 MHz), δ 13.69 (s, 1H), 10.91 (s, 1H), 7.76 (dd, J=3.2 Hz, J=9.2 Hz, 1H), 7.71 (s, 1H), 7.57 (t, J=6.0 Hz, 1H), 6.95 (m, 1H), 6.83 (dd, J=4.8 Hz, J=8.8 Hz, 1H), 4.98 (d, J=5.2 Hz, 1H), 4.04 (m, 1H), 3.53 (m, 5H), 3.45 (m, 4H), 3.28 (m, 3H), 2.43 (s, 3H), 2.41 (s, 3H).

### EXAMPLE 8

### 5-[5-Fluoro-2-oxo-1,2-dihydro-indol-(3Z)-ylidenemethyl]-2,4-dimethyl-1H-pyrrole-3-carboxylic acid [2-hydroxy-4-(4-methyl-piperazin-1-yl)-4-oxo-butyl]-amide

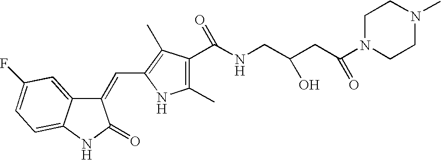 Preparative HPLC gave 55 mg of the title compound (57%). LC-MS: single peak at 254 nm, MH⁺ calcd. for C₂₅H₃₀FN₅O₄: 484, obtained: 484. ¹H-NMR (DMSO-d₆, 400 MHz), δ 13.65 (s, 1H), 10.90 (s, 1H), 7.74 (m, 2H), 7.71 (m, 1H), 7.54 (m, 1H), 6.92 (m, 1H), 6.83 (m, 1H), 4.95 (s, 1H), 4.04 (m, 1H), 3.44 (m, 4H), 3.25 (m, 4H, buried in water signals), 2.43 (s, 3H), 2.41 (s, 3H), 2.25 (m, 4H), 2.16 (s, 3H).

### EXAMPLES 9-16

The general procedure for the synthesis of amides of Examples 1 and 5 is shown in Scheme 3 below: 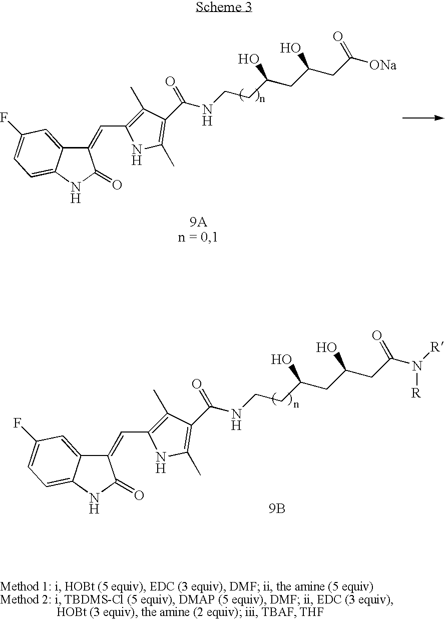
Method 1: EDC (1 mmol), and HOBt (0.6 mmol) were added to a solution of compound 9A (0.2 mmol) in DMF (3 mL). After the solution was stirred at 25° C. for 3 h, the corresponding amine (1.0 mmol) was added, and the solution was stirred at 25° C. overnight. If the reaction was not complete, the solution was stirred at 50° C. for another couple of hours. This DMF solution was directly subjected to preparative HPLC to obtain the final product 9B, which was subsequently characterized by LC-MS and proton NMR spectroscopy.
Method 2: TBDMS-Cl (1.0 mmol), and DMAP (1.0 mmol) were added to a solution of compound 9A (0.2 mmol) in DMF (3 mL). After the solution was stirred at 25° C. for 5 h (LC-MS demonstrated that a mixture of mono- and disilyl ether products was formed), EDC (1 mmol), HOBt (0.6 mmol), and the corresponding amine (0.4 mmol) were added to the solution. The solution was continuously stirred at 25° C. overnight (LC-MS demonstrated that a mixture of the amides of the corresponding mono- and di-silyl ether products was formed). After the solvent was removed via evaporation under reduced pressure, the resulting residue was suspended in ethyl acetate (100 mL), washed with saturated NaHCO₃ (3×), and brine (3×). The organic solvent was then evaporated under vacuum to give the crude silyl ether amide products. TBAF (3 equiv, 1M in THF) was added to a solution of this crude material in THF. After stirring at 25° C. for 30 min., the THF was removed under reduced pressure. The residue was suspended in ethyl acetate (100 mL), washed with brine (3×). The organic solvent was then evaporated under reduced pressure, and the resulting residue was directly subjected to preparative HPLC to obtain the final product 9B, which was subsequently characterized by LC-MS and proton NMR spectroscopy.

### EXAMPLE 9

### 5-[5-Fluoro-2-oxo-1,2-dihydro-indol-(3Z)-ylidenemethyl]-2,4-dimethyl-1H-pyrrole-3-carboxylic acid ((3R,5R)-6-dimethylcarbamoyl-3,5-dihydroxy-hexyl)-amide

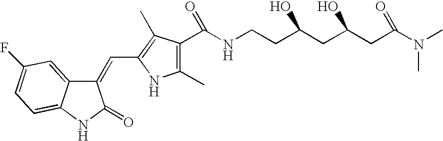 This compound was prepared via Method 2. An amount of 65 mg (64%) product was obtained after preparative HPLC. LC-MS: single peak at 254 nm, MH⁺ calcd. for C₂₅H₃₁FN₄O₅: 487, obtained: 487. ¹H-NMR (DMSO-d₆, 400 MHz), δ 13.67 (s, 1H), 10.90 (s, 1H), 7.76 (dd, J=2.8 Hz, J=9.2 Hz, 1H), 7.71 (s, 1H), 7.63 (t, J=5.6 Hz, 1H), 6.92 (m, 1H), 6.83 (dd, J=4.8 Hz, J=8.8 Hz, 1H), 4.72 (d, J=4.4 Hz, 1H), 4.67 (d, J=4.8 Hz, 1H), 4.00 (m, 1H), 3.71 (m, 1H), 3.31 (m, 2H), 2.96 (s, 3H), 2.80 (s, 1H), 2.42 (s, 3H), 2.40 (s, 3H), 2.39 (m, 2H), 1.65 (m, 1H), 1.52 (m, 3H).

### EXAMPLE 10

### 5-[5-Fluoro-2-oxo-1,2-dihydro-indol-(3Z)-ylidenemethyl]-2,4-dimethyl-1H-pyrrole-3-carboxylic acid ((3R,5R)-3,5-dihydroxy-7-oxo-7-pyrrolidin-1-yl-heptyl)-amide

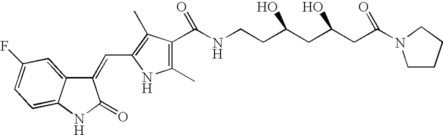 This compound was prepared via Method 1. An amount of 55 mg (61%) product was obtained after preparative HPLC. LC-MS: single peak at 254 nm, MH⁺ calcd. for C₂₇H₃₃FN₄O₅: 513, obtained: 513. ¹H-NMR (DMSO-d₆, 400 MHz), δ 13.66 (s, 1H), 10.90 (s, 1H), 7.76 (dd, J=2.4 Hz, J=9.2 Hz, 1H), 7.71 (s, 1H), 7.63 (t, J=5.6 Hz, 1H), 6.91 (m, 1H), 6.83 (dd, J=4.8 Hz, J=8.8 Hz, 1H), 4.75 (d, J=4.4 Hz, 1H), 4.68 (d, J=4.8 Hz, 1H), 4.03 (m, 1H), 3.70 (m, 1H), 3.40 (m, 2H), 3.26 (m, 4H), 2.42 (s, 3H), 2.40 (s, 3H), 2.34 (m, 2H), 1.83 (m, 2H), 1.74 (m, 2H), 1.65 (m, 1H), 1.52 (m, 3H).

### EXAMPLE 11

### 5-[5-Fluoro-2-oxo-1,2-dihydro-indol-(3Z)-ylidenemethyl]-2,4-dimethyl-1H-pyrrole-3-carboxylic acid ((3R,5R)-3,5-dihydroxy-7-morpholin-4-yl-7-oxo-heptyl)-amide

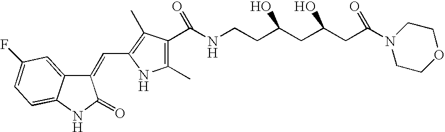 This compound was prepared via Method 2. An amount of 72 mg (66%) product was obtained after preparative HPLC. LC-MS: single peak at 254 nm, MH⁺ calcd. for C₂₇H₃₃FN₄O₆: 529, obtained: 529. ¹H-NMR (DMSO-d₆, 400 MHz), δ 13.66 (s, 1H), 7.76 (dd, J=2.4 Hz, J=9.2 Hz, 1H), 7.71 (s, 1H), 7.63 (t, J=5.6 Hz, 1H), 6.91 (m, 1H), 6.83 (dd, J=4.8 Hz, J=8.8 Hz, 1H), 6.70 (b, 1H), 4.71 (d, J=4.4 Hz, 1H), 4.67 (d, J=4.8 Hz, 1H), 4.01 (m, 1H), 3.70 (m, 1H), 3.51 (m, 5H), 3.45 (m, 3H), 3.42-3.24 (m, 4H), 2.42 (s, 3H), 2.40 (s, 3H), 1.65 (m, 1H), 1.52 (m, 3H).

### EXAMPLE 12

### 5-[5-Fluoro-2-oxo-1,2-dihydro-indol-(3Z)-ylidenemethyl]-2,4-dimethyl-1H-pyrrole-3-carboxylic acid [(3R,5R)-3,5-dihydroxy-7-(4-methyl-piperazin-1-yl)-7-oxo-heptyl]-amide

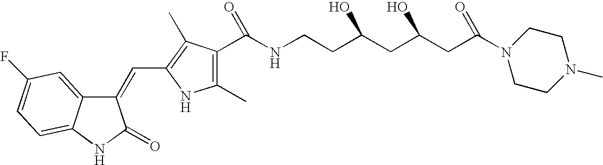 This compound was prepared via Method 2. An amount of 30 mg (27%) product was obtained after preparative HPLC. LC-MS: single peak at 254 nm, MH⁺ calcd. for C₂₈H₃₆FN₅O₅: 542, obtained: 542. ¹H-NMR (DMSO-d₆, 400 MHz), δ 13.66 (s, 1H), 7.76 (dd, J=2.4 Hz, J=9.2 Hz, 1H), 7.70 (s, 1H), 7.63 (t, J=5.6 Hz, 1H), 6.91 (m, 1H), 6.84 (dd, J=4.8 Hz, J=8.8 Hz, 1H), 4.70 (b, 2H), 4.01 (m, 1H), 3.70 (m, 1H), 3.43 (m, 4H), 3.30 (m, 4H), 2.42 (s, 3H), 2.40 (s, 3H), 2.26 (m, 2H), 2.21 (m, 2H), 2.15 (s, 3H), 1.65 (m, 1H), 1.52 (m, 3H).

### EXAMPLE 13

### 5-[5-Fluoro-2-oxo-1,2-dihydro-indol-(3Z)-ylidenemethyl]-2,4-dimethyl-1H-pyrrole-3-carboxylic acid ((2S,4R)-2,4-dihydroxy-6-oxo-6-pyrrolidin-1-yl-hexyl)-amide

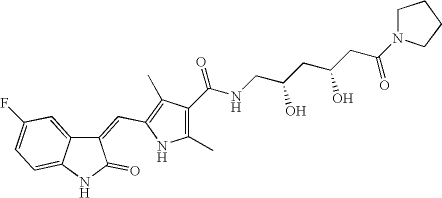 This compound was prepared via Method 1. An amount of 66 mg (54%) product was obtained after preparative HPLC. LC-MS: single peak at 254 nm, MH⁺ calcd. for C₂₄H₂₉FN₄O₅: 473, obtained: 473. ¹H-NMR (DMSO-d₆, 400 MHz), δ 13.72 (s, 1H), 10.90 (s, 1H), 7.77 (dd, J=2.4 Hz, J=9.2 Hz, 1H), 7.72 (s, 1H), 7.49 (t, J=5.6 Hz, 1H), 6.92 (m, 1H), 6.83 (dd, J=4.4 Hz, J=8.4 Hz, 1H), 4.80 (s, 1H), 4.78 (s, 1H), 4.05 (m, 1H), 3.76 (m, 1H), 3.41 (m, 2H), 3.26 (m, 4H), 2.44 (s, 3H), 2.42 (s, 3H), 2.36 (m, 2H), 1.85 (m, 2H), 1.75 (m, 2H), 1.61 (m, 1H), 1.50 (m, 1H).

### EXAMPLE 14

### 5-[5-Fluoro-2-oxo-1,2-dihydro-indol-(3Z)-ylidenemethyl]-2,4-dimethyl-1H-pyrrole-3-carboxylic acid [(2S,4R)-2,4-dihydroxy-6-(4-methyl-piperazin-1-yl)-6-oxo-hexyl]-amide

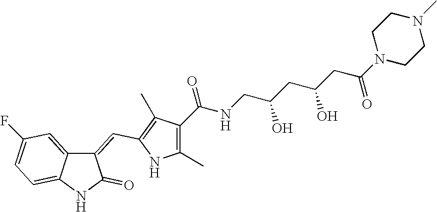 This compound was prepared via Method 2. An amount of 97 mg (75%) product was obtained after preparative HPLC. LC-MS: single peak at 254 nm, MH⁺ calcd. for C₂₇H₃₄FN₅O₅: 528, obtained: 528. ¹H-NMR (DMSO-d₆, 400 MHz), δ 13.72 (s, 1H), 10.90 (s, 1H), 7.75 (dd, J=2.4 Hz, J=9.6 Hz, 1H), 7.70 (s, 1H), 7.48 (t, J=5.6 Hz, 1H), 6.92 (m, 1H), 6.83 (dd, J=4.4 Hz, J=8.4 Hz, 1H), 4.82 (s, 1H), 4.72 (s, 1H), 4.05 (m, 1H), 3.77 (m, 1H), 3.43 (m, 4H), 3.25 (m, 2H), 3.15 (m, 4H), 2.44 (s, 3H), 2.41 (s, 3H), 2.27 (m, 2H), 2.20 (m, 2H), 2.15 (s, 3H).

### EXAMPLE 15

### 5-[5-Fluoro-2-oxo-1,2-dihydro-indol-(3Z)-yl idenemethyl]-2,4-dimethyl-1H-pyrrole-3-carboxylic acid ((3R,5R)-6-ethylcarbamoyl-3,5-dihydroxy-hexyl)-amide

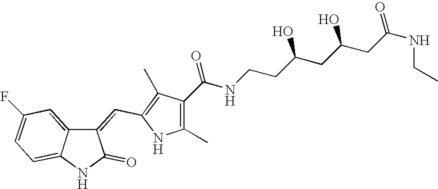 This compound was prepared via Method 2. An amount of 40 mg (40%) product was obtained after preparative HPLC. LC-MS: single peak at 254 nm, MH⁺ calcd for C₂₅H₃₁FN₄O₅: 487, obtained: 487. ¹H-NMR (DMSO-d₆, 400 MHz), δ 13.68 (s, 1H), 10.90 (s, 1H), 7.78 (t, J=5.6 Hz, 1H), 7.75 (dd, J=2.8 Hz, J=9.2 Hz, 1H), 7.70 (s, 1H), 7.61 (t, J=5.6 Hz, 1H), 6.92 (m, 1H), 6.83 (dd, J=4.8 Hz, J=8.8 Hz, 1H), 4.76 (s, 1H), 4.67 (s, 1H), 3.96 (m, 1H), 3.69 (m, 1H), 3.28 (m, 2H), 3.04 (m, 2H), 2.42 (s, 3H), 2.40 (s, 3H), 2.16 (m, 2H), 1.65 (m, 1H), 1.52 (m, 3H), 0.99 (t, 7.6 Hz, 3H).

### EXAMPLE 16

### 5-[5-Fluoro-2-oxo-1,2-dihydro-indol-(3Z)-ylidenemethyl]-2,4-dimethyl-1H-pyrrole-3-carboxylic acid ((2S,4R)-2,4-dihydroxy-6-morpholin-4-yl-7-oxo-heptyl)-amide

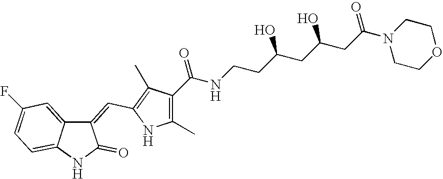 This compound was prepared via Method 2. An amount of 87 mg (69%) product was obtained after preparative HPLC. LC-MS: single peak at 254 nm, MH⁺ calcd for C₂₆H₃₁FN₄O₆: 515, obtained: 515. ¹H-NMR (DMSO-d₆, 400 MHz), δ 13.69 (s, 1H), 10.88 (s, 1H), 7.76 (dd, J=2.4 Hz, J=9.2 Hz, 1H), 7.71 (s, 1H), 7.48 (t, J=4.0 Hz, 1H), 6.91 (m, 1H), 6.83 (dd, J=4.8 Hz, J=9.2 Hz, 1H), 4.84 (d, J=4.4 Hz, 1H), 4.74 (d, J=4.4 Hz, 1H), 4.05 (m, 1H), 3.77 (m, 1H), 3.50 (m, 9H), 3.25 (m, 3H), 2.44 (s, 3H), 2.41 (s, 3H), 1.58 (m, 2H).

### EXAMPLE 17

Further amide examples of Example 1. The following examples 17a-f can be made by those skilled in the art following the above procedure and/or known procedures.

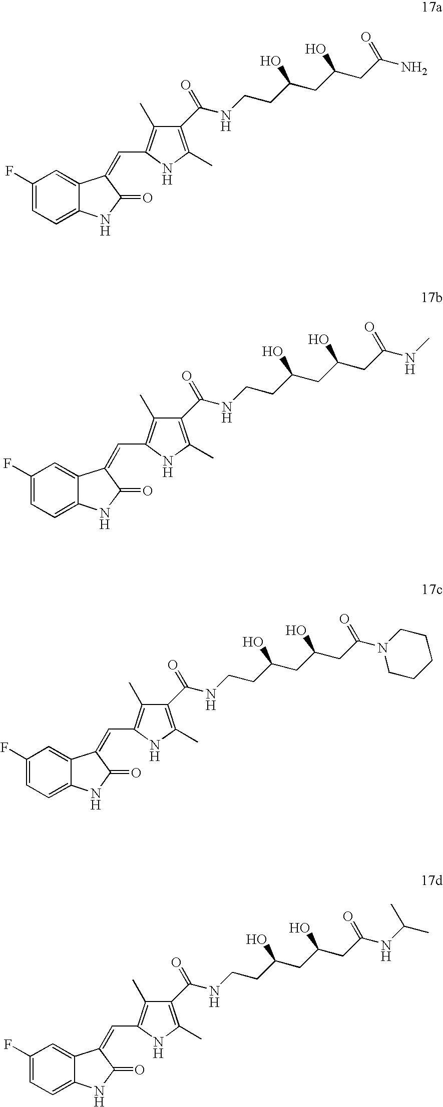

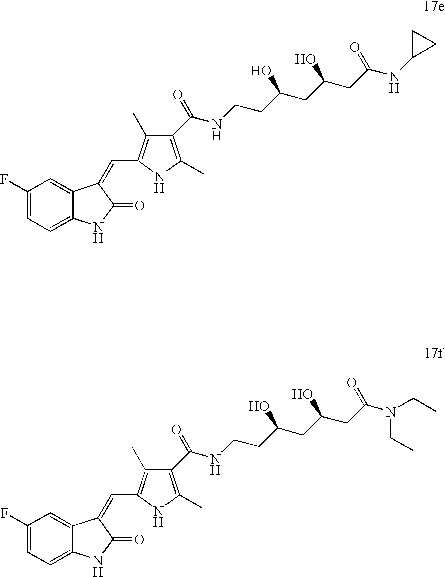

### EXAMPLE 18

Further amide examples of Example 3. The following examples 18a-f can be made by those skilled in the art following the above procedure and/or known procedures.

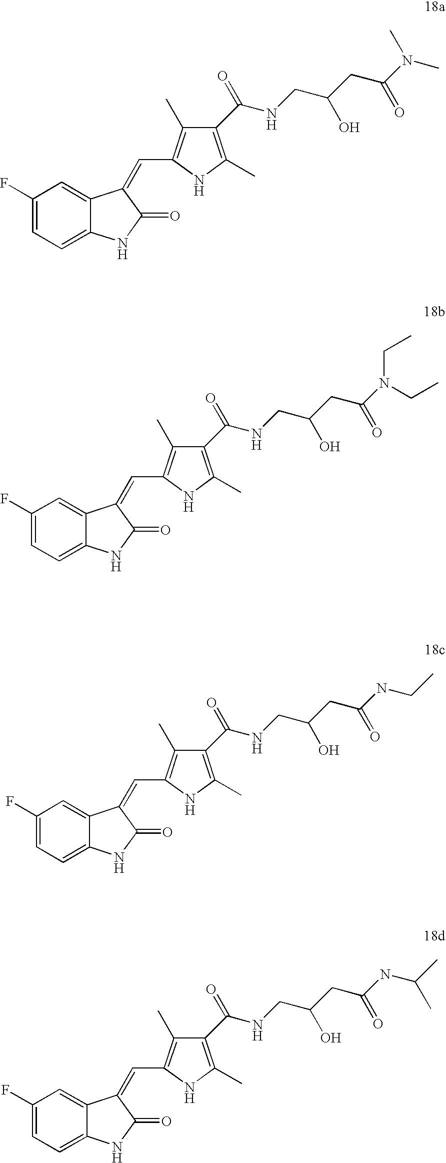

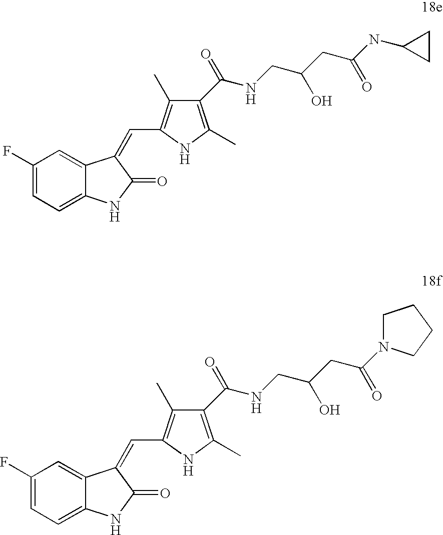

### EXAMPLE 19

Further amide examples of Example 5. The following examples 19a-d can be made by those skilled in the art following the above procedure and/or known procedures.

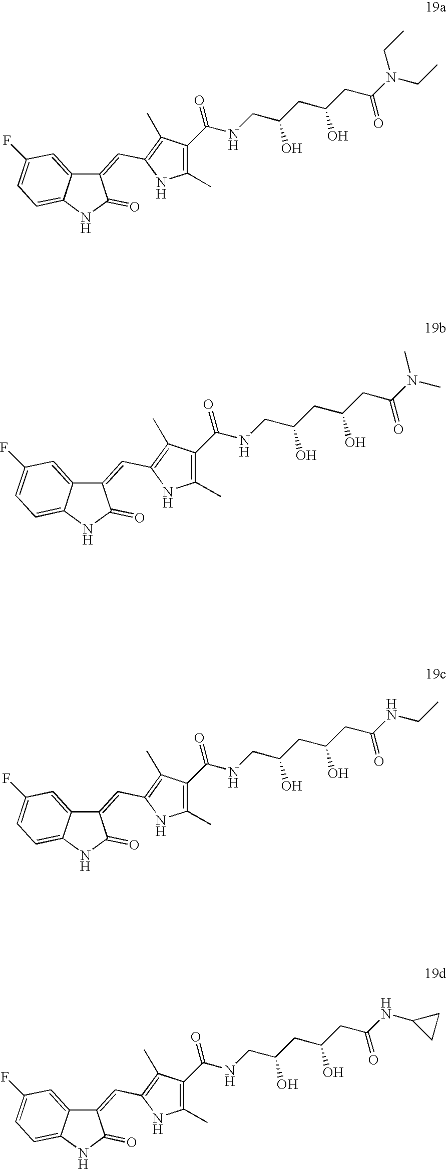

The compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

### EXAMPLE 20-269

Still further amide examples of Examples 1-5 are shown in the following table.

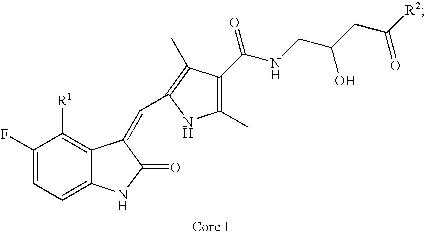

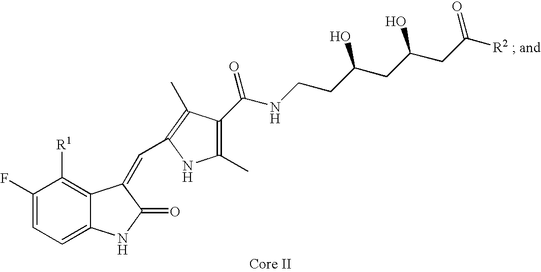

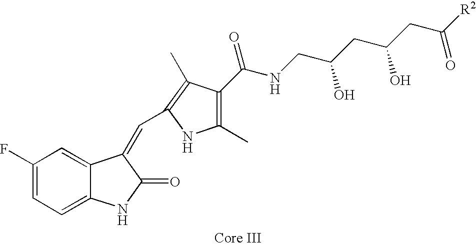

| Ex # | core | R1 | R2 |
|---|---|---|---|
| 20 | I | H | a |
| 21 | I | H | b |
| 22 | I | H | c |
| 23 | I | H | d |
| 24 | I | H | e |
| 25 | I | H | f |
| 26 | I | H | g |
| 27 | I | H | h |
| 28 | I | H | i |
| 29 | I | H | j |
| 30 | I | H | k |
| 31 | I | H | l |
| 32 | I | H | m |
| 33 | I | H | n |
| 34 | I | H | o |
| 35 | I | H | p |
| 36 | I | H | q |
| 37 | I | H | r |
| 38 | I | H | s |
| 39 | I | H | t |
| 40 | I | H | u |
| 41 | I | H | v |
| 42 | I | H | w |
| 43 | I | H | x |
| 44 | I | H | y |
| 45 | I | H | z |
| 46 | I | H | aa |
| 47 | I | H | ab |
| 48 | I | H | ac |
| 49 | I | H | ad |
| 50 | I | H | ae |
| 51 | I | H | af |
| 52 | I | H | ag |
| 53 | I | H | ah |
| 54 | I | H | ai |
| 55 | I | H | aj |
| 56 | I | H | ak |
| 57 | I | H | al |
| 58 | I | H | am |
| 59 | I | H | an |
| 60 | I | H | ao |
| 61 | I | H | ap |
| 62 | I | H | aq |
| 63 | I | H | ar |
| 64 | I | H | as |
| 65 | I | H | at |
| 66 | I | H | au |
| 67 | I | H | av |
| 68 | I | H | aw |
| 69 | I | H | ax |
| 70 | I | F | a |
| 71 | I | F | b |
| 72 | I | F | c |
| 73 | I | F | d |
| 74 | I | F | e |
| 75 | I | F | f |
| 76 | I | F | g |
| 77 | I | F | h |
| 78 | I | F | i |
| 79 | I | F | j |
| 80 | I | F | k |
| 81 | I | F | l |
| 82 | I | F | m |
| 83 | I | F | n |
| 84 | I | F | o |
| 85 | I | F | p |
| 86 | I | F | q |
| 87 | I | F | r |
| 88 | I | F | s |
| 89 | I | F | t |
| 90 | I | F | u |
| 91 | I | F | v |
| 92 | I | F | w |
| 93 | I | F | x |
| 94 | I | F | y |
| 95 | I | F | z |
| 96 | I | F | aa |
| 97 | I | F | ab |
| 98 | I | F | ac |
| 99 | I | F | ad |
| 100 | I | F | ae |
| 101 | I | F | af |
| 102 | I | F | ag |
| 103 | I | F | ah |
| 104 | I | F | ai |
| 105 | I | F | aj |
| 106 | I | F | ak |
| 107 | I | F | al |
| 108 | I | F | am |
| 109 | I | F | an |
| 110 | I | F | ao |
| 111 | I | F | ap |
| 112 | I | F | aq |
| 113 | I | F | ar |
| 114 | I | F | as |
| 115 | I | F | at |
| 116 | I | F | au |
| 117 | I | F | av |
| 118 | I | F | aw |
| 119 | I | F | ax |
| 120 | III | H | a |
| 121 | III | H | b |
| 122 | III | H | c |
| 123 | III | H | d |
| 124 | III | H | e |
| 125 | III | H | f |
| 126 | III | H | g |
| 127 | III | H | h |
| 128 | III | H | i |
| 129 | III | H | j |
| 130 | III | H | k |
| 131 | III | H | l |
| 132 | III | H | m |
| 133 | III | H | n |
| 134 | III | H | o |
| 135 | III | H | p |
| 136 | III | H | q |
| 137 | III | H | r |
| 138 | III | H | s |
| 139 | III | H | t |
| 140 | III | H | u |
| 141 | III | H | v |
| 142 | III | H | w |
| 143 | III | H | x |
| 144 | III | H | y |
| 145 | III | H | z |
| 146 | III | H | aa |
| 147 | III | H | ab |
| 148 | III | H | ac |
| 149 | III | H | ad |
| 150 | III | H | ae |
| 151 | III | H | af |
| 152 | III | H | ag |
| 153 | III | H | ah |
| 154 | III | H | ai |
| 155 | III | H | aj |
| 156 | III | H | ak |
| 157 | III | H | al |
| 158 | III | H | am |
| 159 | III | H | an |
| 160 | III | H | ao |
| 161 | III | H | ap |
| 162 | III | H | aq |
| 163 | III | H | ar |
| 164 | III | H | as |
| 165 | III | H | at |
| 166 | III | H | au |
| 167 | III | H | av |
| 168 | III | H | aw |
| 169 | III | H | ax |
| 170 | II | H | a |
| 171 | II | H | b |
| 172 | II | H | c |
| 173 | II | H | d |
| 174 | II | H | e |
| 175 | II | H | f |
| 176 | II | H | g |
| 177 | II | H | h |
| 178 | II | H | i |
| 179 | II | H | j |
| 180 | II | H | k |
| 181 | II | H | l |
| 182 | II | H | m |
| 183 | II | H | n |
| 184 | II | H | o |
| 185 | II | H | p |
| 186 | II | H | q |
| 187 | II | H | r |
| 188 | II | H | s |
| 189 | II | H | t |
| 190 | II | H | u |
| 191 | II | H | v |
| 192 | II | H | w |
| 193 | II | H | x |
| 194 | II | H | y |
| 195 | II | H | z |
| 196 | II | H | aa |
| 197 | II | H | ab |
| 198 | II | H | ac |
| 199 | II | H | ad |
| 200 | II | H | ae |
| 201 | II | H | af |
| 202 | II | H | ag |
| 203 | II | H | ah |
| 204 | II | H | ai |
| 205 | II | H | aj |
| 206 | II | H | ak |
| 207 | II | H | al |
| 208 | II | H | am |
| 209 | II | H | an |
| 210 | II | H | ao |
| 211 | II | H | ap |
| 212 | II | H | aq |
| 213 | II | H | ar |
| 214 | II | H | as |
| 215 | II | H | at |
| 216 | II | H | au |
| 217 | II | H | av |
| 218 | II | H | aw |
| 219 | II | H | ax |
| 220 | II | F | a |
| 221 | II | F | b |
| 222 | II | F | c |
| 223 | II | F | d |
| 224 | II | F | e |
| 225 | II | F | f |
| 226 | II | F | g |
| 227 | II | F | h |
| 228 | II | F | i |
| 229 | II | F | j |
| 230 | II | F | k |
| 231 | II | F | l |
| 232 | II | F | m |
| 233 | II | F | n |
| 234 | II | F | o |
| 235 | II | F | p |
| 236 | II | F | q |
| 237 | II | F | r |
| 238 | II | F | s |
| 239 | II | F | t |
| 240 | II | F | u |
| 241 | II | F | v |
| 242 | II | F | w |
| 243 | II | F | x |
| 244 | II | F | y |
| 245 | II | F | z |
| 246 | II | F | aa |
| 247 | II | F | ab |
| 248 | II | F | ac |
| 249 | II | F | ad |
| 250 | II | F | ae |
| 251 | II | F | af |
| 252 | II | F | ag |
| 253 | II | F | ah |
| 254 | II | F | ai |
| 255 | II | F | aj |
| 256 | II | F | ak |
| 257 | II | F | al |
| 258 | II | F | am |
| 259 | II | F | an |
| 260 | II | F | ao |
| 261 | II | F | ap |
| 262 | II | F | aq |
| 263 | II | F | ar |
| 264 | II | F | as |
| 265 | II | F | at |
| 266 | II | F | au |
| 267 | II | F | av |
| 268 | II | F | aw |
| 269 | II | F | ax |

In the above table, R² is selected from the following radicals: 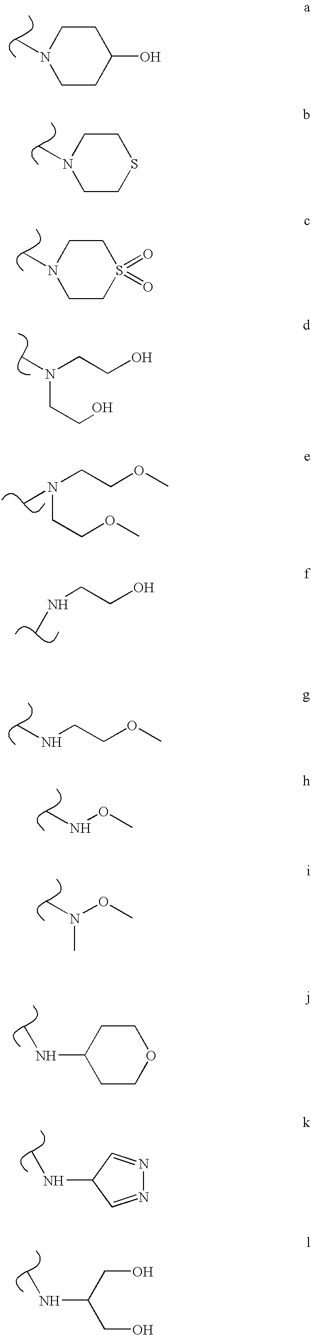 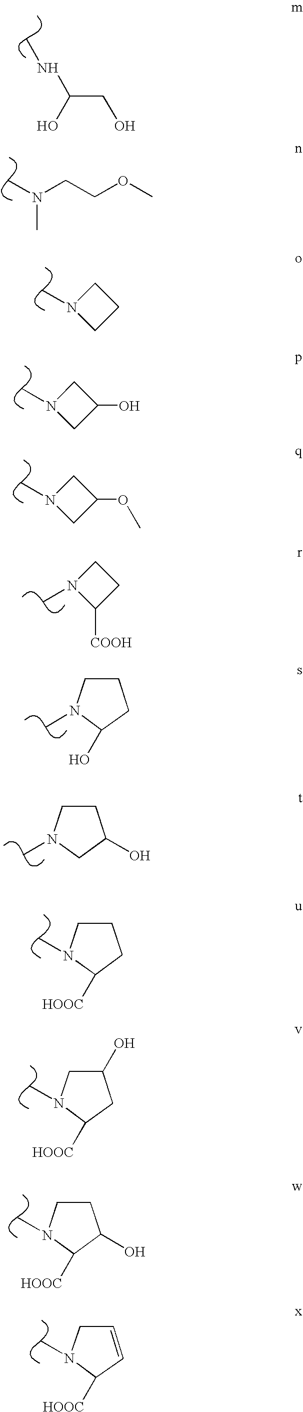 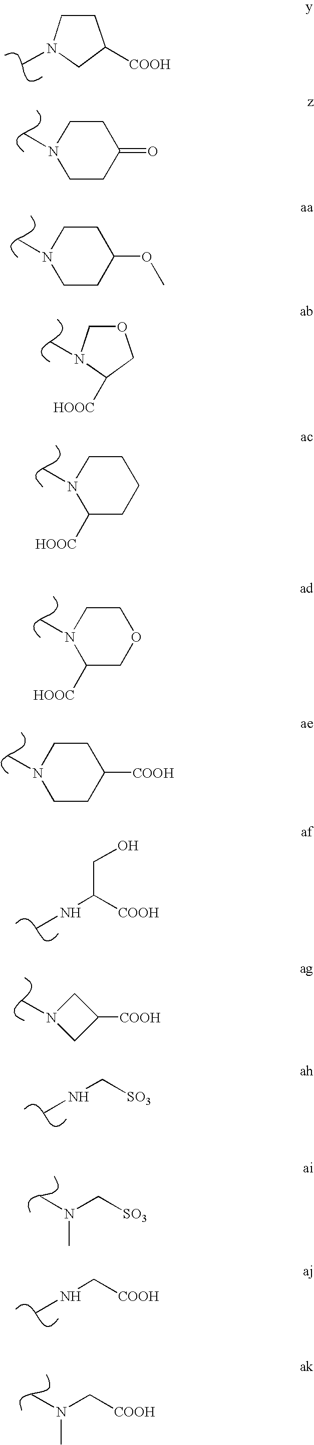 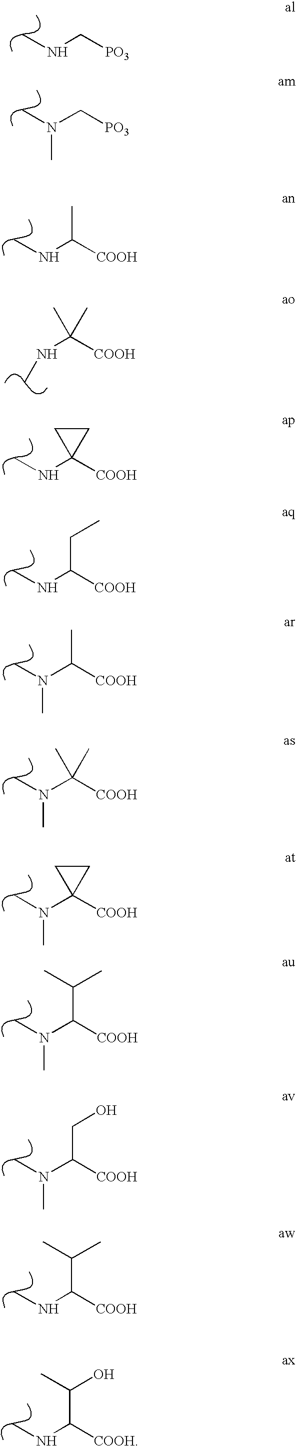
These amide examples 20-269 can be made by those skilled in the art following the above procedure and/or known procedures.

### EXAMPLES 301-307

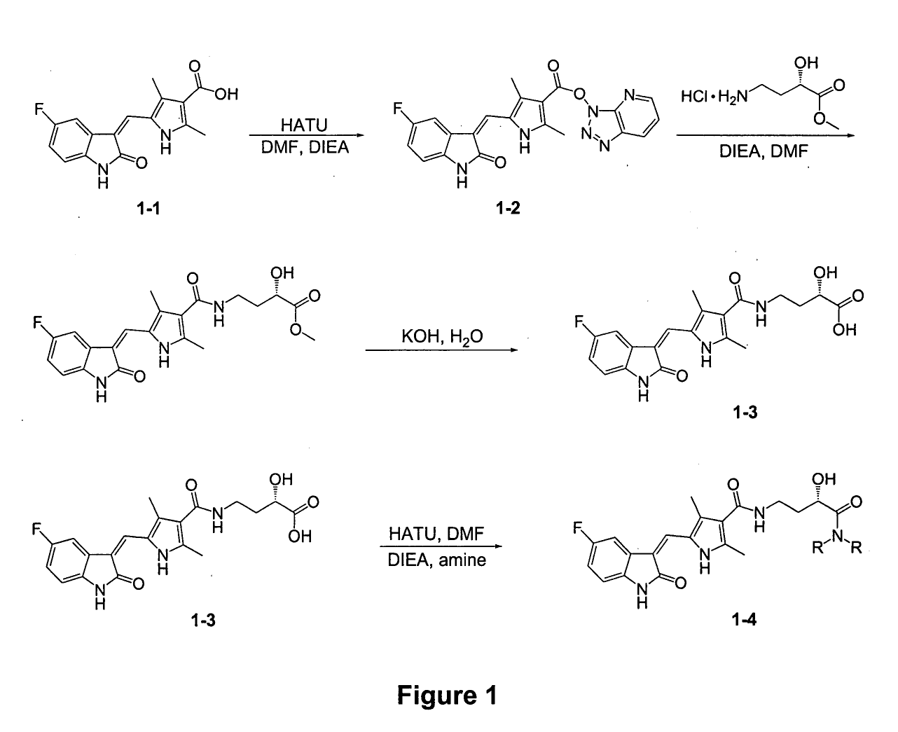

The synthesis of acid (1-3) and amides (1-4) is shown in FIG. 1.

### EXAMPLE 301

### (S)-4-({5-[5-Fluoro-2-oxo-1,2-dihydro-indol-(3Z)-ylidenemethyl]-2,4-dimethyl-1H-pyrrole-3-carbonyl}-amino)-2-hydroxy-butyric acid

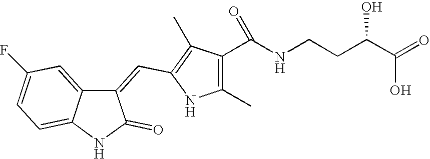 Compound 1-1 was prepared by following a literature procedure used for similar compounds (Li Sun, Chris Liang, et al; Discovery of 5-[5-Fluoro-2-oxo-1,2-dihydroindol-(3Z)-ylidenemethyl]-2,4-dimethyl-1H-pyrrole-3-carboxylic Acid (2-Diethylaminoethyl)amide, a Novel Tyrosine Kinase Inhibitor Targeting Vascular Endothelial and Platelet-Derived Growth Factor Receptor Tyrosine Kinase. J. Med. Chem. 2003, 46, 1116-1119). Compound 1-1 and DIEA (di-isopropyl ethylamine) (3 equiv) were suspended in dry DMF at room temperature (FIG. 1). After sonication (5 min), HATU (0.99 equiv) was added. The suspension became a clear solution after stirring approximately 1 minute at room temperature. Precipitation was observed after another 15 min. After DMF was removed under reduced pressure, anhydrous acetonitrile was added. The precipitate was collected by filtration, washed several times using acetonitrile, and dried under high vacuum for 2 days to give compound 1-2. LC-MS and NMR spectroscopy confirmed the structure of 1-2. To a solution of compound 1-2 (1.27 mmol) and DIEA (3 equiv) in DMF, the hydrogen chloride salt of methyl (2S)-4-amino-2-hydroxybutyrate (1.5 equiv, prepared earlier by refluxing the free amino acid (Aldrich) in dry methanol with 1.2 equiv HCl) was added. After stirring at 25° C. for 2 h (at which time LC-MS showed the completion of the reaction), KOH in water (5 equiv) was added, and stirring was continued until the hydrolysis was complete (monitored by LC-MS). The solvents were removed by evaporation under reduced pressure. Aqueous HCl (1N) was added to the residue, and the precipitate was collected by filtration, washed with water, and dried under high vacuum to obtain the title compound (0.5 g, 98%). LC-MS: single peak at 254 nm, MH⁺ calcd. for C₂₀H₂₀FN₃O₅: 402, obtained: 402.

### EXAMPLE 302-307

The general procedure for the synthesis of amides of Example 301: An amine (2 equiv) was added to a solution of the acid from Example 301, HATU (1.05 mmol), and DIEA (5 equiv) in DMF (5 mL). After the solution was stirred at 25° C. for 2 h, aqueous HCl (2 mL, 1N) was added. This solution was subjected to preparative HPLC to obtain the pure amide product, which was subsequently characterized by LC-MS and NMR spectroscopy.

### EXAMPLE 302

### 5-[5-Fluoro-2-oxo-1,2-dihydro-indol-(3Z)-ylidenemethyl]-2,4-dimethyl-1H-pyrrole-3-carboxylic acid ((S)-3-hydroxy-4-oxo-4-pyrrolidin-1-yl-butyl)-amide

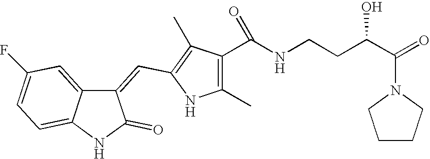 Preparative HPLC gave 32 mg of the title compound (34%) from 90 mg starting material (acid). LC-MS: single peak at 254 nm, MH⁺ calcd. for C₂₄H₂₇FN₄O₄: 455, obtained: 455.

### EXAMPLE 303

### 5-[5-Fluoro-2-oxo-1,2-dihydro-indol-(3Z)-ylidenemethyl]-2,4-dimethyl-1H-pyrrole-3-carboxylic acid [(S)-3-hydroxy-4-((R)-3-hydroxy-pyrrolidin-1-yl)-4-oxo-butyl]-amide

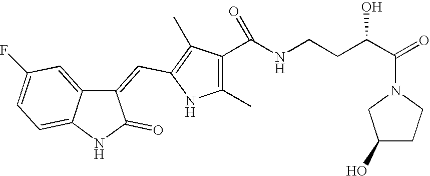 Preparative HPLC gave 27 mg of the title compound (41%) from 61 mg starting material (acid). LC-MS: single peak at 254 nm, MH⁺ calcd. for C₂₄H₂₇FN₄O₅: 471, obtained: 471.

### EXAMPLE 304

### 5-[5-Fluoro-2-oxo-1,2-dihydro-indol-(3Z)-ylidenemethyl]-2,4-dimethyl-1H-pyrrole-3-carboxylic acid ((S)-3-dimethylcarbamoyl-3-hydroxy-propyl)-amide

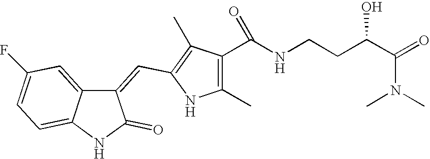 Preparative HPLC gave 22 mg of the title compound (37%) from 61 mg starting material (acid). LC-MS: single peak at 254 nm, MH⁺ calcd. for C₂₂H₂₅FN₄O₄: 429, obtained: 429.

### EXAMPLE 305

### 5-[5-Fluoro-2-oxo-1,2-dihydro-indol-(3Z)-ylidenemethyl]-2,4-dimethyl-1H-pyrrole-3-carboxylic acid ((S)-3-di-ethylcarbamoyl-3-hydroxy-propyl)-amide

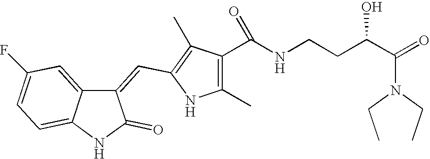 Preparative HPLC gave 43 mg of the title compound (27%) from 140 mg starting material (acid). LC-MS: single peak at 254 nm, MH⁺ calcd. for C₂₄H₂₉FN₄O₄: 457, obtained: 457.

### EXAMPLE 306

### 5-[5-Fluoro-2-oxo-1,2-dihydro-indol-(3Z)-ylidenemethyl]-2,4-dimethyl-1H-pyrrole-3-carboxylic acid ((S)-3-carbamoyl-3-hydroxy-propyl)-amide

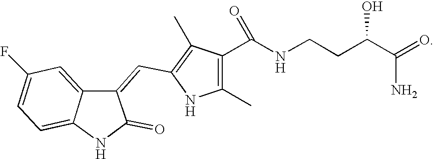 Preparative HPLC gave 15 mg of the title compound (20%) from 81 mg starting material (acid). LC-MS: single peak at 254 nm, MH⁺ calcd. for C₂₀H₂₁FN₄O₄: 401, obtained: 401.

### EXAMPLE 307

### 5-[5-Fluoro-2-oxo-1,2-dihydro-indol-(3Z)-ylidenemethyl]-2,4-dimethyl-1H-pyrrole-3-carboxylic acid ((S)-3-hydroxy-4-morpholin-4-yl-4-oxo-butyl)-amide

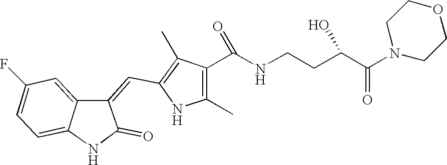 Preparative HPLC gave 18 mg of the title compound (21%) from 81 mg starting material (acid). LC-MS: single peak at 254 nm, MH⁺ calcd. for C₂₄H₂₇FN₄O₅: 471, obtained: 471.

### EXAMPLES 308-311

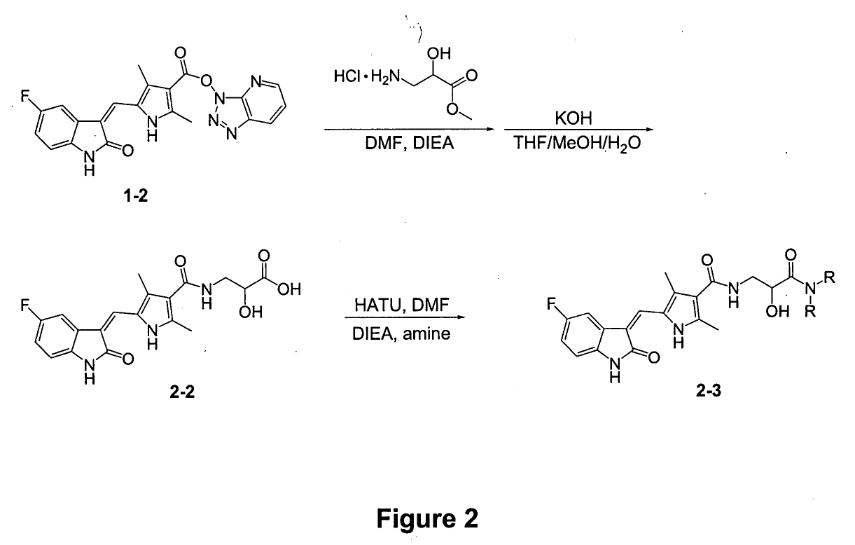

The synthesis of acid (2-2) and amides (2-3) is shown in FIG. 2.

### EXAMPLE 308

### 3-({5-[5-Fluoro-2-oxo-1,2-dihydro-indol-(3Z)-ylidenemethyl]-2,4-dimethyl-1H-pyrrole-3-carbonyl}-amino)-2-hydroxy-propionic acid

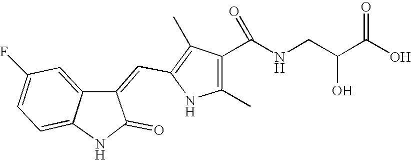 To a solution of compound 1-2 (1.0 mmol) and DIEA (3 equiv) in DMF, the HCl salt of methyl 3-amino-2-hydroxypropionate (1.2 equiv, prepared by refluxing the isoserine in dry methanol with 1.2 equiv HCl) was added. After stirring at 25° C. for 2 h (at which time LC-MS showed the completion of the reaction), KOH in water (5 equiv) was added, and the stirring was continued until the hydrolysis was complete (monitored by LC-MS). The solvents were removed by evaporation under reduced pressure. Aqueous HCl (1N) was added to the residue, and the precipitate was collected by filtration, washed with water, and dried under high vacuum to obtain compound 2-2 (0.33 g, 85%). LC-MS: single peak at 254 nm, MH⁺ calcd. for C₁₉H₁₈FN₃O₅: 388, obtained: 388.

### EXAMPLES 309-311

The general procedure for the synthesis of amides of Example 308: An amine (2 equiv) was added to a solution of the acid, HATU (1.05 mmol), and DIEA (5 equiv) in DMF (5 mL). After the solution was stirred at 25° C. for 2 h, aqueous HCl (2 mL, 1N) was added. This solution was subjected to preparative HPLC to obtain the pure amide product, which was subsequently characterized by LC-MS and NMR spectroscopy.

### EXAMPLE 309

### 5-[5-Fluoro-2-oxo-1,2-dihydro-indol-(3Z)-ylidenemethyl]-2,4-dimethyl-1H-pyrrole-3-carboxylic acid (2-dimethylcarbamoyl-2-hydroxy-ethyl)-amide

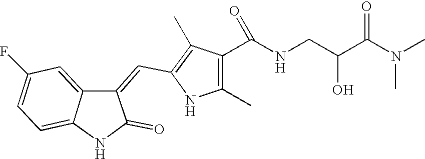 Preparative HPLC gave 50 mg of the title compound (72%) from 65 mg starting material (acid). LC-MS: single peak at 254 nm, MH⁺ calcd. for C₂₁H₂₃FN₄O₄: 415, obtained: 415. ¹H NMR (DMSO-d₆, 400 MHz) δ 13.67 (s, 1H), 10.87 (s, 1H), 7.75 (dd, J=2.4 Hz, 9.6 Hz, 1H), 7.70 (s, 1H), 7.56 (t, J=6.0 Hz, 1H), 6.92 (m, 1H), 6.83 (dd, J=4.8 Hz, 8.4 Hz, 1H), 4.53 (t, J=5.6 Hz, 1H), 3.48-3.25 (m, 2H), 3.08 (s, 3H), 2.85 (s, 3H), 2.43 (s, 3H), 2.41 (s, 3H).

### EXAMPLE 310

### 5-[5-Fluoro-2-oxo-1,2-dihydro-indol-(3Z)-ylidenemethyl]-2,4-dimethyl-1H-pyrrole-3-carboxylic acid (2-hydroxy-3-(morpholin-4-yl)-3-oxo-propyl)-amide

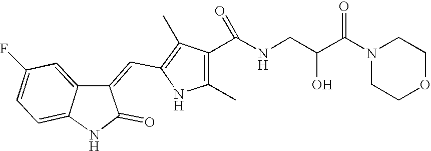 Preparative HPLC gave 14 mg of the title compound (18%) from 65 mg starting material (acid). LC-MS: single peak at 254 nm, MH⁺ calcd. for C₂₃H₂₅FN₄O₅: 457, obtained: 457. ¹H NMR (DMSO-d₆, 400 MHz) δ 13.68 (s, 1H), 10.90 (s, 1H), 7.75 (dd, J=2.4 Hz, 9.6 Hz, 1H), 7.71 (s, 1H), 7.60 (t, J=6.0 Hz, 1H), 6.92 (m, 1H), 6.83 (dd, J=4.4 Hz, 8.4 Hz, 1H), 5.2 (b, 1H), 4.51 (t, J=6.0 Hz, 1H), 3.65-3.35 (m, 10H), 2.43 (s, 3H), 2.41 (s, 3H).

### EXAMPLE 311

### 5-[5-Fluoro-2-oxo-1,2-dihydro-indol-(3Z)-ylidenemethyl]-2,4-dimethyl-1H-pyrrole-3-carboxylic acid [2-hydroxy-2-(methoxy-methyl-carbamoyl)-ethyl]-amide

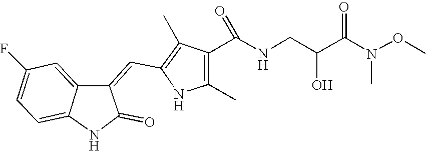 Preparative HPLC gave 16 mg of the title compound (18%) from 80 mg starting material (acid). LC-MS: single peak at 254 nm, MH⁺ calcd. for C₂₁H₂₃FN₄O₅: 431, obtained: 431. ¹H NMR (DMSO-d₆, 400 MHz) δ 13.67 (s, 1H), 10.89 (s, 1H), 7.75 (dd, J=2.0 Hz, 9.2 Hz, 1H), 7.70 (s, 1H), 7.55 (t, J=5.6 Hz, 1H), 6.92 (m, 1H), 6.82 (dd, J=4.8 Hz, 8.8 Hz, 1H), 4.51 (t, J=6.0 Hz, 1H), 3.74 (s, 3H), 3.55-3.40 (m, 2H), 3.13 (s, 3H), 2.42 (s, 3H), 2.41 (s, 3H).

### Exemplary Chiral Species

A general scheme for synthesizing chiral species of the invention is outline below: 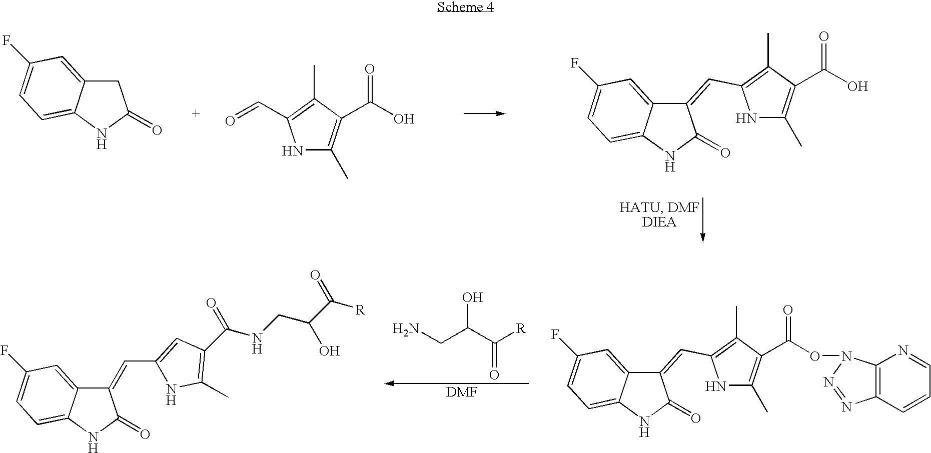
Step 1:

A mixture of 5-fluoro-1,3-dihydroindol-2-one (1.62 g, 10.2 mmol), 5-formyl-2,4-dimethyl-1H-pyrrole-3-carboxylic acid (1.96 g, 10.7 mmol), pyrrolidine (12 drops) and absolute ethanol was heated to reflux for 3 hours. The mixture was cooled to 25° C. and the solids were collected by filtration. The solids were stirred with ethanol (30 mL) at 72° C. for 30 min. The mixture was cooled to 25° C. and the solids were collected again by filtration, washed with ethanol (6 mL), and dried under vacuum overnight to give an orange solid (Z)-5-((5-fluoro-2-oxoindolin-3-ylidene)methyl)-2,4-dimethyl-1H-pyrrole-3-carboxylic acid (3.094 g, 96%). LC-ESIMS observed [M+H]⁺ 300.95 (calculated for C₁₆H₁₃FN₂O₃ 300.09).

Step 2:

(Z)-5-((5-fluoro-2-oxoindolin-3-ylidene)methyl)-2,4-dimethyl-1H-pyrrole-3-carboxylic acid (3.094 g, 10.3 mmol) was suspended in DMF (15 mL), and stirred for 5 minutes. DIEA (2.7 mL, 15.5 mmol) was then added and the mixture was stirred for 10 minutes. HATU (3.91 g, 10.28 mmol) was added and the reaction mixture was stirred at 25° C. for completion. LC/MS detected the completion of the reaction. Most of the DMF was removed and the residue was suspended in ACN and stirred for another 40 minutes. The solid was collected by filtration, washed with ACN, and dried under high vacuum overnight. (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (3.97 g, 92%) was obtained. LC-ESIMS observed [M+H]⁺ 418.68 (calculated for C₂₁H₁₅FN₆O₃ 418.12).

Step 3:

To (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (1.0 eq) DMF solution was added amine (1.2 eq), the reaction mixture was stirred at 25° C. for 2 h. LC/MS was applied to detect the completion of the reaction. Remove DMF under reduced pressure and the crude was precipitated with 5% diethylamine/methanol (3 mL) under sonication, the solid was collected by filtration and washed with 5% diethylamine/methanol (1 mL) twice.

### EXAMPLE 312

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid

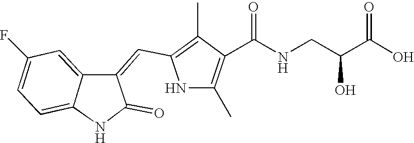

### Synthesis of (S)-methyl 3-amino-2-hydroxypropanoate hydrochloride

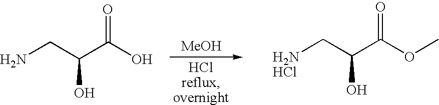 To the (S)-isoserine (921.6 mg, 8.77 mmol) in methanol (20 mL) was added concentrated HCl (0.5 mL), and the mixture was refluxed overnight. The mixture was cooled to 25° C. and the solvent was removed under reduced pressure. The crude material was dried and used directly in the next step.

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid methyl ester

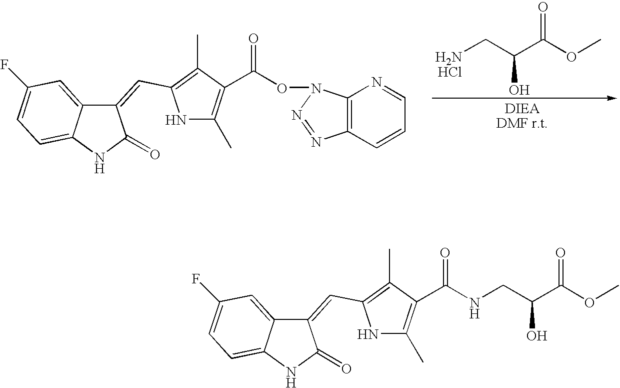 To (S)-methyl 3-amino-2-hydroxypropanoate hydrochloride (172.3 mg, 1.11 mmol) DMF solution was added DIEA (0.48 mL, 2.76 mmol) and the mixture was stirred at 25° C. for 20 minutes. (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (174.8 mg, 0.418 mmol) was added, and the mixture was stirred at 25° C. for the completion. The solvent was removed under reduced pressure to afford (S)-3-({5-[5-fluoro-2-oxo-1,2-dihydro-indol-(3Z)-ylidene-methyl]-2,4-dimethyl-1H-pyrrole-3-carbonyl}-amino)-2-hydroxypropanoic acid methyl ester (quantitative yield). The product was used in the next step with no purification. LC-ESIMS observed [M+H]⁺ 401.98 (calculated for C₂₀H₂₀FN₃O₅ 401.15).

### Synthesis of (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid

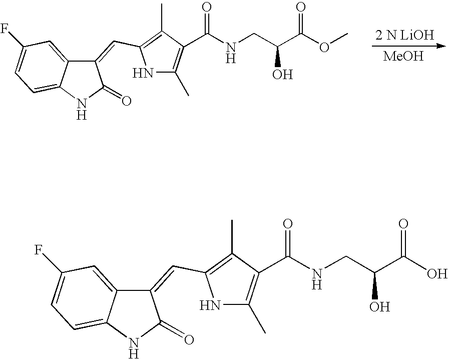 (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid methyl ester (167 mg, 0.418 mmol) and LiOH.H₂O (36 mg, 0.86 mmol) and methanol/water (10 ml/2 mL) was stirred at 25° C. overnight. Most of the solvent was removed under reduced pressure and excess 1N HCl was added to acidify the mixture. The orange solid was collected by filtration and washed with cold methanol to afford (S)-3-({5-[5-fluoro-2-oxo-1,2-dihydro-indol-(3Z)-ylidenemethyl]-2,4-dimethyl-1H-pyrrole-3-carbonyl}-amino)-2-hydroxypropanoic acid (yield 88%). LCESIMS observed [M+H]⁺ 387.96 (calculated for C₁₉H₁₈FN₃O₅ 387.12); ¹H NMR (400 MHz, DMSO-d₆) δ 13.91 (s, 1H), 10.89 (s, 1H), 7.75 (dd, J=9.6 Hz, 2.4 Hz, 1H), 7.70 (s, 1H), 7.57 (t, J=6.2 Hz, 1H), 6.92 (td, J=9.2 Hz, 2.4 Hz, 1H), 6.85-6.82 (m, 1H), 4.17-4.14 (m, 1H), 3.64 (s, 1H), 3.55-3.49 (m, 1H), 3.45-3.39 (m, 1H), 2.43 (s, 3H), 2.41 (s, 3H).

### EXAMPLES 313-317

The general procedure for the synthesis of amides: An amine (1.2 equiv) was added to a suspension of the (Z)-3H-[1,2,3]triazolo[4,5-b]pyridin-3-yl 5-((5-fluoro-2-oxoindolin-3-ylidene)methyl)-2,4-dimethyl-1H-pyrrole-3-carboxylate (1.0 eq) in DMF. The mixture was stirred at 25° C. for 2 h and LC/MS was applied to detect the completion of the reaction. The final solution was removed to get the crude solid, which was precipitated in 5% diethylamine/methanol, the solid was collected by filtration and washed with 5% diethylamine/methanol to afford the pure amide product, which was subsequently characterized by LC-MS and NMR spectroscopy.

### EXAMPLE 313

### Synthesis of 5-[5-Fluoro-2-oxo-1,2-dihydro-indol-(3Z)-ylidenemethyl]-2,4-dimethyl-1H-pyrrole-3-carboxylic acid ((S)-2-dimethylcarbamoyl-2-hydroxy-ethyl)-amide

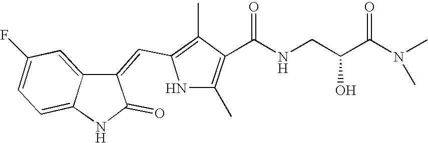

### Synthesis of (S)-3-(benzyloxycarbonyl)-2-hydroxypropanoic acid

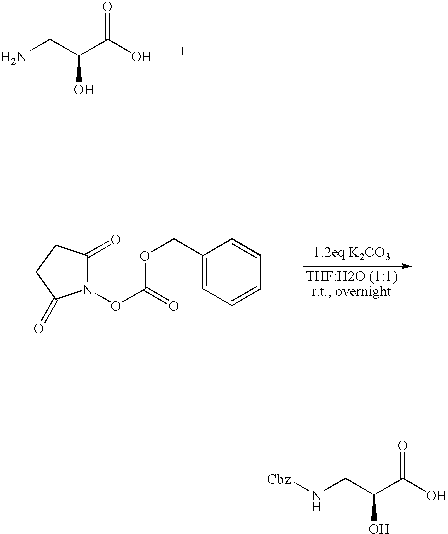 To the THF/water (50 mL/50 mL) solution of (S)-isoserine (2.429 g, 23.12 mmol) was added K₂CO₃ (3.834 g, 27.74 mmol) and N-(Benzyloxy-carbonyloxy)-succinimide (5.76 g, 23.11 mmol). The reaction mixture was stirred at 25° C. overnight. The reaction mixture was concentrated and diluted with EtOAc and acidified with excess HCl. The aqueous layer was extracted with EtOAc, and the combined organic layers were washed with dilute HCl, water, brine and dried over sodium sulfate. The solvent was removed under reduced pressure to afford (S)-3-(benzyloxycarbonyl)-2-hydroxypropanoic acid (5.11 g, 92%), which was used in the next step with no further purification. LC-ESIMS observed [M+H]⁺ 239.91 (calculated for C₁₁H₁₃NO₅ 239.08).

### Synthesis of (S)-benzyl 3-(dimethylamino)-2-hydroxy-3-oxopropylcarbamate

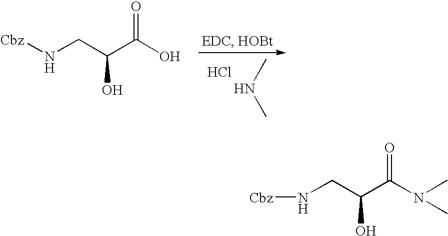 To (S)-3-(benzyloxycarbonyl)-2-hydroxypropanoic acid (377.8 mg, 1.58 mmol) in DMF (5 mL) was added dimethylamine hydrogen chloride (193.2 mg, 2.37 mmol) and DIEA (0.9 mL, 5.17 mmol). The mixture was then stirred for 5 min and EDC (454.3 mg, 2.37 mmol) and HOBt (320.3 mg, 2.37 mmol) were added. The reaction mixture was stirred at 25° C. overnight. DMF was removed under reduced pressure and the crude material was diluted with EtOAc and washed with saturated NaHCO₃. The aqueous layer was extracted twice with EtOAc and the combined organic layers were washed with water, 1N HCl and dried over NaSO₄. The solution was concentrated and the crude material was purified by flash chromatography with 0-20% MeOH/DCM to obtain the (S)-benzyl 3-(dimethylamino)-2-hydroxy-3-oxopropylcarbamate (349.2 mg, 83%). LC-ESIMS observed [M+H]⁺ 266.96 (calculated for C₁₃H₁₈N₂O₄ 266.13).

### Synthesis of (S)-3-amino-2-hydroxy-N,N-dimethylpropanamide

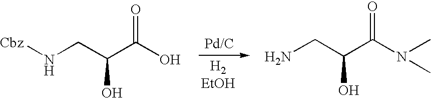 To the degassed (S)-benzyl 3-(dimethylamino)-2-hydroxy-3-oxopropylcarbamate (256.6 mg, 0.964 mmol) in ethanol (10 mL) was added Pd/C (10%, 30 mg) under argon protection, and then the mixture was degassed. The hydrogen balloon was used to provide the H₂ source. The reaction was stirred at 50° C. overnight. The mixture was filtered with Celite 521. The filtrate was evaporated to afford (S)-3-amino-2-hydroxy-N,N-dimethylpropanamide (125.2 mg, 98%). ¹H NMR (400 MHz, CDCl₃) δ 4.65 (t, J=5.4 Hz, 1H), 3.71-3.59 (m, 2H), 3.07 (s, 3H), 3.04 (s, 3H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((S)-2-dimethylcarbamoyl-2-hydroxy-ethyl)-amide

The title compound was obtained following the general procedure for amide synthesis (79%). LC-ESIMS observed [M+H]⁺ 414.97 (calculated for C₂₁H₂₃FN₄O₄ 414.17); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.04 (d, J=7.6 Hz, 1H), 4.53 (q, J=6.2 Hz, 1H), 3.47-3.41 (m, 1H), 3.36-3.30 (m, 1H), 3.08 (s, 3H), 2.85 (s, 3H), 2.43 (s, 3H), 2.40 (s, 3H).

### EXAMPLE 314

### Synthesis of 5-[5-Fluoro-2-oxo-1,2-dihydro-indol-(3Z)-ylidenemethyl]-2,4-dimethyl-1H-pyrrole-3-carboxylic acid ((S)-2-hydroxy-3-morpholin-4-yl-3-oxo-propyl)-amide

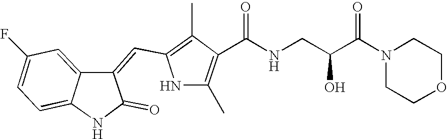

### Synthesis of (S)-benzyl 2-hydroxy-3-morpholino-3-oxopropylcarbamate

Similar method to synthesis of (S)-benzyl 3-(dimethylamino)-2-hydroxy-3-oxopropylcarbamate was applied and the title compound was obtained (yield 86%). LC-ESIMS observed [M+H]⁺ 408.96 (calculated for C₁₅H₂₀N₂O₅ 308.96).

### Synthesis of (S)-3-amino-2-hydroxy-1-morpholinopropan-1-one

Similar method to synthesis of (S)-3-amino-2-hydroxy-N,N-dimethylpropanamide was applied and the title compound was obtained (yield 94%). ¹H NMR (400 MHz, CDCl₃) δ 4.36-4.34 (m, 1H), 3.75-3.54 (m, 8H), 3.50 (d, J=4.0 Hz, 1H), 2.96-2.79 (m, 2H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((S)-2-hydroxy-3-morpholin-4-yl-3-oxo-propyl)-amide

The title compound was obtained following the general procedure for amide synthesis (75%). LC-ESIMS observed [M+H]⁺ 457.01 (calculated for C₂₃H₂₅FN₄O₅ 456.18); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.18 (d, J=8.8 Hz, 1H), 4.51 (q, J=6.0 Hz, 1H), 3.61-3.51 (m, 6H), 3.49-3.36 (m, 4H), 2.43 (s, 3H), 2.41 (s, 3H).

### EXAMPLE 315

### Synthesis of 5-[5-Fluoro-2-oxo-1,2-dihydro-indol-(3Z)-ylidenemethyl]-2,4-dimethyl-1H-pyrrole-3-carboxylic acid ((R)-2-dimethylcarbamoyl-2-hydroxy-ethyl)-amide

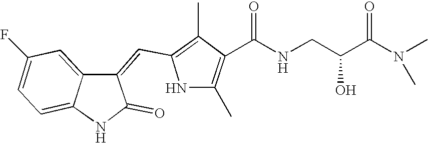

### Synthesis of (R)-methyl 3-azido-2-hydroxypropanoate

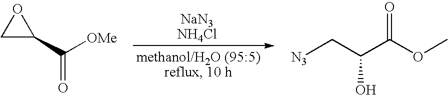 Sodium azide (5.487 g, 84.39 mmol) and ammonium chloride (2.257 g, 42.2 mmol) were added to a solution of methyl (2R)-glycidate (2.872 g, 28.13 mmol) in methanol (40 mL) and water (2 mL). After refluxing for 10 h, methanol was evaporated. The mixture was diluted in CHCl₃, washed with 1N HCl (5 mL) and extracted. After drying over sodium sulfate, the organic phase was concentrated and purified by flash chromatography to give the (R)-methyl 3-azido-2-hydroxypropanoate (2.82 g, 69%). ¹H NMR (400 MHz, CDCl₃) δ 4.39-4.36 (m, 1H), 3.84 (s, 3H), 3.67-3.48 (m, 2H), 3.18 (d, J=4.0 Hz, 1H).

### Synthesis of (R)-3-azido-2-hydroxypropanoic acid

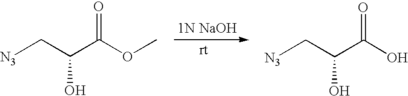 To a solution of (R)-methyl 3-azido-2-hydroxypropanoate (7.3 g, 50.3 mmol) in MeOH (150 mL) at 0° C. was added 1N NaOH (65 mL, 65 mmol). After being stirred at room temperature for 1 h, the mixture was acidified by 1N HCl and extracted with EtOAc. The organic layers were dried over sodium sulfate and concentrated in vacuo to give the acid as a white solid. The compound was used in the next step with no further purification.

### Synthesis of (R)-3-azido-2-hydroxy-N,N-dimethylpropanamide

Similar method to synthesis of (S)-benzyl 3-(dimethylamino)-2-hydroxy-3-oxopropylcarbamate was applied and the title compound was obtained (yield 93%). ¹H NMR (400 MHz, CDCl₃) δ 4.39-4.36 (m, 1H), 3.67-3.48 (m, 2H), 3.18 (d, J=4.0 Hz, 1H), 3.08 (s, 3H), 3.04 (s, 3H).

### Synthesis of (R)-3-amino-2-hydroxy-N,N-dimethylpropanamide

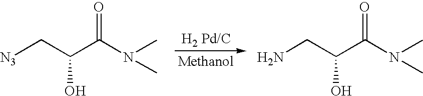

To the degassed (R)-3-azido-2-hydroxy-N,N-dimethylpropanamide (8.37 g, 46.6 mmol) in ethanol (150 mL) was added Pd/C (10%, 837 mg) under argon protection, and then the mixture was degassed. A hydrogen balloon was used to provide an H₂ source. The reaction was stirred at 25° C. for 2 h, and TLC was applied to detect the completion of the reaction. The mixture was filtered with Celite 521. The filtrate was evaporated to afford the desired compound (5.38 g, 87%). ¹H NMR (400 MHz, CDCl₃) δ 4.65 (t, J=5.4 Hz, 1H), 3.71-3.59 (m, 2H), 3.07 (s, 3H), 3.04 (s, 3H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-dimethylcarbamoyl-2-hydroxy-ethyl)-amide

The title compound was obtained following the general procedure for amide synthesis (yield 85%), LC-ESIMS observed [M+H]⁺ 414.97 (calculated for C₂₁H₂₃FN₄O₄ 414.17); ¹H NMR (400 MHz, DMSO-d₆) δ 13.67 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.04 (d, J=7.6 Hz, 1H), 4.53 (q, J=6.2 Hz, 1H), 3.47-3.41 (m, 1H), 3.36-3.30 (m, 1H), 3.08 (s, 3H), 2.85 (s, 3H), 2.43 (s, 3H), 2.40 (s, 3H).

### EXAMPLE 316

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-3-morpholin-4-yl-3-oxo-propyl)-amide

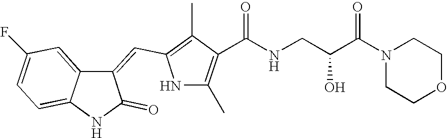

### Synthesis of (R)-3-azido-2-hydroxy-1-morpholinopropan-1-one

Similar method to synthesis of (S)-benzyl 3-(dimethylamino)-2-hydroxy-3-oxopropylcarbamate was applied and the title compound was obtained (yield 90%), ¹H NMR (400 MHz, CDCl₃) δ 4.55 (t, J=5.2 Hz, 1H), 3.71-3.60 (m, 6H), 3.48-3.41 (m, 3H), 3.40-3.35 (m, 2H).

### Synthesis of (R)-3-amino-2-hydroxy-1-morpholinopropan-1-one

A similar method to synthesis of (R)-3-amino-2-hydroxy-N,N-dimethylpropanamide was used and the title compound was obtained in high yield (yield 95%). ¹H NMR (400 MHz, CDCl₃) δ 4.36-4.34 (m, 1H), 3.75-3.54 (m, 8H), 3.50 (d, J=4.0 Hz, 1H), 2.96-2.79 (m, 2H), 1.94 (broad s, 2H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-3-morpholin-4-yl-3-oxo-propyl)-amide

The title compound was obtained following the general procedure for amide synthesis (yield 75%). LC-ESIMS observed [M+H]⁺ 457.01 (calculated for C₂₃H₂₅FN₄O₅ 456.18); ¹H NMR (400 MHz, DMSO-d₆) δ 13.68 (s, 1H), 10.89 (s, 1H), 7.76 (dd, J=9.6 Hz, 2.4 Hz, 1H), 7.71 (s, 1H), 7.59 (t, J=6.2 Hz, 1H), 6.92 (td, J=9.2 Hz, 2.4 Hz, 1H), 6.85-6.82 (m, 1H), 5.18 (d, J=6.4 Hz, 1H), 4.54-4.49 (m, 1H), 3.61-3.51 (m, 6H), 3.49-3.36 (m, 4H), 2.43 (s, 3H), 2.41 (s, 3H).

### EXAMPLE 317

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-2-methylcarbamoyl-ethyl)-amide

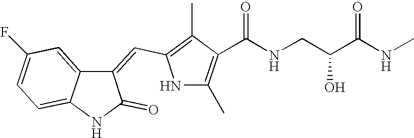

### Synthesis of (R)-3-azido-2-hydroxy-N-methylpropanamide

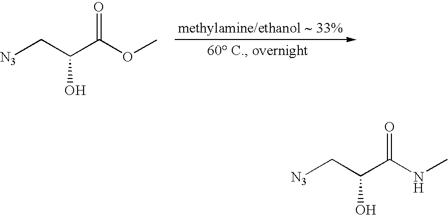 (R)-methyl 3-azido-2-hydroxypropanoate (505.4 mg, 3.48 mmol) and methylamine ethanol solution (15 mL) was sealed and stirred at 60° C. oil bath overnight. TLC analysis was applied to detect the reaction completion. The solvent was removed and the crude was purified by flash chromatography (0-20% Methanol/DCM) to afford (R)-3-azido-2-hydroxy-N-methylpropan-amide (385.2 mg, yield 77%), ¹H NMR (400 MHz, CDCl₃) δ 6.90-6.70 (broad s, 1H), 4.28-4.24 (m, 1H), 3.69-3.57 (m, 3H), 2.87 (d, J=5.6 Hz, 3H).

### Synthesis of (R)-3-amino-2-hydroxy-N-methylpropanamide

Similar method to synthesis of (R)-3-amino-2-hydroxy-N,N-dimethylpropan-amide was used and the title compound was obtained (yield 98%). ¹H NMR (400 MHz, CDCl₃) δ 7.05 (broad s, 1H), 3.97 (t, J=5.6 Hz, 1H), 3.12-2.96 (m, 2H), 2.85 (d, J=5.2 Hz, 3H), 1.90 (broad, 2H).

### Synthesis of 5-[5-Fluoro-2-oxo-1,2-dihydro-indol-(3Z)-ylidenemethyl]-2,4-dimethyl-1H-pyrrole-3-carboxylic acid ((R)-2-hydroxy-2-methylcarbamoyl-ethyl)-amide

The title compound was obtained following the general procedure for amide synthesis (yield 86%), LC-ESIMS observed [M+H]⁺ 400.96 (calculated for C₂₀H₂₁FN₄O₄ 400.15); ¹H NMR (400 MHz, DMSO-d₆) δ 13.69 (s, 1H), 10.89 (s, 1H), 7.87 (d, J=4.8 Hz, 1H), 7.76 (dd, J=9.6 Hz, 2.4 Hz, 1H), 7.71 (s, 1H), 7.52 (t, J=5.6 Hz, 1H), 6.95-6.90 (m, 1H), 6.85-6.82 (m, 1H), 5.83 (d, J=5.2 Hz, 1H), 4.07-4.03 (m, 1H), 3.57-3.51 (m, 1H), 3.37-3.30 (m, 1H), 2.62 (d, J=4.4 Hz, 3H) 2.45 (s, 3H), 2.42 (s, 3H).

The compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

### EXAMPLES 316-515

Still further amide examples are shown in the following table:

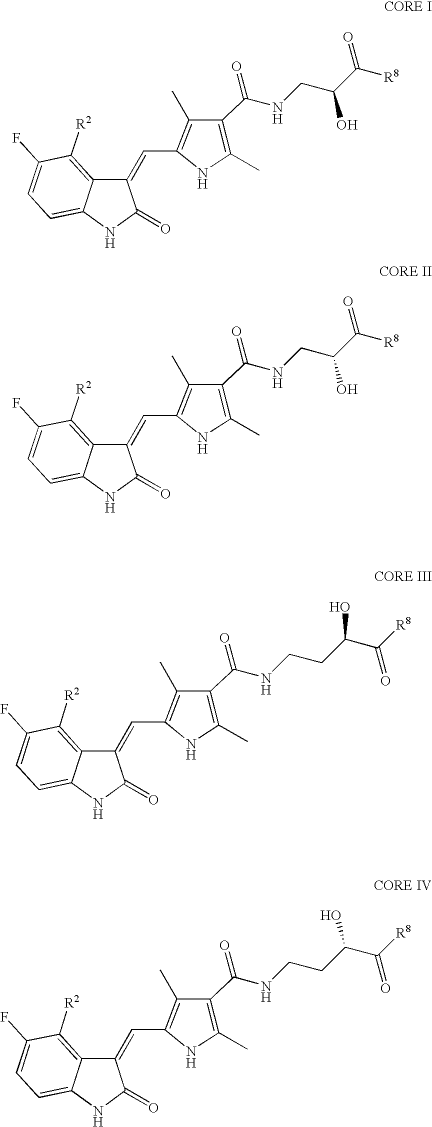

In the above core structures, R² is selected from the group consisting of hydrogen and fluoro; and R⁸ is selected from the group consisting of hydroxyl or radicals represented by the following structures:

| Ex# | Core | R⁸ |
|---|---|---|
| 316 | I | a |
| 317 | I | b |
| 318 | I | c |
| 319 | I | d |
| 320 | I | e |
| 321 | I | f |
| 322 | I | g |
| 323 | I | h |
| 324 | I | i |
| 325 | I | j |
| 326 | I | k |
| 327 | I | l |
| 328 | I | m |
| 329 | I | n |
| 330 | I | o |
| 331 | I | p |
| 332 | I | q |
| 333 | I | r |
| 334 | I | s |
| 335 | I | t |
| 336 | I | u |
| 337 | I | v |
| 338 | I | w |
| 339 | I | x |
| 340 | I | y |
| 341 | I | z |
| 342 | I | aa |
| 343 | I | ab |
| 344 | I | ac |
| 345 | I | ad |
| 346 | I | ae |
| 347 | I | af |
| 348 | I | ag |
| 349 | I | ah |
| 350 | I | ai |
| 351 | I | aj |
| 352 | I | ak |
| 353 | I | al |
| 354 | I | am |
| 355 | I | an |
| 356 | I | ao |
| 357 | I | ap |
| 358 | I | aq |
| 359 | I | ar |
| 360 | I | as |
| 361 | I | at |
| 362 | I | au |
| 363 | I | av |
| 364 | I | aw |
| 365 | I | ax |
| 366 | II | a |
| 367 | II | b |
| 368 | II | c |
| 369 | II | d |
| 370 | II | e |
| 371 | II | f |
| 372 | II | g |
| 373 | II | h |
| 374 | II | i |
| 375 | II | j |
| 376 | II | k |
| 377 | II | l |
| 378 | II | m |
| 379 | II | n |
| 380 | II | o |
| 381 | II | p |
| 382 | II | q |
| 383 | II | r |
| 384 | II | s |
| 385 | II | t |
| 386 | II | u |
| 387 | II | v |
| 388 | II | w |
| 389 | II | x |
| 390 | II | y |
| 391 | II | z |
| 392 | II | aa |
| 393 | II | ab |
| 394 | II | ac |
| 395 | II | ad |
| 396 | II | ae |
| 397 | II | af |
| 398 | II | ag |
| 399 | II | ah |
| 400 | II | ai |
| 401 | II | aj |
| 402 | II | ak |
| 403 | II | al |
| 404 | II | am |
| 405 | II | an |
| 406 | II | ao |
| 407 | II | ap |
| 408 | II | aq |
| 409 | II | ar |
| 410 | II | as |
| 411 | II | at |
| 412 | II | au |
| 413 | II | av |
| 414 | II | aw |
| 415 | II | ax |
| 416 | III | a |
| 417 | III | b |
| 418 | III | c |
| 419 | III | d |
| 420 | III | e |
| 421 | III | f |
| 422 | III | g |
| 423 | III | h |
| 424 | III | i |
| 425 | III | j |
| 426 | III | k |
| 427 | III | l |
| 428 | III | m |
| 429 | III | n |
| 430 | III | o |
| 431 | III | p |
| 432 | III | q |
| 433 | III | r |
| 434 | III | s |
| 435 | III | t |
| 436 | III | u |
| 437 | III | v |
| 438 | III | w |
| 439 | III | x |
| 440 | III | y |
| 441 | III | z |
| 442 | III | aa |
| 443 | III | ab |
| 444 | III | ac |
| 445 | III | ad |
| 446 | III | ae |
| 447 | III | af |
| 448 | III | ag |
| 449 | III | ah |
| 450 | III | ai |
| 451 | III | aj |
| 452 | III | ak |
| 453 | III | al |
| 454 | III | am |
| 455 | III | an |
| 456 | III | ao |
| 457 | III | ap |
| 458 | III | aq |
| 459 | III | ar |
| 460 | III | as |
| 461 | III | at |
| 462 | III | au |
| 463 | III | av |
| 464 | III | aw |
| 465 | III | ax |
| 466 | IV | a |
| 467 | IV | b |
| 468 | IV | c |
| 469 | IV | d |
| 470 | IV | e |
| 471 | IV | f |
| 472 | IV | g |
| 473 | IV | h |
| 474 | IV | i |
| 475 | IV | j |
| 476 | IV | k |
| 477 | IV | l |
| 478 | IV | m |
| 479 | IV | n |
| 480 | IV | o |
| 481 | IV | p |
| 482 | IV | q |
| 483 | IV | r |
| 484 | IV | s |
| 485 | IV | t |
| 486 | IV | u |
| 487 | IV | v |
| 488 | IV | w |
| 489 | IV | x |
| 490 | IV | y |
| 491 | IV | z |
| 492 | IV | aa |
| 493 | IV | ab |
| 494 | IV | ac |
| 495 | IV | ad |
| 496 | IV | ae |
| 497 | IV | af |
| 498 | IV | ag |
| 499 | IV | ah |
| 500 | IV | al |
| 501 | IV | aj |
| 502 | IV | ak |
| 503 | IV | al |
| 504 | IV | am |
| 505 | IV | an |
| 506 | IV | ao |
| 507 | IV | ap |
| 508 | IV | aq |
| 509 | IV | ar |
| 510 | IV | as |
| 511 | IV | at |
| 512 | IV | au |
| 513 | IV | av |
| 514 | IV | aw |
| 515 | IV | ax |

In the above table, R⁸ is selected from the following radicals: 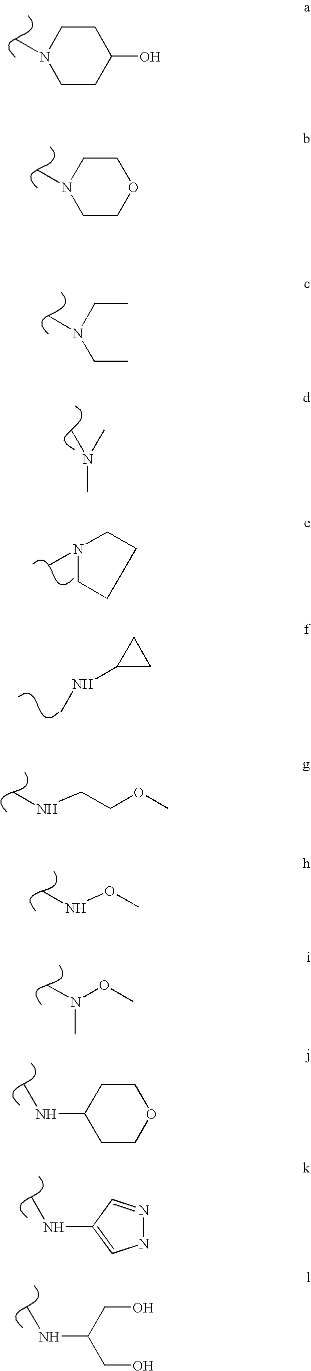 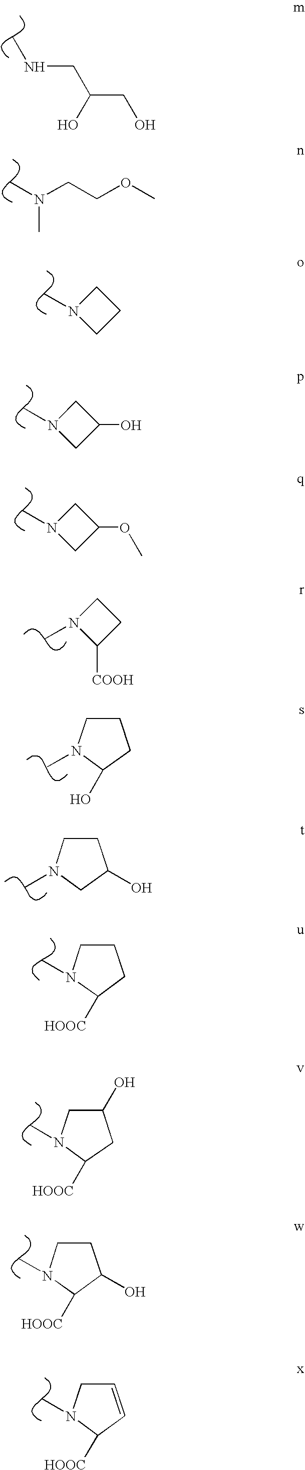 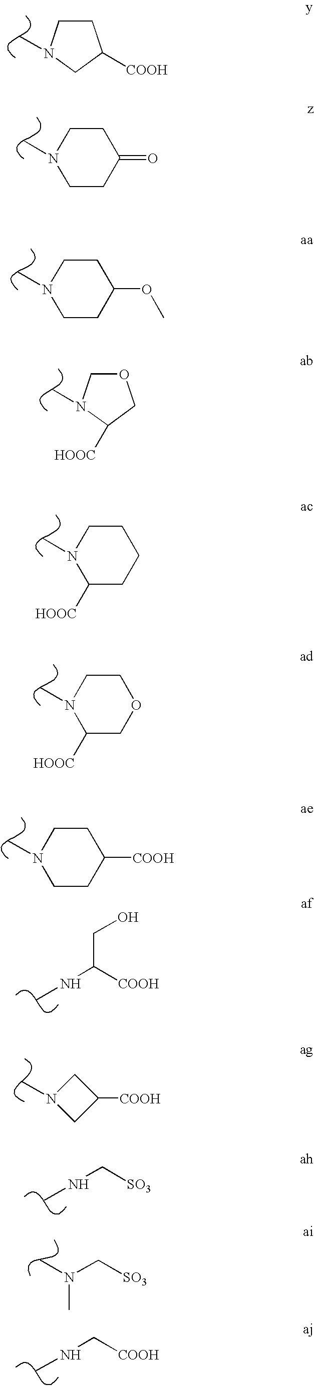 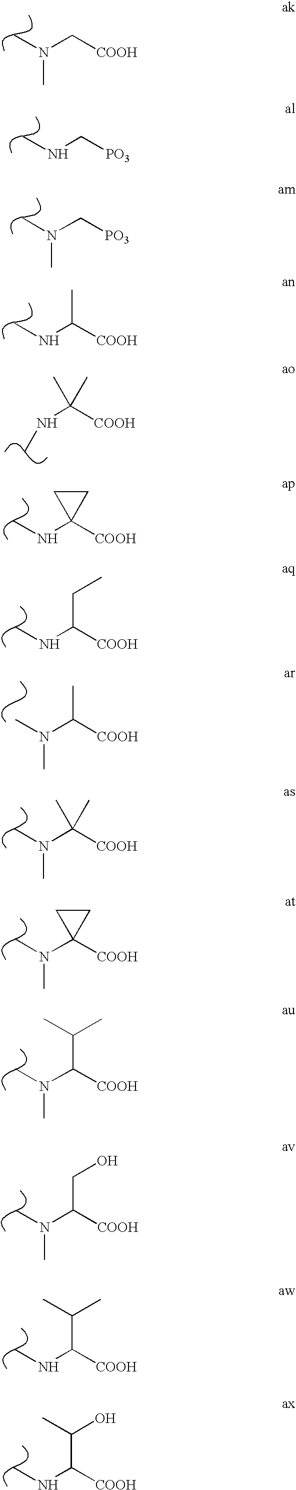
These amide examples 320-515 can be made by those skilled in the art following the above procedure and/or known procedures.
VEGFR Biochemical Assay
The compounds were assayed for biochemical activity by Upstate Ltd at Dundee, United Kingdom, according to the following procedure. In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [γ-³³P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.
Cellular Assay: HUVEC: VEGF Induced Proliferation
The compounds were assayed for cellular activity in the VEGF induced proliferation of HUVEC cells. HUVEC cells (Cambrex, CC-2517) were maintained in EGM (Cambrex, CC-3124) at 37° C. and 5% CO₂. HUVEC cells were plated at a density 5000 cells/well (96 well plate) in EGM. Following cell attachment (1 hour) the EGM-medium was replaced by EBM (Cambrex, CC-3129)+0.1% FBS (ATTC, 30-2020) and the cells were incubated for 20 hours at 37° C. The medium was replaced by EBM+1% FBS, the compounds were serial diluted in DMSO and added to the cells to a final concentration of 0-5,000 nM and 1% DMSO. Following a 1 hour pre-incubation at 37° C. cells were stimulated with 10 ng/ml VEGF (Sigma, V7259) and incubated for 45 hours at 37° C. Cell proliferation was measured by BrdU DNA incorporation for 4 hours and BrdU label was quantitated by ELISA (Roche kit, 16472229) using 1M H₂SO₄ to stop the reaction. Absorbance was measured at 450 nm using a reference wavelength at 690 nm.

### DETAILED DESCRIPTION OF FIGURES

FIG. 1 is a scheme showing the synthesis of the acid 1-3 and the corresponding amides, 1-4. The starting carboxylic acid is synthesized according to the supplemental material of Sun, L.; et al., J. Med. Chem. 2003, 46, 1116-1119. The intermediate, 1-2, is formed by reaction of the acid with HATU in the presence of 3 equivalents of Hunig's base, or di-isopropyl ethylamine (DIEA). A solid precipitated after 15 minutes and the solid was isolated and characterized. This was then reacted with 1.5 equivalents of methyl (2S)-4-amino-2-hydroxybutyrate in DMF and 3 equivalents of Hunig's base. The methyl ester was hydrolyzed with 5 equivalents of KOH in water. Acidifying the reaction mixture enabled the isolation of the free acid, 1-3. This acid was then reacted with HATU in the presence of 3 equivalents of DIEA in DMF. An amine (2 equivalents) was added and after reacting for 2 hours, the amide was isolated by preparative HPLC.

FIG. 2 is a scheme showing the synthesis of the amide series, 2-3. The activated acid, 1-2 is reacted with methyl 3-amino-2-hydroxypropionate hydrochloride in the presence of 3 equivalents of base (DIEA) in DMF. After stirring for 2 h at room temperature, KOH, 5 equivalents, in water was added and stirring continued until ester hydrolysis was complete. The acid was isolated after acidification of the reaction mixture. The free acid was then added to HATU (1.05 equivalent), DIEA (5 equivalents), and an amine (2 equivalents) in DMF. The mixture was stirred for 2 h at room temperature and the mixture was acidified. The pure product was isolated by preparative HPLC.

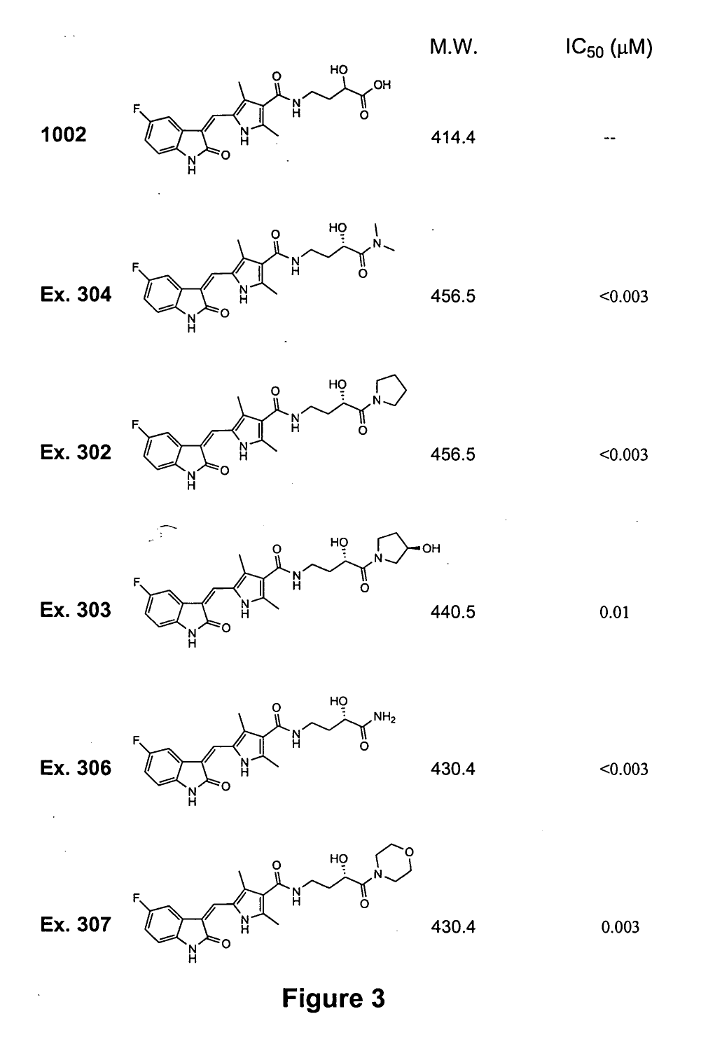

FIG. 3 shows example compounds and some of their activities against KDR. The units of IC₅₀ is in μM.

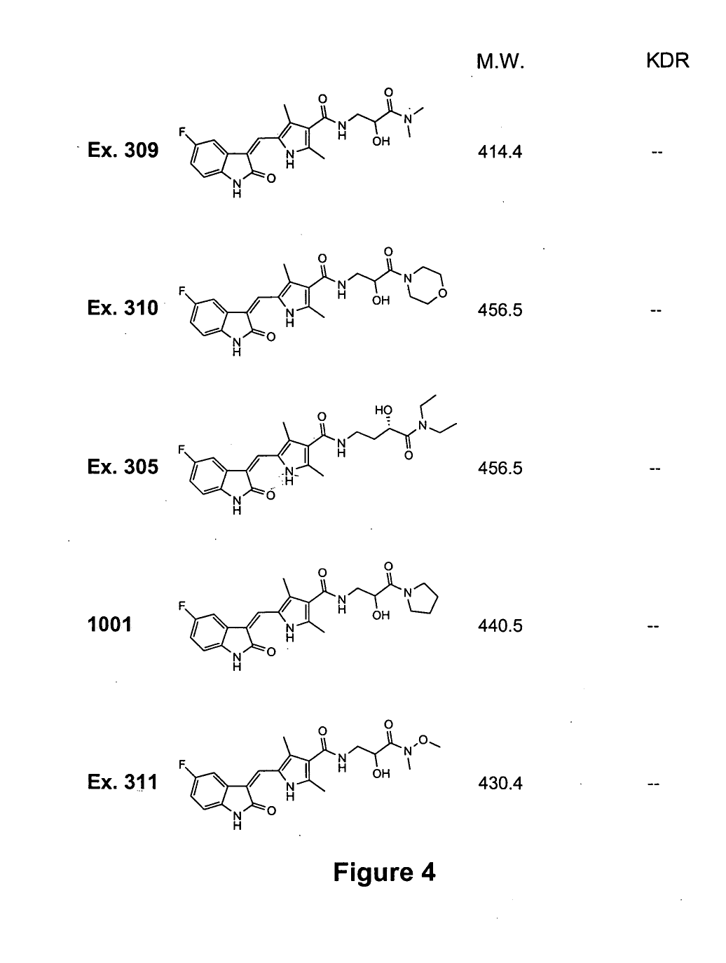

FIG. 4 shows additional compounds that were tested for activity.

What is claimed is:

1. A compound represented by Formula (I): 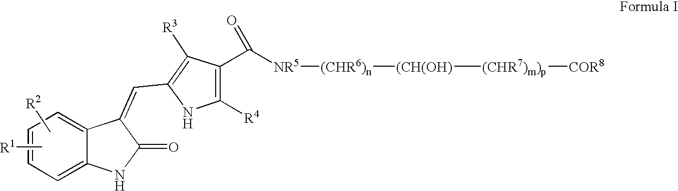
wherein:
R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl;
R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino;
R³ is selected from the group consisting of hydrogen, (C1-C6) alkyl, (C6-C10) aryl, (C5-C10) heteroaryl, and amide;
R⁴, R⁵ and R⁶ are independently selected from the group consisting of hydrogen and (C1-C6) alkyl;
each R⁷ is independently selected from the group consisting of hydrogen, (C1-C6) alkyl and hydroxyl;
R⁸ is selected from the group consisting of hydroxy, (C1-C6) O-alkyl, (C3-C8) O-cycloalkyl, and NR⁹R¹⁰; where R⁹ and R¹⁰ are independently selected from the group consisting of hydrogen, (C1-C6) alkyl, (C1-C6) hydroalkyl, (C1-C6) dihydroxyalkyl, (C1-C6) alkoxy, (C1-C6) alkyl carboxylic acid, (C1-C6) alkyl phosphoric acid, (C1-C6) alkyl sulfuric acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁹ and R¹⁰ together with N forms a (C5-C8) heterocyclic ring either unsubstituted or substituted with one or more hydroxyls, ketones, ethers, and carboxylic acids; and
n and m are independently 0, 1, 2, or 3; p is 1, 2, or 3;
or, a pharmaceutically acceptable salt, its tautomer, a pharmaceutically acceptable salt of its tautomer, or a prodrug thereof.

2. The compound, salt, tautomer, or prodrug according to claim 1 selected from the group represented by the following structures: 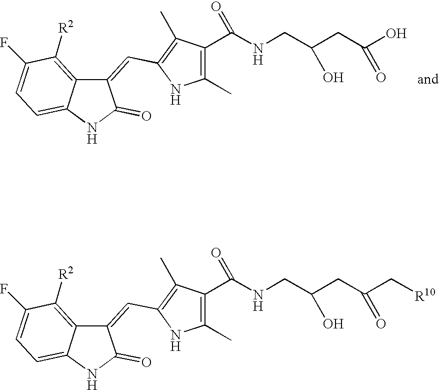
wherein R² is selected from the group consisting of hydrogen and fluoro.

3. The compound, salt, tautomer, or prodrug according to claim 1 represented by the following structure:

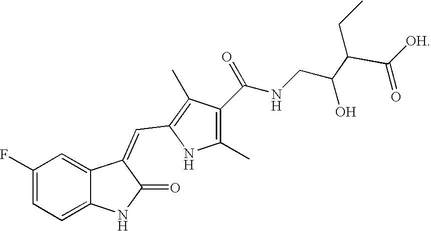

4. The compound, salt, tautomer, or prodrug according to claim 1 represented by Formula (II): 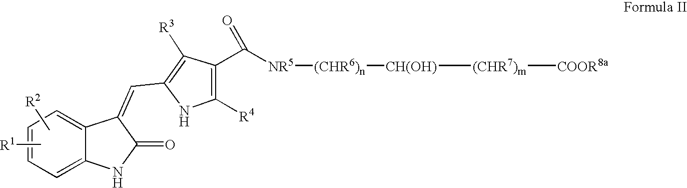
wherein R^{8a} is selected from the group consisting of hydrogen, (C1-C6) alkyl, and (C3-C8) cycloalkyl.

5. The compound, salt, tautomer, or prodrug according to claim 4, wherein:
R¹ and R² are independently selected from the group consisting of hydrogen and fluoro;
R³ and R⁴ are methyl;
R⁵, R⁶, R⁷ and R^{8a} are hydrogen; and
n and m are independently 0, 1, or 2.

6. The compound, salt, tautomer, or prodrug according to claim 5 selected from the group consisting of:

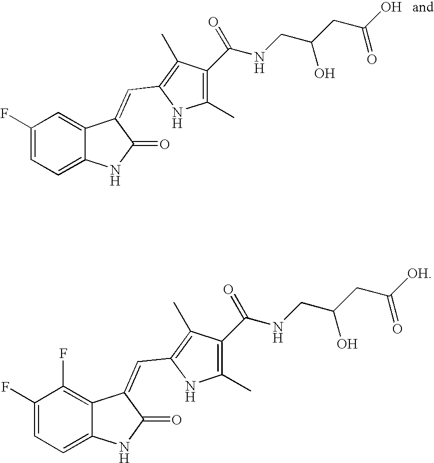

7. The compound, salt, tautomer, or prodrug according to claim 5 represented by the following structure:

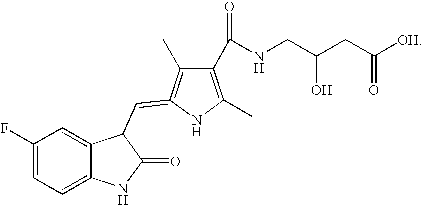

8. The compound, salt, tautomer, or prodrug according to claim 5 represented by the following structure:

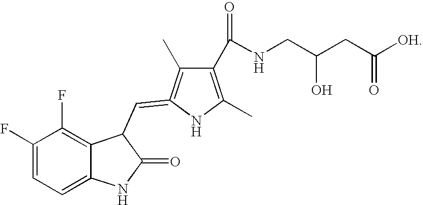

9. A compound, salt, tautomer, or prodrug according to claim 1 represented by Formula (III): 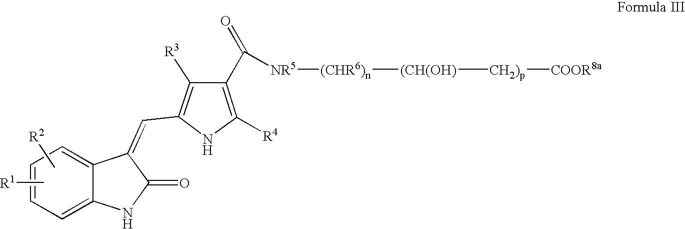
wherein R^{8a} is selected from the group consisting of hydrogen, (C1-C6) alkyl, and (C3-C8) cycloalkyl.

10. The compound, salt, tautomer, or prodrug according to claim 9, wherein:
R¹ and R² are independently selected from the group consisting of hydrogen and fluoro;
R³ and R⁴ are methyl;
R⁵, R⁶, and R^{8a} are hydrogen; and
n and p are independently 1, or 2.

11. The compound, salt, tautomer, or prodrug according to claim 10 selected from the group consisting of:

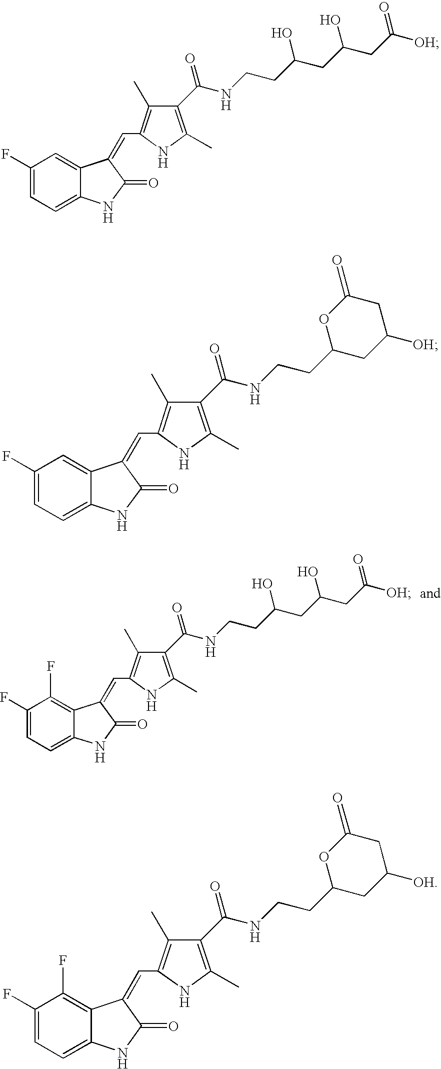

12. The compound, salt, tautomer, or prodrug according to claim 10 represented by the following structure:

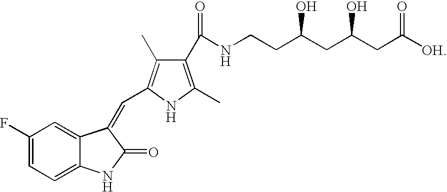

13. The compound, salt, tautomer, or prodrug according to claim 10 represented by the following structure:

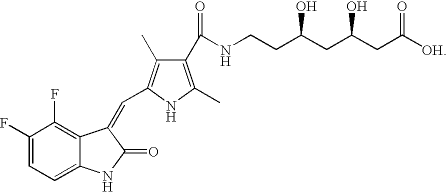

14. The compound, salt, tautomer, or prodrug according to claim 10 represented by the following structure:

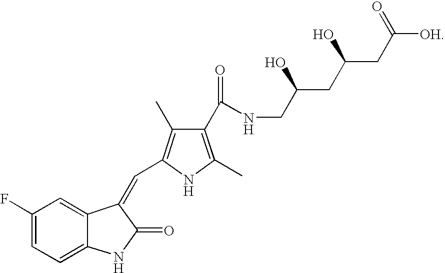

15. A compound, salt, tautomer, or prodrug according to claim 9 represented by Formula (IIIa): 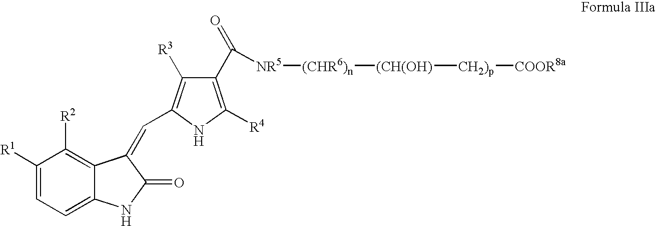
wherein:
R¹ and R² are independently selected from the group consisting of hydrogen and fluoro;
R³ and R⁴ are methyl;
R⁵, R⁶, and R^{8a} are hydrogen; and
n and p are 2.

16. A compound, salt, tautomer, or prodrug according to claim 15 represented by Formula (IIIb): 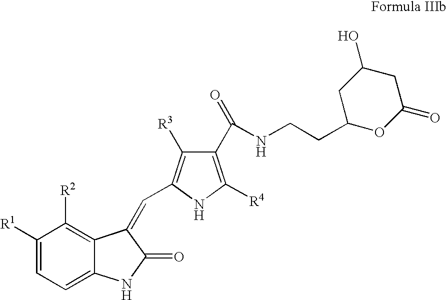
wherein:
R¹ and R² are independently selected from the group consisting of hydrogen and fluoro; and
R³ and R⁴ are methyl.

17. A compound, salt, tautomer, or prodrug according to claim 1 represented by Formula (IV): 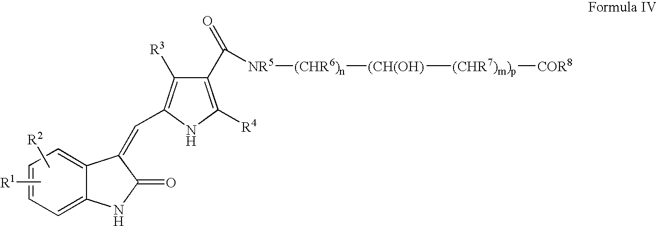
wherein R⁸ is NR⁹R¹⁰.

18. The compound, salt, tautomer, or prodrug of claim 17, wherein:
R¹ and R² are independently selected from the group consisting of hydrogen, halo, cyano;
R³, R⁴, R⁵ and R⁶ are independently hydrogen or (C1-C6))alkyl;
R⁷ is hydrogen, or hydroxyl;
n, and p are independently 1, or 2;
m is 0 or 1; and
R⁹ and R¹⁰ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphoric acid, (C1-C6) alkyl sulfuric acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁹ and R¹⁰ together with N forms a (C5-C8) heterocyclic ring either unsubstituted or substituted with one or more hydroxyls, ketones, ethers, and carboxylic acids.

19. The compound, salt, tautomer, or prodrug according to claim 18 selected from the group represented by the following structures:

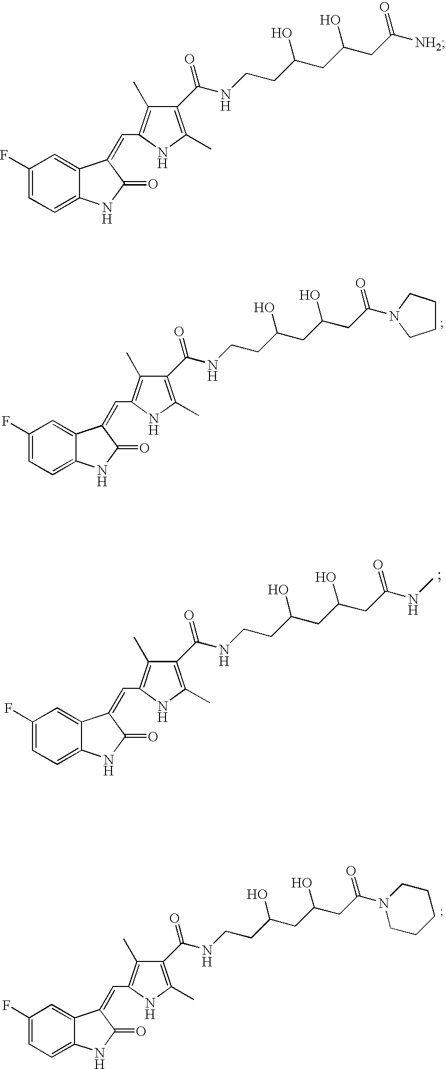

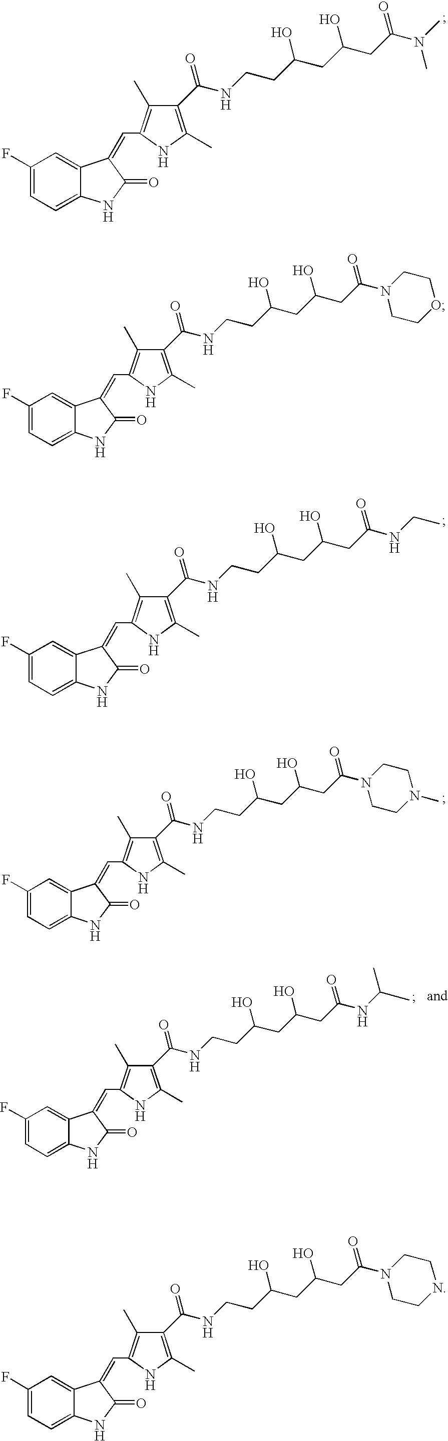

20. The compound, salt, tautomer, or prodrug according to claim 18 selected from the group represented by the following structures:

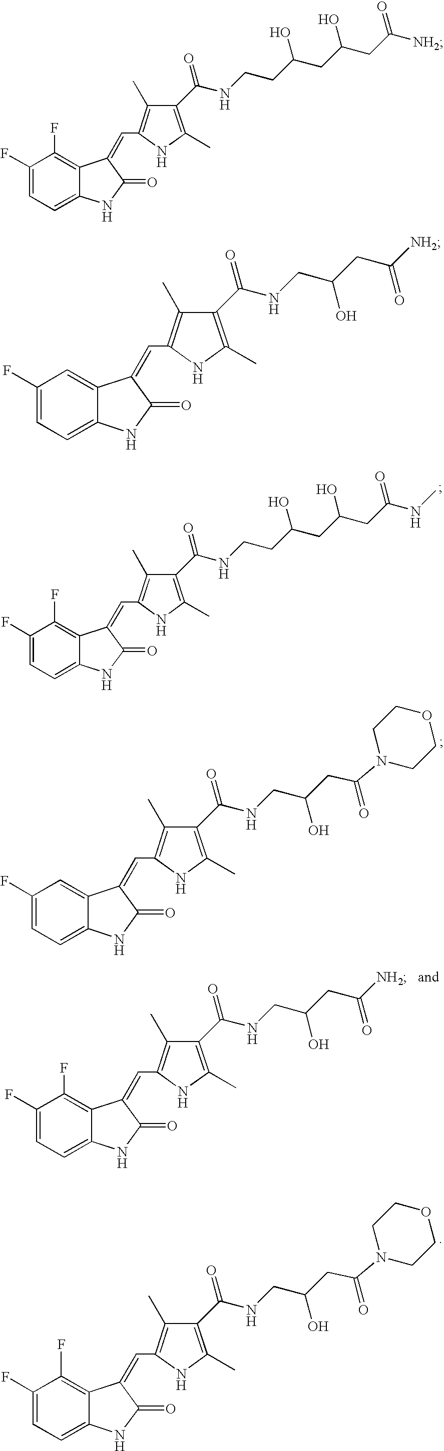

21. The compound, salt, tautomer, or prodrug according to claim 18 represented by the following structure:

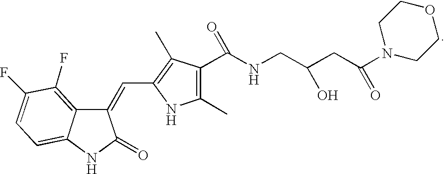

22. The compound, salt, tautomer, or prodrug according to claim 18 represented by the following structure:

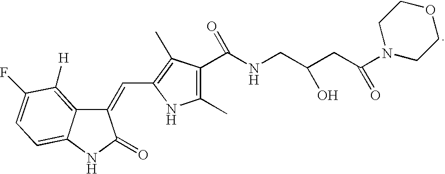

23. The compound, salt, tautomer, or prodrug according to claim 18 represented by the following structure:

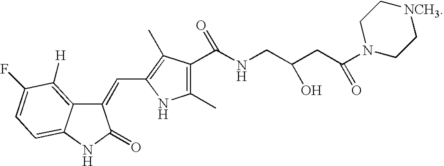

24. The compound, salt, tautomer, or prodrug according to claim 18 represented by the following structure: 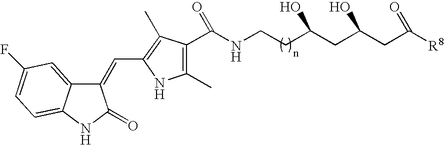
wherein n is 0, 1, or 2.

25. The compound, salt, tautomer, or prodrug according to claim 24 selected from the group represented by the following structures:

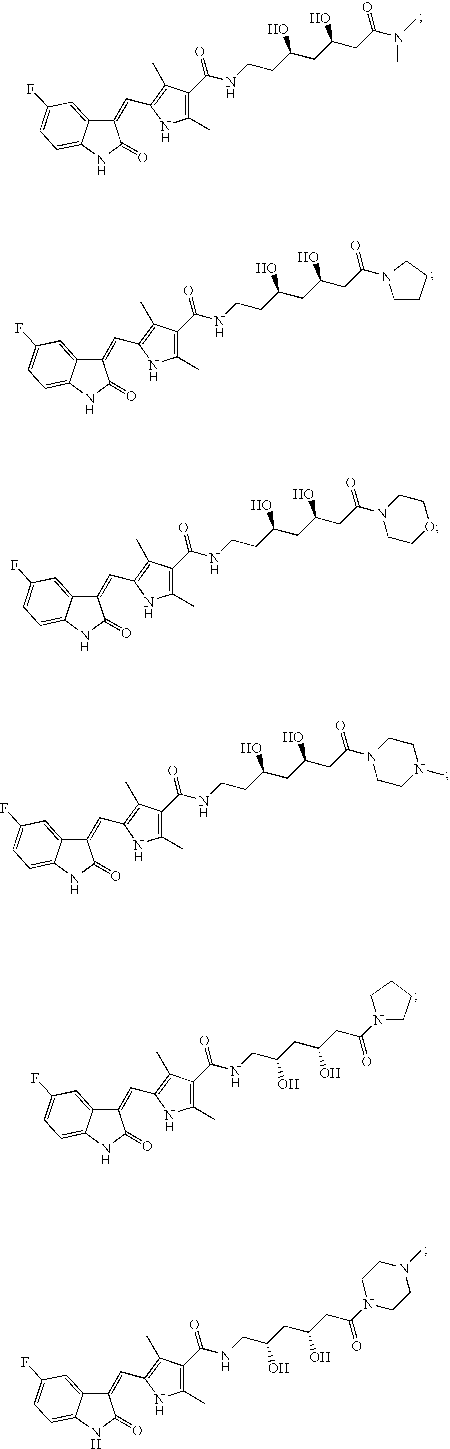

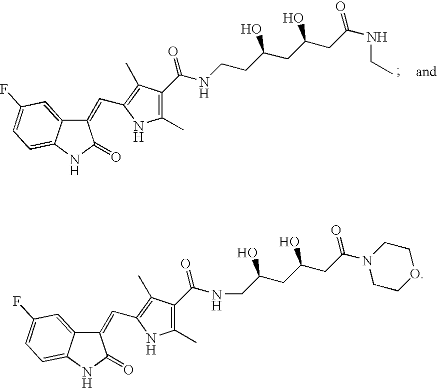

26. The compound, salt, tautomer, or prodrug according to claim 24 selected from the group represented by the following structures:

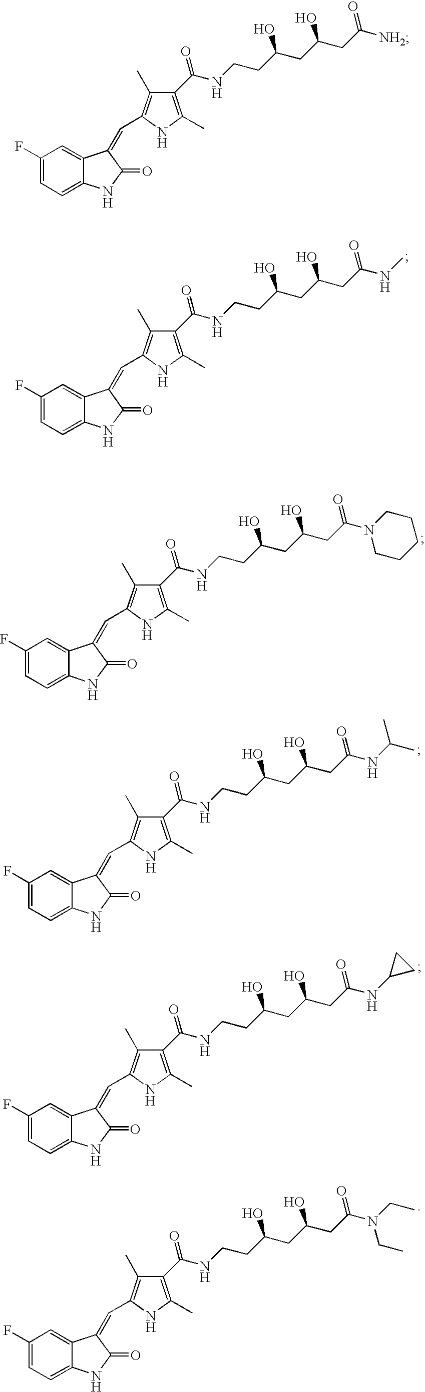

27. The compound, salt, tautomer, or prodrug according to claim 18 selected from the group represented by the following structures:

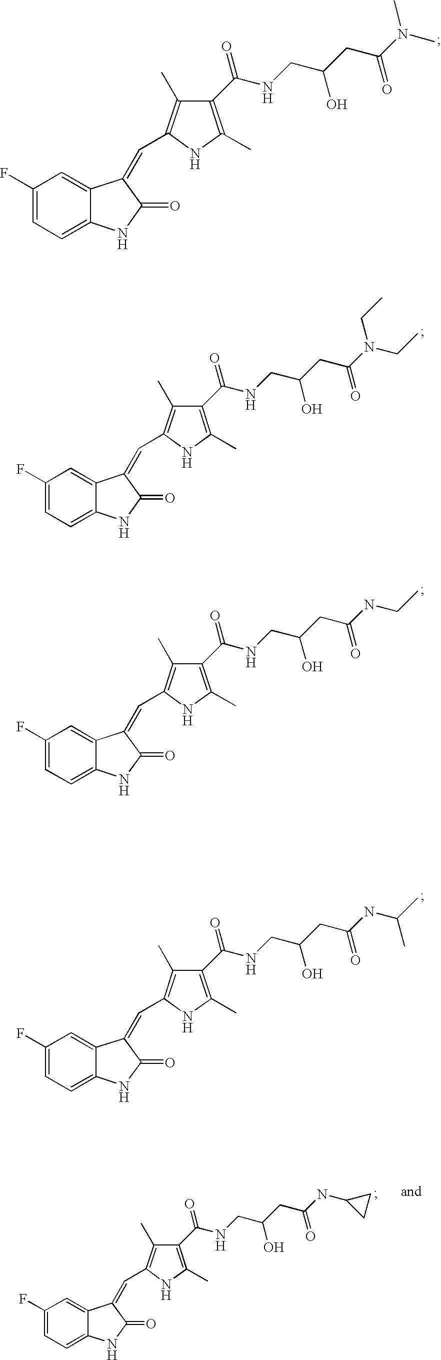

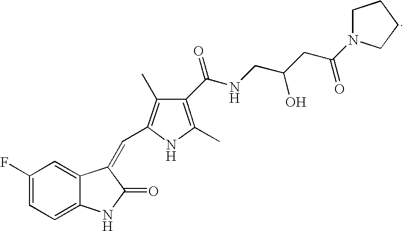

28. The compound, salt, tautomer, or prodrug according to claim 18 selected from the group represented by the following structures:

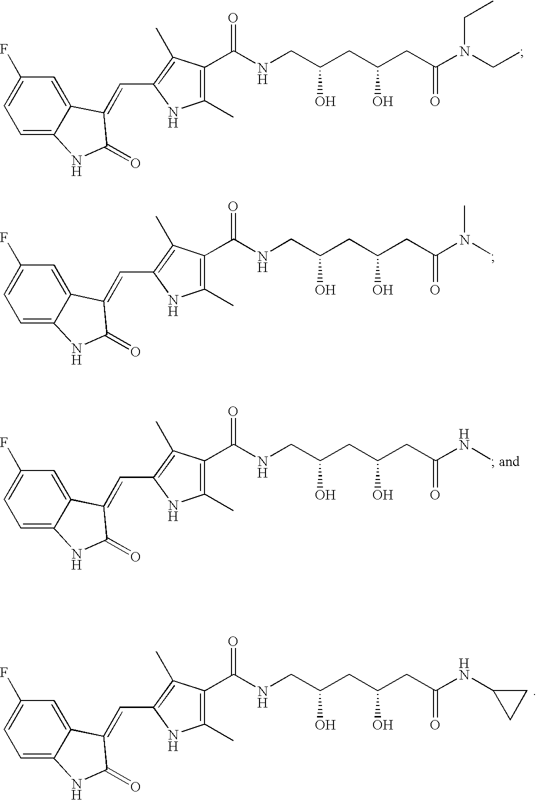

29. The compound, salt, tautomer, or prodrug according to claim 18 selected from the group represented by the following structures: 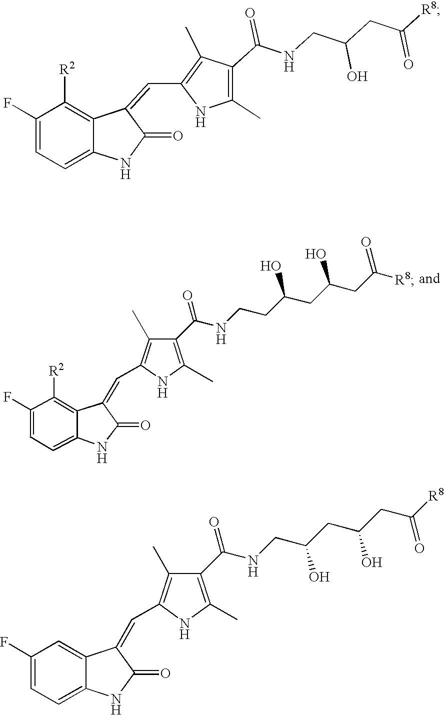
wherein:
R² is selected from the group consisting of hydrogen and fluoro; and
R⁸ is selected from the group consisting of radicals represented by the following structures:

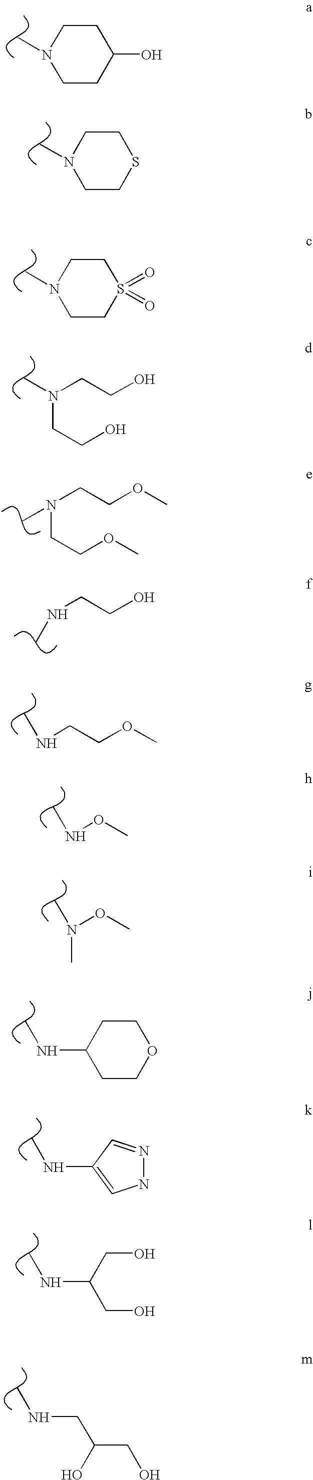

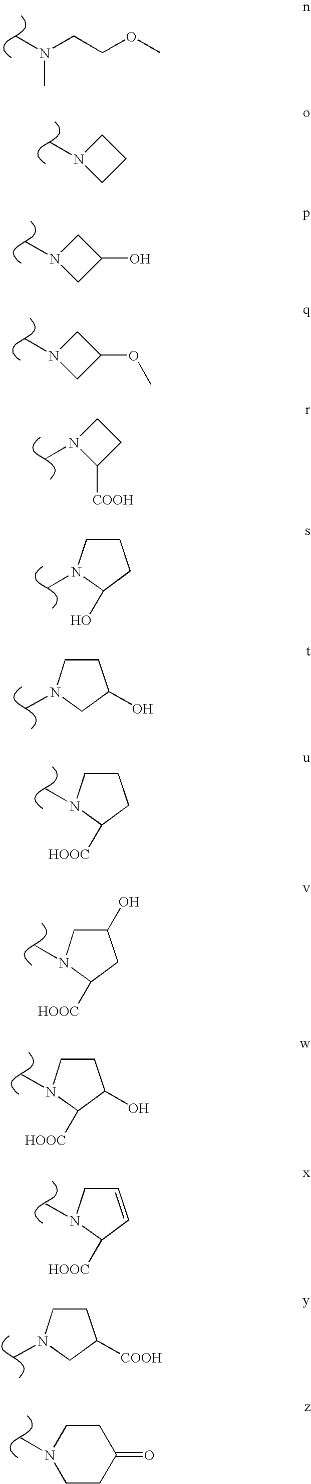

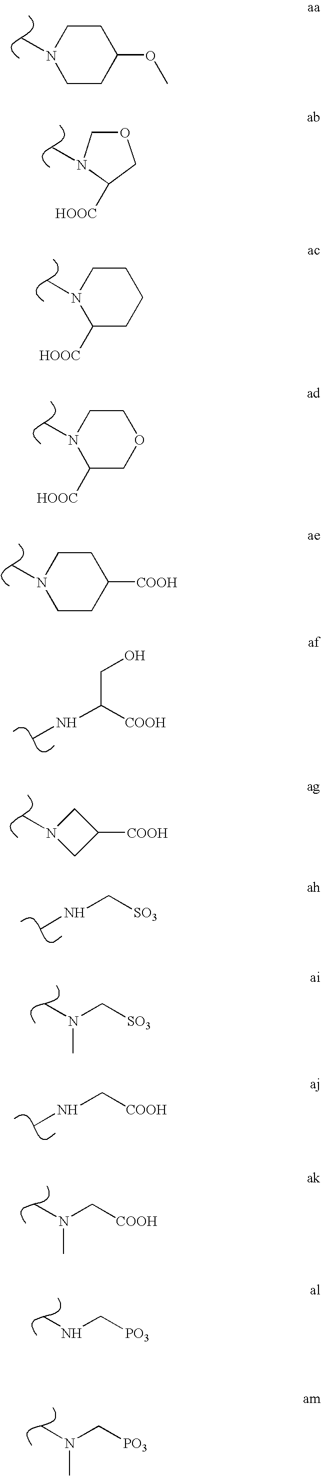

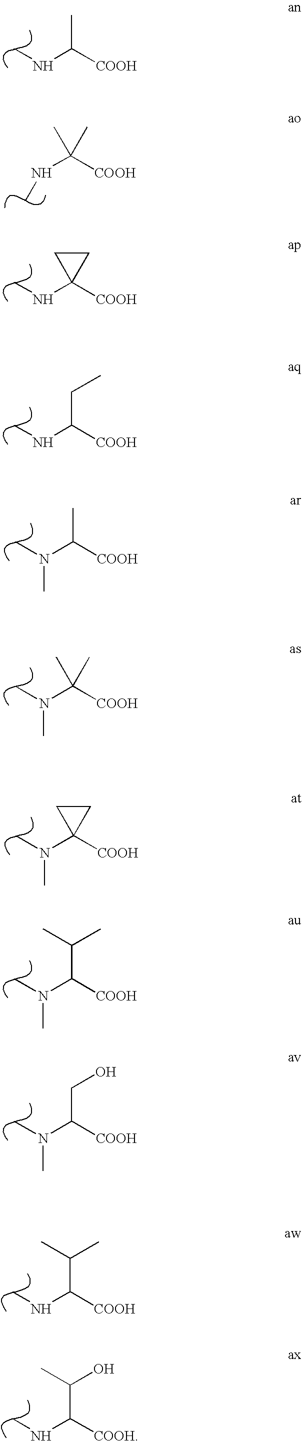

30. A compound according to claim 1 represented by Formula (V): 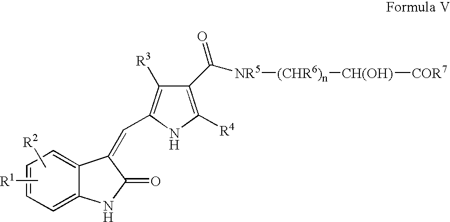
wherein:
R¹ is selected from the group consisting of hydrogen, halo, (C1-C6) alkyl, (C3-C8) cycloalkyl, (C1-C6) haloalkyl, hydroxy, (C1-C6) alkoxy, amino, (C1-C6) alkylamino, amide, sulfonamide, cyano, substituted or unsubstituted (C6-C10) aryl;
R² is selected from the group consisting of hydrogen, halo, (C1-C6) alkyl, (C3-C8) cycloalkyl, (C1-C6) haloalkyl, hydroxy, (C1-C6) alkoxy, (C2-C8) alkoxyalkyl, amino, (C1-C6) alkylamino, (C6-C10) arylamino;
R³ is selected from the group consisting of hydrogen, (C1-C6) alkyl, (C6-C10) aryl, (C5-C10) heteroaryl, and amide;
R⁴, R⁵ and R⁶ are independently selected from the group consisting of hydrogen and (C1-C6) alkyl;
R⁷ is selected from the group consisting of hydroxy, (C1-C6) O-alkyl, (C3-C8) O-cycloalkyl, and NR⁸R⁹; where R⁸ and R⁹ are independently selected from the group consisting of hydrogen, (C1-C6) alkyl, (C1-C6) hydroalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids; and
n is 1, 2, or 3;
or, a pharmaceutically acceptable salt, its tautomer, a pharmaceutically acceptable salt of its tautomer, or a prodrug thereof.

31. The compound, salt, tautomer, or prodrug according to claim 30 selected from the group represented by the following structures: 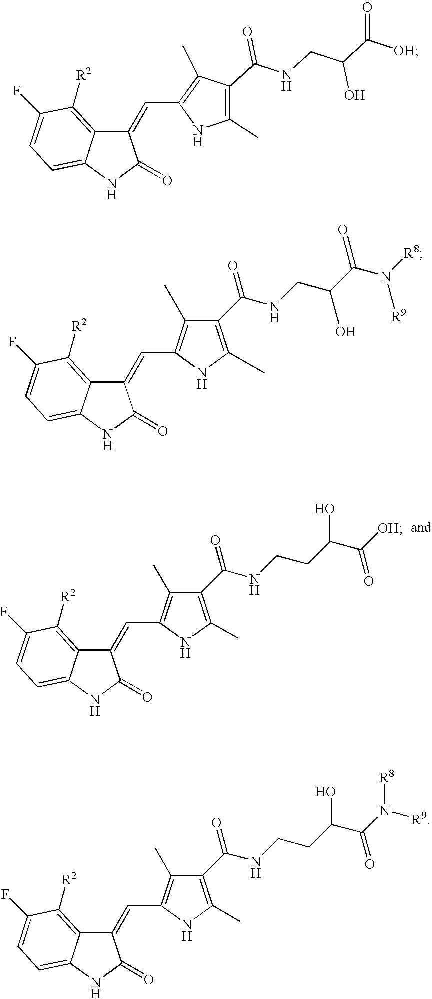
wherein R² is selected from the group consisting of hydrogen and fluoro.

32. The compound, salt, tautomer, or prodrug according to claim 30 represented by the following structure:

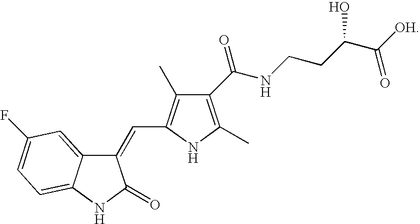

33. The compound, salt, tautomer, or prodrug according to claim 30 represented by Formula (VI): 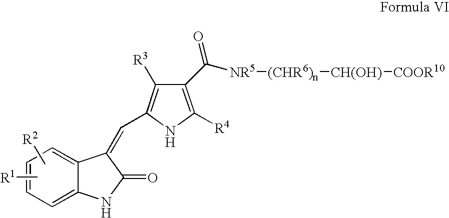
wherein R¹⁰ is selected from the group consisting of hydrogen, (C1-C6) alkyl, and (C3-C8) cycloalkyl.

34. The compound, salt, tautomer, or prodrug according to claim 33, wherein:
R¹ and R² are independently selected from the group consisting of hydrogen and fluoro;
R³ and R⁴ are methyl;
R⁵, R⁶, and R¹⁰ are hydrogen; and n is 1 or 2.

35. The compound, salt, tautomer, or prodrug according to claim 34 selected from the group consisting of:

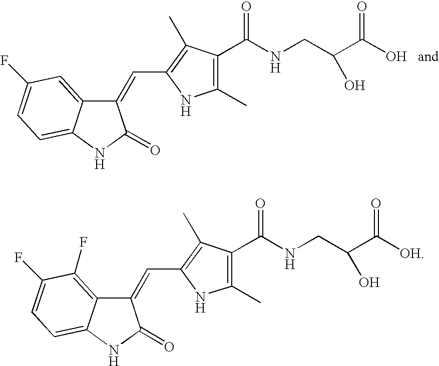

36. The compound, salt, tautomer, or prodrug according to claim 34 represented by the following structure:

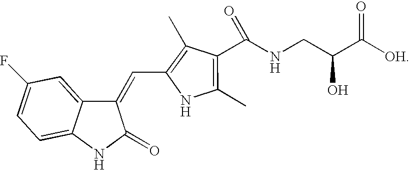

37. The compound, salt, tautomer, or prodrug represented by the following structure:

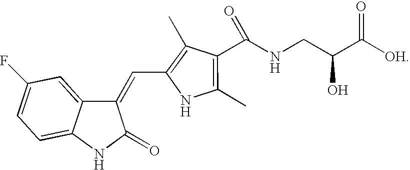

38. The compound, salt, tautomer, or prodrug according to claim 34 represented by the following structure:

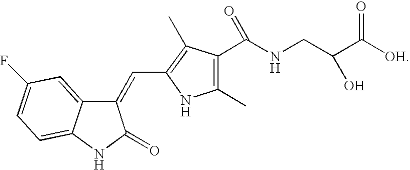

39. The compound, salt, tautomer, or prodrug according to claim 34 represented by the following structure:

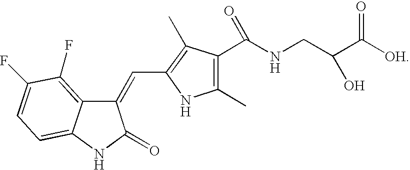

40. A compound, salt, tautomer, or prodrug according to claim 30 represented by Formula (VII):

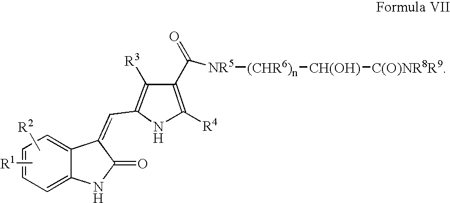

41. The compound, salt, tautomer, or prodrug of claim 38, wherein:
R¹ and R² are independently selected from the group consisting of hydrogen, halo, cyano;
R³, R⁴, R⁵ and R⁶ are independently hydrogen or (C1-C6))alkyl;
n is 1 or 2; and
R⁸ and R⁹ are selected from the group consisting of hydrogen, (C1-C6) alkyl, (C1-C6) hydroxyalkyl, (C1-C6) dihydroxyalkyl, (C1-C6) alkoxy, (C1-C6) alkyl carboxylic acid, (C1-C6) alkyl phosphonic acid, (C1-C6) alkyl sulfonic acid, (C1-C6) hydroxyalkyl carboxylic acid, (C1-C6) alkyl amide, (C3-C8) cycloalkyl, (C5-C8) heterocycloalkyl, (C6-C8) aryl, (C5-C8) heteroaryl, (C3-C8) cycloalkyl carboxylic acid, or R⁸ and R⁹ together with N forms a (C5-C8) heterocyclic ring either unsubstituted or substituted with one or more hydroxyls, ketones, ethers, and carboxylic acids.

42. The compound, salt, tautomer, or prodrug according to claim 41 selected from the group represented by the following structures:

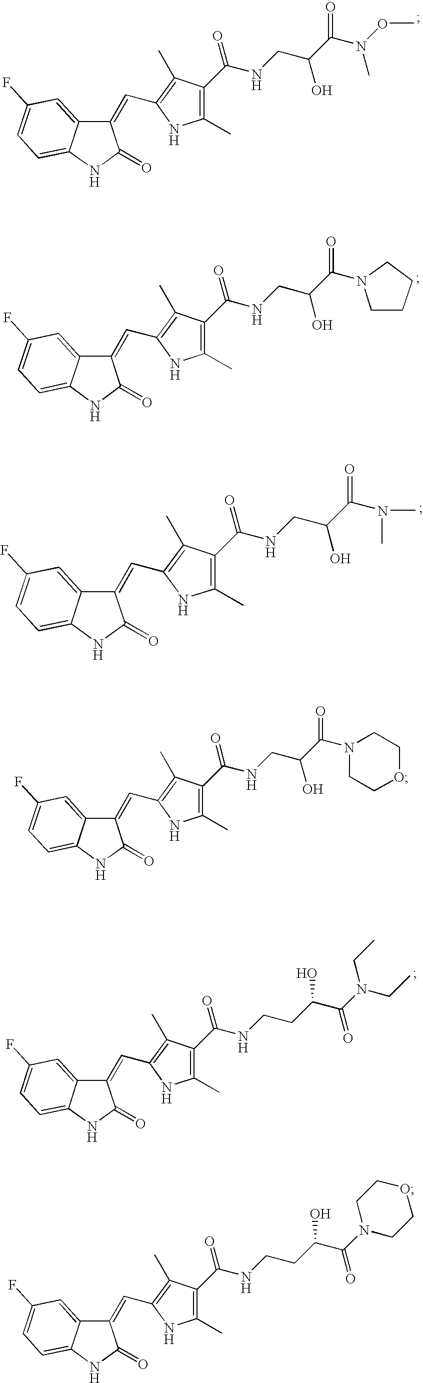

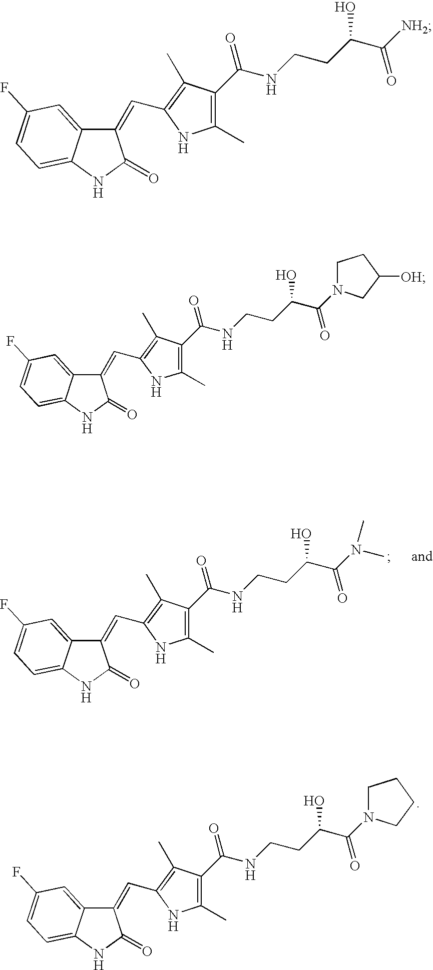

43. The compound, salt, tautomer, or prodrug according to claim 41 wherein n is 1.

44. The compound, salt, tautomer, or prodrug according to claim 41 represented by the following structures:

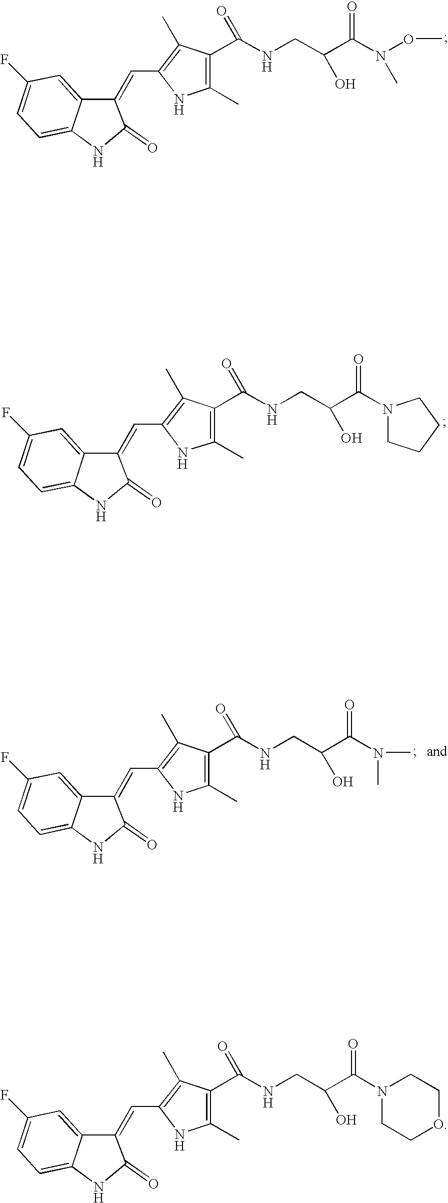

45. The compound, salt, tautomer, or prodrug according to claim 43 selected from the group represented by the following structures:

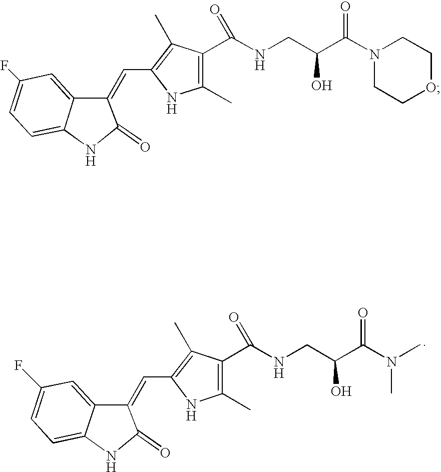

46. The compound, salt, tautomer, or prodrug according to claim 43 selected from the group represented by the following structures:

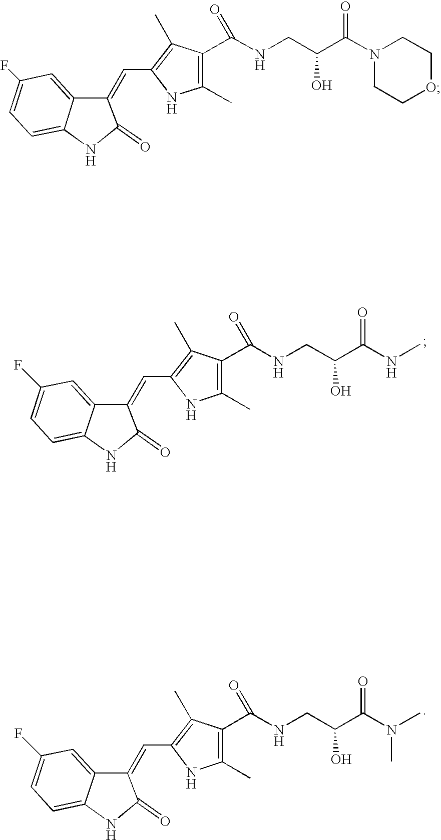

47. The compound, salt, tautomer, or prodrug represented by the following structure:

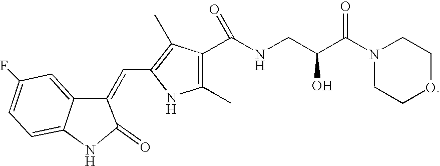

48. The compound, salt, tautomer, or prodrug represented by the following structure:

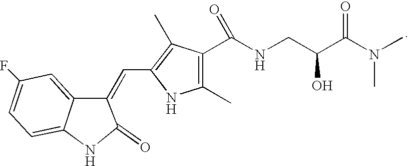

49. The compound, salt, tautomer, or prodrug represented by the following structure:

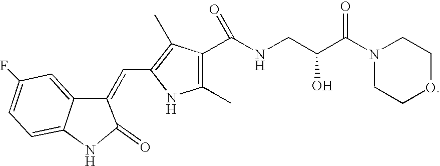

50. The compound, salt, tautomer, or prodrug according to claim 43 selected from the group represented by the following structures:

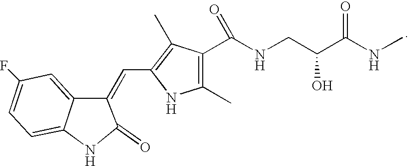

51. The compound, salt, tautomer, or prodrug according to claim 43 selected from the group represented by the following structures:

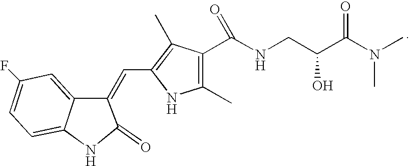

52. The compound, salt, tautomer, or prodrug according to claim 40 wherein n is 2.

53. The compound, salt, tautomer, or prodrug according to claim 52 represented by the following structures:

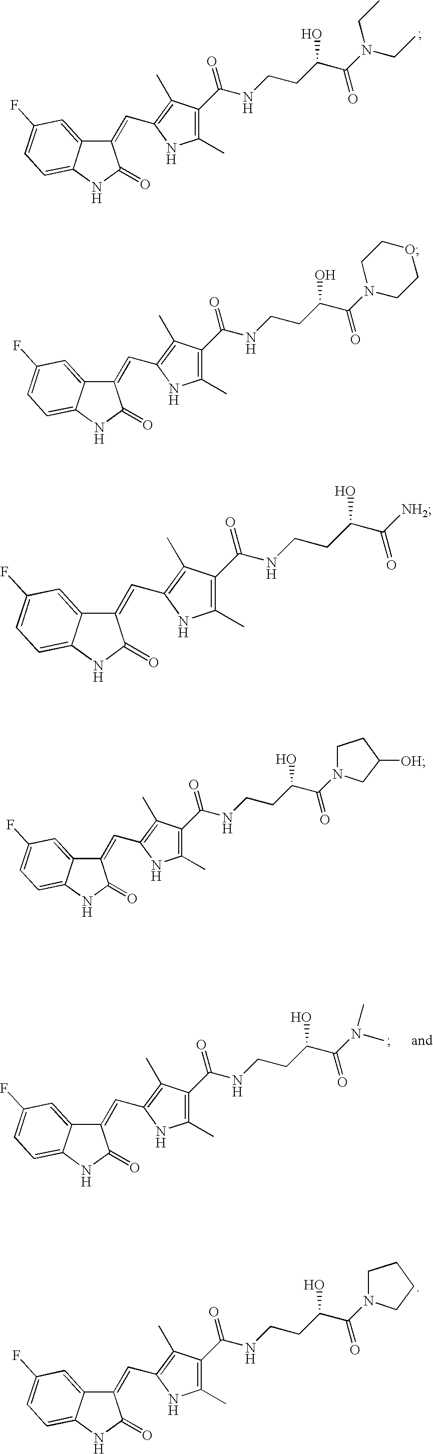

54. The compound, salt, tautomer, or prodrug according to claim 52 represented by the following structure:

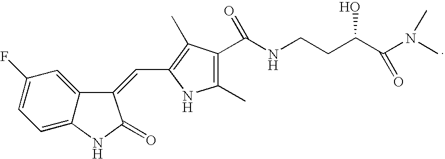

55. The compound, salt, tautomer, or prodrug according to claim 52 represented by the following structure:

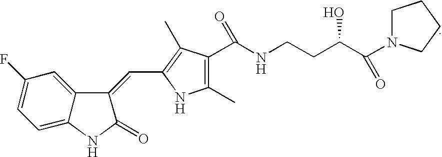

56. The compound, salt, tautomer, or prodrug according to claim 52 represented by the following structure:

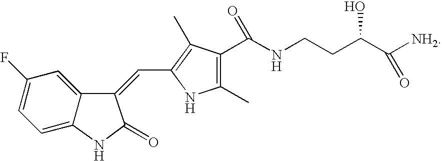

57. The compound, salt, tautomer, or prodrug according to claim 52 represented by the following structure:

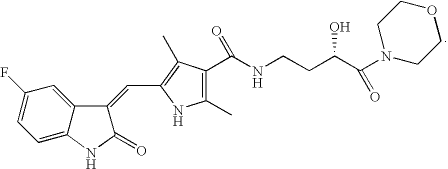

58. The compound, salt, tautomer, or prodrug according to claim 30 selected from the group represented by the following structures: 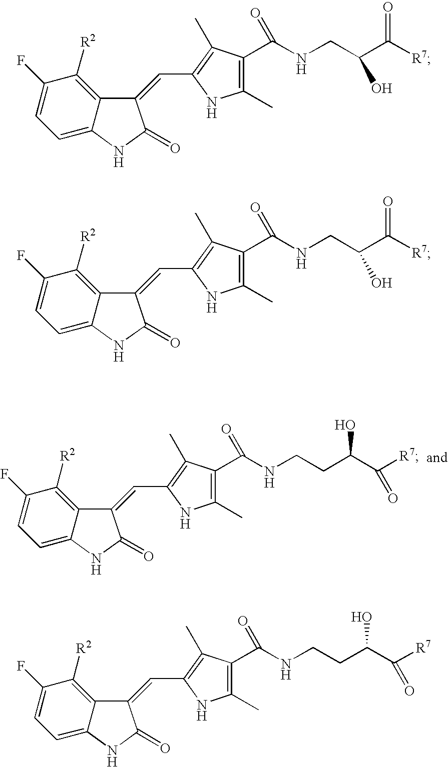
wherein:
R² is selected from the group consisting of hydrogen and fluoro; and
R⁷ is selected from the group consisting of hydroxyl or radicals represented by the following structures:

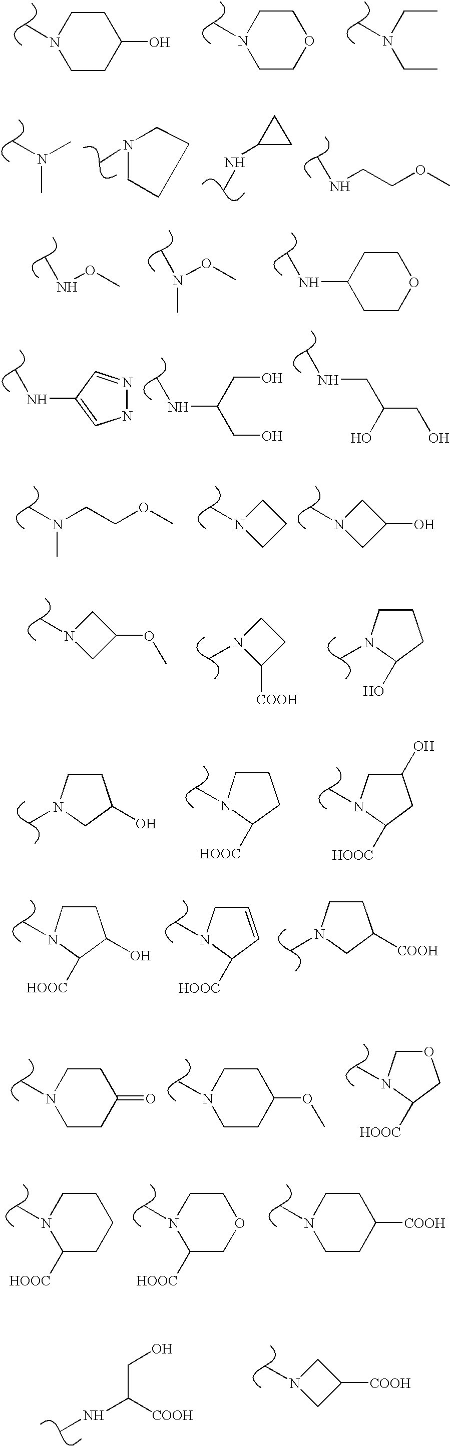

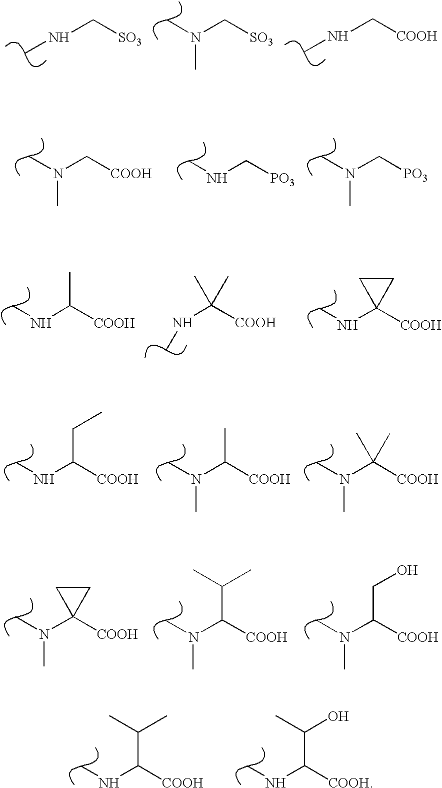

59. A method for the modulation of the catalytic activity of a protein kinase with a compound or salt of any one of claims 1-58.

60. The method of claim 59, wherein said protein kinase is selected from the group of receptors consisting of VEGF, PDGF, c-kit, Flt-3, Axl, and TrkA.

61. The method of claim 60, wherein said protein kinase is selected from the group of receptors consisting of VEGF and PDGF.

62. A method for the modulation of the catalytic activity of a protein kinase with a compound or salt of any one of claims 1-58.

63. The method of claim 61, wherein said protein kinase is selected from the group consisting of VEGF receptors and PDGF receptors.

64. The compound, salt, tautomer, or prodrug according to any of claims 1-58 with the following provisos:
the compound, salt, tautomer, or prodrug of claim 2 is excluded or
the compound, salt, tautomer, or prodrug of claim 3 is excluded or
the compound, salt, tautomer, or prodrug of claim 4 is excluded or
the compound, salt, tautomer, or prodrug of claim 5 is excluded or
the compound, salt, tautomer, or prodrug of claim 6 is excluded or
the compound, salt, tautomer, or prodrug of claim 7 is excluded or
the compound, salt, tautomer, or prodrug of claim 8 is excluded or
the compound, salt, tautomer, or prodrug of claim 9 is excluded or
the compound, salt, tautomer, or prodrug of claim 10 is excluded or
the compound, salt, tautomer, or prodrug of claim 11 is excluded or
the compound, salt, tautomer, or prodrug of claim 12 is excluded or
the compound, salt, tautomer, or prodrug of claim 13 is excluded or
the compound, salt, tautomer, or prodrug of claim 14 is excluded or
the compound, salt, tautomer, or prodrug of claim 15 is excluded or
the compound, salt, tautomer, or prodrug of claim 16 is excluded or
the compound, salt, tautomer, or prodrug of claim 17 is excluded or
the compound, salt, tautomer, or prodrug of claim 18 is excluded or
the compound, salt, tautomer, or prodrug of claim 19 is excluded or
the compound, salt, tautomer, or prodrug of claim 20 is excluded or
the compound, salt, tautomer, or prodrug of claim 21 is excluded or
the compound, salt, tautomer, or prodrug of claim 22 is excluded or
the compound, salt, tautomer, or prodrug of claim 23 is excluded or
the compound, salt, tautomer, or prodrug of claim 24 is excluded or
the compound, salt, tautomer, or prodrug of claim 25 is excluded or
the compound, salt, tautomer, or prodrug of claim 26 is excluded or
the compound, salt, tautomer, or prodrug of claim 27 is excluded or
the compound, salt, tautomer, or prodrug of claim 28 is excluded or
the compound, salt, tautomer, or prodrug of claim 29 is excluded or
the compound, salt, tautomer, or prodrug of claim 30 is excluded or
the compound, salt, tautomer, or prodrug of claim 31 is excluded or
the compound, salt, tautomer, or prodrug of claim 32 is excluded or
the compound, salt, tautomer, or prodrug of claim 33 is excluded or
the compound, salt, tautomer, or prodrug of claim 34 is excluded or
the compound, salt, tautomer, or prodrug of claim 35 is excluded or
the compound, salt, tautomer, or prodrug of claim 36 is excluded or
the compound, salt, tautomer, or prodrug of claim 37 is excluded or
the compound, salt, tautomer, or prodrug of claim 38 is excluded or
the compound, salt, tautomer, or prodrug of claim 39 is excluded or
the compound, salt, tautomer, or prodrug of claim 40 is excluded or
the compound, salt, tautomer, or prodrug of claim 41 is excluded or
the compound, salt, tautomer, or prodrug of claim 42 is excluded or
the compound, salt, tautomer, or prodrug of claim 43 is excluded or
the compound, salt, tautomer, or prodrug of claim 44 is excluded or
the compound, salt, tautomer, or prodrug of claim 45 is excluded or
the compound, salt, tautomer, or prodrug of claim 46 is excluded or
the compound, salt, tautomer, or prodrug of claim 47 is excluded or
the compound, salt, tautomer, or prodrug of claim 48 is excluded or
the compound, salt, tautomer, or prodrug of claim 49 is excluded or
the compound, salt, tautomer, or prodrug of claim 50 is excluded or
the compound, salt, tautomer, or prodrug of claim 51 is excluded or
the compound, salt, tautomer, or prodrug of claim 52 is excluded or
the compound, salt, tautomer, or prodrug of claim 53 is excluded or
the compound, salt, tautomer, or prodrug of claim 54 is excluded or
the compound, salt, tautomer, or prodrug of claim 55 is excluded or
the compound, salt, tautomer, or prodrug of claim 56 is excluded or
the compound, salt, tautomer, or prodrug of claim 57 is excluded or
the compound, salt, tautomer, or prodrug of claim 58 is excluded.
